SEQUENCE LISTING (2 sequences)

SEQ ID NO: 1 - DNA, 20 bp, synthetic construct
  source 1..20: mol_type = other DNA
ccttttcagaatagaactat

SEQ ID NO: 2 - protein, 4 aa, synthetic construct
GSGS

(19) United States
(12) Patent Application Publication
(10) Pub. No.: US 2025/0368620 A1
(43) Pub. Date: Dec. 4, 2025

(54) ARNT Degrading Compounds and Uses Thereof

(71) Applicant: Celgene Corporation, Princeton, NJ (US)

(72) Inventors: Bryan J. Simmons, Princeton, NJ (US); Mimi Quan, Princeton, NJ (US); Jennifer Riggs, Princeton, NJ (US); Christoph W. Zapf, Princeton, NJ (US); Alexandre Hentz, Toulouse (FR); Christophe Muller, Toulouse (FR); Kim Spielmann, Toulouse (FR); Arnaud Piettre, Toulouse (FR); Sandra Muller, Toulouse (FR); Joshua M. Baughman, Princeton, NJ (US)

(73) Assignee: Celgene Corporation, Princeton, NJ (US)

(21) Appl. No.: 19/221,762

(22) Filed: May 29, 2025

Related U.S. Application Data
(60) Provisional application No. 63/653,427, filed on May 30, 2024.

(51) Int. Cl. C07D 401/14; A61K 31/454; A61K 31/4545; C07D 471/08; C07D 487/08

(52) U.S. Cl. CPC C07D 401/14; A61K 31/454; A61K 31/4545; C07D 471/08; C07D 487/08

(57) ABSTRACT

Disclosed herein are ARNT degrading compounds, and use of the compounds for treating diseases and conditions associated with the aryl hydrocarbon receptor nuclear translocator (ARNT) protein. Also disclosed herein are pharmaceutical compositions comprising such compounds, e.g., for use in the disclosed methods. In certain embodiments, the compounds are of the following structural formula:

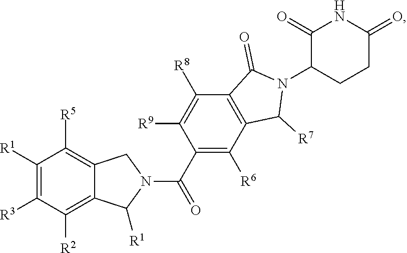

wherein values for the variables (e.g., R¹, R², R³, R⁴, R⁵, R⁶, R⁷, R⁸, R⁹) are as described herein.

### CROSS-REFERENCE TO RELATED APPLICATION

This application claims the benefit of U.S. Provisional Application No. 63/653,427, filed May 30, 2024, which is incorporated herein by reference in its entirety for any purpose.

### SEQUENCE LISTING

The present application contains a Sequence Listing which has been submitted electronically in XML format. Said XML copy, created on May 22, 2025, is named “01277-0034-00PCT.xml” and is 2,785 bytes in size. The information in the electronic format of the sequence listing is incorporated herein by reference in its entirety.

### FIELD

The present disclosure relates generally to compounds, compositions, and methods for their preparation, and use of the compounds and compositions for treating diseases and conditions associated with the aryl hydrocarbon receptor nuclear translocator (ARNT) protein.

### BACKGROUND

Aryl hydrocarbon receptor nuclear translocator (ARNT), also known as hypoxia inducible factor 1 beta (HIF-1β), is a transcription factor which can heterodimerize with other basic helix-loop-helix proteins. For example, ARNT has been shown to heterodimerize with hypoxia inducible factor 1 alpha (HIF-1α) and hypoxia inducible factor 2 alpha (HIF-2α. Both HIF-1α and HIF-2α form a dimeric complex with ARNT and subsequently bind to hypoxia response elements (HRE) in target genes.

ARNT serves as a key mediator for adaptive hypoxia and xenobiotic responses. Certain cancers carrying von Hippel-Lindau (VHL) mutations are characterized by hypoxia inducible factor stabilization and hyperactive hypoxia inducible factor transcription. Hypoxia is a driving force in cancer progression and is closely linked to poor patient prognosis and resistance to chemotherapy and radiation treatment.

Therefore, the identification of agents that block or disrupt the hypoxic response pathway in cancers, such as small molecules that modulate the activity of ARNT, is desirable.

### SUMMARY

Provided herein are compounds of structural formula (I):

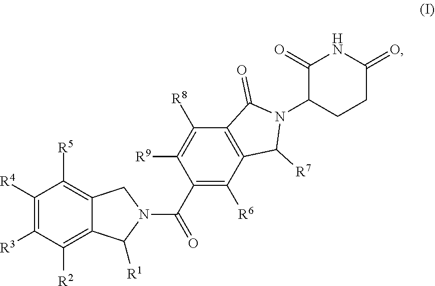

and pharmaceutically acceptable salts, tautomers, isotopologues, and/or stereoisomers thereof thereof, wherein values for the variables (e.g., R¹, R², R³, R⁴, R⁵, R⁶, R⁷, R⁸, R⁹) are as described herein.

Also provided herein are pharmaceutical compositions comprising a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof, and a pharmaceutically acceptable carrier, excipient or vehicle.

Also provided herein are methods of modulating ARNT activity in a subject in need thereof, comprising administering to the subject a therapeutically effective amount of a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof.

Also provided herein are methods of treating cancer in a subject in need thereof, comprising administering to the subject a therapeutically effective amount of a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof.

Also provided herein are uses of a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof, e.g., for modulating ARNT activity; treating cancer, such as renal cell carcinoma.

Also provided herein is a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof, or a pharmaceutical composition thereof, for use as a medicament, e.g., for modulating ARNT activity; treating cancer, such as renal cell carcinoma.

Also provided herein is a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof, for manufacture of a medicament, e.g., for a use described herein, such as modulating ARNT activity; treating cancer, such as renal cell carcinoma.

Also provided herein is a compound of the present disclosure, e.g., a compound of structural formula (I), or a substructure thereof, such as a compound of structural formula (Ia), (Ib), (Ic), or (Id), or of Table 1, or a pharmaceutically acceptable salt, tautomer, isotopologue, or stereoisomer thereof, or a pharmaceutical composition thereof, for use as described herein (e.g., modulating ARNT activity; treating cancer, such as renal cell carcinoma).

The present embodiments can be understood more fully by reference to the detailed description and examples, which are intended to exemplify non-limiting embodiments.

### DETAILED DESCRIPTION

### Definitions

As used herein, the terms “comprising” and “including” can be used interchangeably. The terms “comprising” and “including” are to be interpreted as specifying the presence of the stated features or components as referred to, but do not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

The term “consisting of” means that a subject-matter has at least 90%, 95%, 97%, 98% or 99% of the stated features or components of which it consists. In another embodiment, the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein and unless otherwise specified, an “alkyl” group is a saturated, straight chain or branched, non-cyclic hydrocarbon having from 1 to 10 carbon atoms, typically from 1 to 8 carbons or, in some embodiments, from 1 to 6, 1 to 4, 1 to 3, or 2 to 6 carbon atoms. Representative straight chain alkyl groups include -methyl, -ethyl, -n-propyl, -n-butyl, -n-pentyl and -n-hexyl; while branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like.

Alkyl groups can be substituted or unsubstituted. When the alkyl groups described herein are said to be “substituted,” they may be substituted with any substituent or substituents as those found in the exemplified compounds and embodiments disclosed herein, as well as halogen; hydroxy; alkoxy; cycloalkyloxy, aryloxy, heterocyclyloxy, heteroaryloxy, heterocycloalkyoxy, cycloalkylalkyloxy, aralkyloxy, heterocyclylalkyloxy, heteroarylalkyloxy, heterocycloalkyalkyloxy; oxo (═O); amino, alkylamino, cycloalkylamino, arylamino, heterocyclylamino, heteroarylamino, heterocycloalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In some embodiments, one or more hydrogens, such as one, two, three, four, or five hydrogens, in an alkyl, alkenyl, or alkynyl group may be replaced with halogen.

As used herein and unless otherwise specified, a “cycloalkyl” group is a saturated, or partially saturated cyclic hydrocarbon having from 3 to 10 carbon atoms in a single ring or multiple condensed, spiro, or bridged rings, which can be optionally substituted. In some embodiments, a cycloalkyl group has 3 to 8 ring carbon atoms, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. In some embodiments, a cycloalkyl is saturated.

As used herein and unless otherwise specified, an “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryl groups include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

As used herein and unless otherwise specified, “hetero” refers to an atom that is not carbon or hydrogen. Suitable heteroatoms include oxygen, sulfur, and nitrogen.

As used herein and unless otherwise specified, a “heteroaryl” group is an aromatic ring system having one to four heteroatoms as ring atoms in a heteroaromatic ring system, wherein the remainder of the ring atoms are carbon atoms. In some embodiments, heteroaryl groups contain 3 to 6 ring atoms, and in others from 5 to 9, 6 to 9, 5 to 10, or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indol-2-onyl), isoindolin-1-onyl, azaindolyl, pyrrolopyridyl (e.g., 1H-pyrrolo[2,3-b]pyridyl), indazolyl, benzimidazolyl (e.g., 1H-benzo[d]imidazolyl), azabenzimidazolyl, imidazopyridyl (e.g., 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl, 3,4-dihydroisoquinolin-1(2H)-onyl, tetrahydroquinolinyl, quinoxalinyl, and quinazolinyl groups. A heteroaryl group can be substituted or unsubstituted. The phrase “heteroaryl groups” also includes groups containing fused rings, such as fused heteroaromatic-aliphatic or fused heteroaromatic-heteroaliphatic ring systems.

As used herein and unless otherwise specified, a “heterocyclyl” or “heterocycloalkyl” is a cycloalkyl in which one to four of the ring carbon atoms are independently replaced with a heteroatom. Suitable heteroatoms include oxygen, sulfur and nitrogen. In some embodiments, heterocyclyl groups include 3 to 10 ring members, whereas other such groups have 3 to 5, 3 to 6, or 3 to 8 ring members. In some embodiments, a heterocyclyl group has 4 to 7 ring members. A heterocyclyl group can be substituted or unsubstituted. When a heterocyclyl is substituted, a substituent can be bonded to the heterocyclyl at any ring atom (i.e., at any carbon atom or heteroatom of the heterocyclic ring). The phrase includes fused, spiro, and bridged polycyclic ring systems containing a heteroatom such as, but not limited to, quinuclidyl. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. In some embodiments, heterocyclyl is saturated.

As used herein and unless otherwise specified, a “cycloalkylalkyl” group is a radical of the formula: -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopropylmethyl, cyclobutylmethyl, cyclopentylmethyl, cyclohexylmethyl, cyclopropylethyl, cyclobutylethyl, cyclopentylethyl, cyclohexylethyl, cyclopentylpropyl, cyclohexylpropyl and the like.

As used herein and unless otherwise specified, an “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and aralkyl groups wherein the aryl group is fused to a cycloalkyl group such as indan-4-yl ethyl.

As used herein and unless otherwise specified, a “heterocyclylalkyl” group is a radical of the formula: -alkyl-heterocyclyl, wherein alkyl and heterocyclyl are defined above. A “heteroarylalkyl” group is a radical of the formula: -alkyl-heteroaryl, wherein alkyl and heteroaryl are defined above. Substituted heterocyclylalkyl groups may be substituted at the alkyl, the heterocyclyl, or both the alkyl and the heterocyclyl portions of the group. Representative heterocylylalkyl groups include but are not limited to morpholin-4-yl ethyl, morpholin-4-yl propyl, furan-2-yl methyl, furan-3-yl methyl, pyridin-3-yl methyl, tetrahydrofuran-2-yl ethyl, and indol-2-yl propyl.

As used herein and unless otherwise specified, “halogen” or “halo” is fluorine, chlorine, bromine, or iodine. In some embodiments, halogen is fluorine, chlorine, or bromine. In some embodiments, halogen is fluorine or chlorine. In some embodiments, halogen is fluorine or bromine. In some embodiments, halogen is chlorine or bromine.

As used herein and unless otherwise specified, a “haloalkyl” group is an alkyl group as described above substituted with one or more independently selected halogens, wherein halogen is as described above. In some embodiments, haloalkyl is perhalogenated. Examples of haloalkyl include but are not limited to difluoromethyl, trifluoromethyl, chlorofluoromethyl, and the like.

As used herein and unless otherwise specified, a “hydroxyalkyl” group is an alkyl group as described above substituted with one or more hydroxy groups.

As used herein and unless otherwise specified, an “alkoxy” group is —O-(alkyl), wherein alkyl is defined above. An “alkylthio” group is —S-(alkyl), wherein alkyl is defined above.

As used herein and unless otherwise specified, an “alkoxyalkyl” group is -(alkyl)-O-(alkyl), wherein alkyl is defined above.

As used herein and unless otherwise specified, a “cycloalkyloxy” group is —O-(cycloalkyl), wherein cycloalkyl is defined above.

As used herein and unless otherwise specified, an “aryloxy” group is —O-(aryl), wherein aryl is defined above.

As used herein and unless otherwise specified, a “heterocyclyloxy” group is —O-(heterocyclyl), wherein heterocyclyl is defined above. A “heteroaryloxy” group is —O-(heteroaryl), wherein heteroaryl is defined above.

As used herein and unless otherwise specified, an “amino” group is a radical of the formula: —NH₂, —NH(R^{#}), or —N(R^{#})₂, wherein each R^{#} is independently an alkyl, cycloalkyl, cycloalkylalkyl, aryl, aralkyl, heterocyclyl (e.g., heteroaryl or heterocycloalkyl), or heterocyclylalkyl (e.g., heteroarylalkyl or heterocycloalkylalkyl) group defined above, each of which is independently substituted or unsubstituted.

In one embodiment, an “amino” group is an “alkylamino” group, which is a radical of the formula: —NH-alkyl or —N(alkyl)₂, wherein each alkyl is independently defined above. The term “cycloalkylamino”, “arylamino”, “heterocyclylamino”, “heteroarylamino”, “heterocycloalkylamino”, or the like, mirrors the above description for “alkylamino” where the term “alkyl” is replaced with “cycloalkyl”, “aryl”, “heterocyclyl”, “heteroaryl”, “heterocycloalkyl”, or the like, respectively.

As used herein and unless otherwise specified, a “carboxy” group is a radical of the formula: —C(O)OH.

As used herein and unless otherwise specified, an “acyl” group is a radical of the formula: —C(O)(R^{#}) or —C(O)H, wherein R^{#} is defined above. A “formyl” group is a radical of the formula: —C(O)H.

As used herein and unless otherwise specified, an “amido” group is a radical of the formula: —C(O)—NH₂, —C(O)—NH(R^{#}), —C(O)—N(R^{#})₂, —NH—C(O)H, —NH—C(O)—(R^{#}), —N(R^{#})—C(O)H, or —N(R^{#})—C(O)—(R^{#}), wherein each R^{#} is independently defined above.

In one embodiment, an “amido” group is an “aminocarbonyl” group, which is a radical of the formula: —C(O)—NH₂, —C(O)—NH(R^{#}), —C(O)—N(R^{#})₂, wherein each R^{#} is independently defined above.

In one embodiment, an “amido” group is an “acylamino” group, which is a radical of the formula: —NH—C(O)H, —NH—C(O)—(R^{#}), —N(R^{#})—C(O)H, or —N(R)—C(O)—(R^{#}), wherein each R^{#} is independently defined above.

As used herein and unless otherwise specified, a “sulfonylamino” group is a radical of the formula: —NHSO₂(R^{#}) or —N(R^{#})SO₂(R^{#}), wherein each R^{#} is defined above.

As used herein and unless otherwise specified, an “ester” group is a radical of the formula: —C(O)—O—(R^{#}) or —O—C(O)—(R^{#}), wherein R^{#} is defined above.

In one embodiment, an “ester” group is an “alkoxycarbonyl” group, which is a radical of the formula: —C(O)—O-(alkyl), wherein alkyl is defined above. The term “cycloalkyloxycarbonyl”, “aryloxycarbonyl”, “heterocyclyloxycarbonyl”, “heteroaryloxycarbonyl”, “heterocycloalkyloxycarbonyl”, or the like, mirrors the above description for “alkoxycarbonyl” where the term “alkoxy” is replaced with “cycloalkyloxy”, “aryloxy”, “heterocyclyloxy”, “heteroaryloxy”, “heterocycloalkyloxy”, or the like, respectively.

As used herein and unless otherwise specified, a “carbamate” group is a radical of the formula: —O—C(O)—NH₂, —O—C(O)—NH(R^{#}), —O—C(O)—N(R^{#})₂, —NH—C(O)—O—(R^{#}), or —N(R^{#})—C(O)—O—(R^{#}), wherein each R^{#} is independently defined above.

As used herein and unless otherwise specified, a “urea” group is a radical of the formula: —NH(CO)NH₂, —NHC(O)NH(R^{#}), —NHC(O)N(R^{#})₂, —N(R^{#})C(O)NH₂, —N(R^{#})C(O)NH(R^{#}), or —N(R^{#})C(O)N(R^{#})₂, wherein each R^{#} is independently defined above.

As used herein and unless otherwise specified, a “sulfinyl” group is a radical of the formula: —S(O)R^{#}, wherein R^{#} is defined above.

As used herein and unless otherwise specified, a “sulfonyl” group is a radical of the formula: —S(O)₂R^{#}, wherein R^{#} is defined above.

As used herein and unless otherwise specified, an “aminosulfonyl” group is a radical of the formula: —SO₂NH₂, —SO₂NH(R^{#}), or —SO₂N(R^{#})₂, wherein each R^{#} is independently defined above.

When the groups described herein, with the exception of alkyl groups, are said to be “substituted,” they may be substituted with any appropriate substituent or substituents. Illustrative examples of substituents are those found in the exemplary compounds and embodiments disclosed herein, as well as halogen; alkyl, alkenyl, alkynyl, cycloalkyl, aryl, heterocyclyl, heteroaryl, cycloalkylalkyl, aralkyl, heterocyclylalkyl, heteroarylalkyl, optionally further substituted; hydroxy; alkoxy; cycloalkyloxy, aryloxy, heterocyclyloxy, heteroaryloxy, cycloalkylalkyloxy, aralkyloxy, heterocyclylalkyloxy, heteroarylalkyloxy; oxo (═O); oxide (e.g., a nitrogen atom substituted with an oxide is called N-oxide); amino, alkylamino, cycloalkylamino, arylamino, heterocyclylamino, heteroarylamino, imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In some embodiments, one or more hydrogens, such as one, two, three, four, or five hydrogens, in a substituent may be replaced with halogen. In some embodiments, substitution replaces a hydrogen atom with alkyl, alkoxy, aryloxy, halogen, or haloalkyl.

As used herein, the term “pharmaceutically acceptable salt(s)” refers to a salt prepared from a pharmaceutically acceptable non-toxic acid or base including inorganic acids and bases and organic acids and bases. Suitable pharmaceutically acceptable base addition salts include, but are not limited to metallic salts made from aluminum, calcium, lithium, magnesium, potassium, sodium and zinc, or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Other salts are well-known in the art, see for example, Remington's PharmaceuticalSciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. Compounds can have chiral centers and can occur as racemates, individual enantiomers or diastereomers, and mixtures thereof. Unless otherwise specified, all such isomeric forms are included within the embodiments disclosed herein, including mixtures thereof.

The use of stereomerically pure forms of compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that compounds can include E and Z isomers, or cis and trans isomers. Unless otherwise specified, all such isomeric forms are included within the embodiments disclosed herein, including mixtures thereof.

“Tautomers” refers to isomeric forms of a compound that are in equilibrium with each other through the migration of a proton. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other.

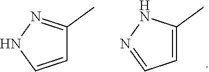

As readily understood by one skilled in the art, a wide variety of functional groups and other structures may exhibit tautomerism. Unless otherwise specified, all tautomeric forms of the compounds of the present disclosure are within the scope of the present disclosure.

It should also be noted that compounds can contain unnatural proportions of atomic isotopes of at least one of the atoms. For example, compounds may be radiolabeled with radioactive isotopes, for example, tritium (3H), iodine-125 (¹²⁵I), sulfur-35 (³⁵S), or carbon-14 (¹⁴C), or may be isotopically enriched, such as with carbon-13 (¹³C) or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. Unless otherwise specified, all isotopic variations of compounds of the present disclosure, whether radioactive or not, are intended to be encompassed within the scope of the embodiments provided herein. In some embodiments, there are provided isotopologues of the compounds, for example, the isotopologues are carbon-13, or nitrogen-15 enriched. As used herein, “deuterated”, means a compound wherein at least one hydrogen (H) has been replaced by deuterium (indicated by D or ²H), that is, the compound is enriched in deuterium in at least one position. In some embodiments, an isotopologue is a deuterated isotopologue of a specified compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

The term “therapeutically effective amount” means an amount capable of treating a disorder, disease or condition, or symptoms thereof.

The term “subject” or “patient” includes humans.

Unless otherwise defined, the technical and scientific terms used herein have the same meaning as commonly understood by one of ordinary skill in the art to which this application belongs.

### Compounds

Provided herein is a compound of structural formula (I):

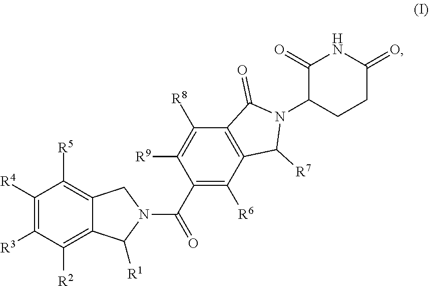

or a pharmaceutically acceptable salt, tautomer, isotopologue, or stereoisomer thereof, wherein:
R¹ is (C₁-C₃)alkyl or —CH₂N(R¹⁰)₂;
   each R¹⁰ is independently (C₁-C₃)alkyl or two R¹⁰, together with the nitrogen to which they are attached, form a three- to eight-membered heterocyclyl optionally substituted with (R¹¹)ₓ;
   each R¹¹ is independently halo, (C₁-C₃)alkyl, or halo(C₁-C₃)alkyl;
   x is 1, 2, 3, 4, or 5;
R² is H, halo, (C₁-C₃)alkyl, halo(C₁-C₃)alkyl, —O—(C₁-C₃)alkyl, or (C₃-C₅)cycloalkyl;
R³ is H, halo, (C₁-C₃)alkyl, halo(C₁-C₃)alkyl, —O—(C₁-C₃)alkyl, or (C₃-C₅)cycloalkyl;
R⁴ is H, halo, (C₁-C₃)alkyl, halo(C₁-C₃)alkyl, —O—(C₁-C₃)alkyl, or (C₃-C₅)cycloalkyl;
R⁵ is H, halo, (C₁-C₃)alkyl, halo(C₁-C₃)alkyl, —O—(C₁-C₃)alkyl, or (C₃-C₅)cycloalkyl;
R⁶ is H, halo, (C₁-C₃)alkyl, halo(C₁-C₃)alkyl, or —O—(C₁-C₃)alkyl; and
R⁷ is H or (C₁-C₃)alkyl; or
R⁶ and R⁷, taken together with their intervening atoms, form a 5-7-membered cycle; and
R⁸ and R⁹ are each independently H or halo.

Also provided herein is a compound of structural formula (Ia):

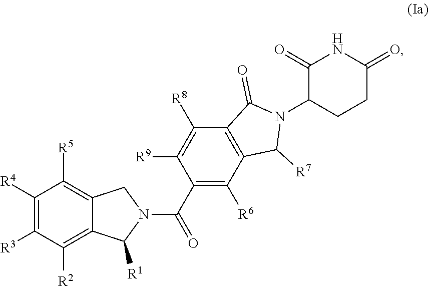

or a pharmaceutically acceptable salt, tautomer, or isotopologue thereof, wherein values for the variables (e.g., R¹, R², R³, R⁴, R⁵, R⁶, R⁷, R⁸, R⁹) are as described herein, e.g., with respect to structural formula (I).

Also provided herein is a compound of structural formula (Ib):

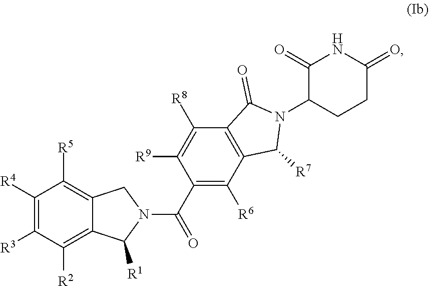

or a pharmaceutically acceptable salt, tautomer, or isotopologue thereof, wherein values for the variables (e.g., R¹, R², R³, R⁴, R⁵, R⁶, R⁷, R⁸, R⁹) are as described herein, e.g., with respect to structural formula (I).

Also provided herein is a compound of structural formula (Ic):

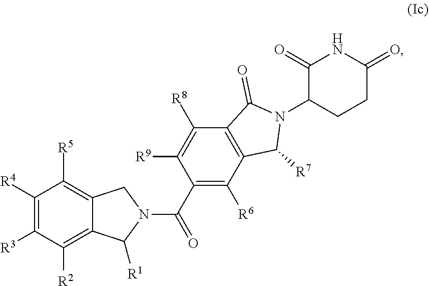

or a pharmaceutically acceptable salt, tautomer, or isotopologue thereof, wherein values for the variables (e.g., R¹, R², R³, R⁴, R⁵, R⁶, R⁷, R⁸, R⁹) are as described herein, e.g., with respect to structural formula (I).

Also provided herein is a compound of structural formula (Id):

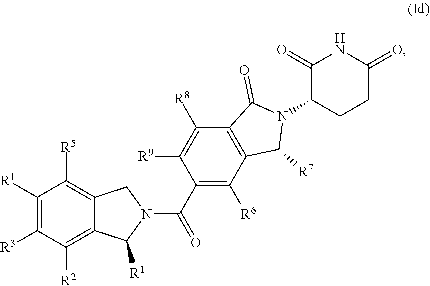

or a pharmaceutically acceptable salt, tautomer, or isotopologue thereof, wherein values for the variables (e.g., R¹, R², R³, R⁴, R⁵, R⁶, R⁷, R⁸, R⁹) are as described herein, e.g., with respect to structural formula (I).

In some embodiments:
R¹ is (C₁-C₃)alkyl or —CH₂N(R¹⁰)₂;
each R¹⁰ is independently (C₁-C₃)alkyl or two R¹⁰, together with the nitrogen to which they are attached, form a four- to eight-membered heterocyclyl optionally substituted with (R¹¹)ₓ;
each R¹¹ is independently halo, (C₁-C₃)alkyl, or halo(C₁-C₃)alkyl; and
x is 1, 2, 3, 4, or 5

In some embodiments, R¹ is (C₁-C₃)alkyl. In some embodiments, R¹ is methyl.

In some embodiments, R¹ is —CH₂N(R¹⁰)₂. In some embodiments, R¹ is —CH₂N(CH₃)₂. In some embodiments, R¹ is

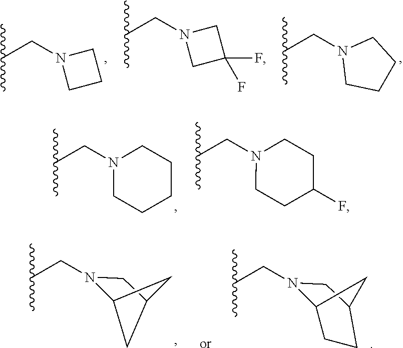

In some embodiments, R¹ is —CH₃, —CH₂N(CH₃)₂,

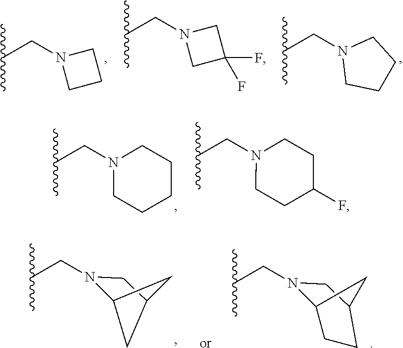

In some embodiments, R¹ is —CH₃ or —CH₂N(CH₃)₂.

In some embodiments, R² is H, halo, (C₁-C₃)alkyl, or —O—(C₁-C₃)alkyl. In some embodiments, R² is H or halo. In some embodiments, R² is H or F. In some embodiments, R² is H. In some embodiments, R² is F. In some embodiments, R² is halo.

In some embodiments, R³ is H or halo (e.g., F, Cl, or Br). In some embodiments, R³ is H or F. In some embodiments, R³ is H. In some embodiments, R³ is F. In some embodiments, R³ is halo (e.g., F, Cl, or Br).

In some embodiments, R² is H and R³ is H. In some embodiments, R² is H and R³ is halo (e.g., F, Cl, or Br). In some embodiments, R² is halo and R³ is H. In some embodiments, R² is H and R³ is F. In some embodiments, R² is F and R³ is H.

In some embodiments, R⁴ is H, F, Cl, Br, methyl, trifluoromethyl, methoxy, ethoxy, or cyclopropyl. In some embodiments, R⁴ is H, halo, (C₁-C₃)alkyl, or halo(C₁-C₃)alkyl. In some embodiments, R⁴ is H, F, Cl, Br, methyl or trifluoromethyl.

In some embodiments, R⁵ is H, halo, (C₁-C₃)alkyl, or —O—(C₁-C₃)alkyl. In some embodiments, R⁵ is H, F, Cl, Br, methyl, or methoxy. In some embodiments, R⁵ is H or halo. In some embodiments, R⁵ is H, F, or Br.

In some embodiments, R⁴ is halo or halo(C₁-C₃)alkyl and R⁵ is H. In some embodiments, R⁴ is halo(C₁-C₃)alkyl and R⁵ is H. In some embodiments, R⁴ is trifluoromethyl and R⁵ is H. In some embodiments, R⁴ is H, halo, or (C₁-C₃)alkyl, and R⁵ is halo. In some embodiments, R⁴ is H, F, Cl, Br, or methyl, and R⁵ is halo. In some embodiments, R⁴ is H, halo, or (C₁-C₃)alkyl, and R⁵ is F or Br. In some embodiments, R⁴ is H, F, Cl, Br, or methyl, and R⁵ is F or Br. In some embodiments, R⁴ and R⁵ are each independently halo.

In some embodiments, R⁶ is H, halo, (C₁-C₃)alkyl, halo(C₁-C₃)alkyl, or —O—(C₁-C₃)alkyl; and R⁷ is H or (C₁-C₃)alkyl. In further embodiments, R⁶ is H, F, or methoxy; and R⁷ is H or (C₁-C₃)alkyl. In yet further embodiments, R⁶ is H; and R⁷ is H or (C₁-C₃)alkyl. In some embodiments, R⁶ is H, halo, (C₁-C₃)alkyl, halo(C₁-C₃)alkyl, or —O—(C₁-C₃)alkyl; and R⁷ is H or methyl. In further embodiments, R⁶ is H, halo, (C₁-C₃)alkyl, halo(C₁-C₃)alkyl, or —O—(C₁-C₃)alkyl; and R⁷ is methyl. In some embodiments, R⁶ is H, F, or methoxy; and R⁷ is H or methyl. In further embodiments, R⁶ is H; and R⁷ is methyl.

In some embodiments, R⁶ and R⁷, taken together with their intervening atoms, form a 5-7-membered cycle. In some embodiments, R⁶ and R⁷, taken together with their intervening atoms, form a 5- or 6-membered aromatic or heteroaromatic cycle. In some embodiments, R⁶ and R⁷, taken together with their intervening atoms, form benzene or pyridine. In some embodiments, R⁶ and R⁷, taken together with their intervening atoms, form benzene. In some embodiments, R⁶ and R⁷, taken together with their intervening atoms, form benzene, pyridine, or tetrahydropyran.

In some embodiments, R⁸ and R⁹ are each independently H or F. In some embodiments, R⁸ and R⁹ are both H. In some embodiments, R⁸ and R⁹ are both F. In some embodiments, one of R⁸ and R⁹ is H, and the other is F. In some embodiments, R⁸ is H and R⁹ is F. In some embodiments, R⁸ is F and R⁹ is H. In some embodiments, one of R⁸ and R⁹ is H, and the other is halo. In some embodiments, R⁸ is H and R⁹ is halo. In some embodiments, R⁸ is halo and R⁹ is H.

In some embodiments, each R¹⁰ is independently (C₁-C₃)alkyl. In some embodiments, each R¹⁰ is methyl. In alternative embodiments, two R¹⁰, together with the nitrogen to which they are attached, form a three- to eight-membered heterocyclyl optionally substituted with (R¹¹)ₓ. In some embodiments, two R¹⁰, together with the nitrogen to which they are attached, form a four- to eight-membered heterocyclyl optionally substituted with (R¹¹)ₓ. In some embodiments, two R¹⁰, together with the nitrogen to which they are attached, form a four- to seven-membered heterocyclyl optionally substituted with (R¹¹)ₓ. In some embodiments, two R¹⁰, together with the nitrogen to which they are attached, form pyrrolidinyl, azabicyclo[2.1.1]hexanyl, azabicyclo[2.2.1]heptanyl, piperidinyl, or azetidinyl, optionally substituted with (R¹¹)ₓ.

In some embodiments, each R¹¹ is independently halo, (C₁-C₃)alkyl, or fluoro(C₁-C₃)alkyl. In some embodiments, each R¹¹ is independently fluoro, methyl, trifluoromethyl, or difluoromethyl. In some embodiments, each R¹¹ is independently fluoro, methyl, or trifluoromethyl. In some embodiments, each R¹¹ is independently halo. In some embodiments, each R¹¹ is fluoro.

In some embodiments, x is 1, 2, 3, or 4. In some embodiments, x is 1, 2, or 3. In some embodiments, x is 1 or 2. In some embodiments, x is 1.

Representative compounds of structural formulas (I), (Ia), (Ib), (Ic), and (Id) are set forth in Table 1. In some embodiments, provided herein is one or more compounds of Table 1, or a pharmaceutically acceptable salt thereof.

### Pharmaceutical Compositions and Routes of Administration

Also provided herein are pharmaceutical compositions comprising a compound of the present disclosure (e.g., a compound of structural formula (I), (Ia), (Ib), (Ic) and (Id), of Table 1, or a pharmaceutically acceptable salt, tautomer, isotopologue, or stereoisomer thereof), and a pharmaceutically acceptable carrier, excipient or vehicle.

The compounds described herein can be administered to a subject enterally (for example, orally, rectally), topically, or parenterally (for example, intravenously, intramuscularly, subcutaneously), e.g., in the conventional form of medicinal preparations, such as capsules, microcapsules, tablets, granules, powder, troches, pills, suppositories, injections, suspensions, syrups, patches, creams, lotions, ointments, gels, sprays, solutions and emulsions.

Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrrolidone or aluminum stearate), a dispersing agent (e.g., hydroxypropylmethylcellulose), a diluent (e.g., water), a cosolvent (e.g., propylene glocyl/glycofurol), a buffer, a copolymer (e.g., poly(lactic-co-glycolic acid, i.e PLGA), and/or base wax (e.g., cocoa butter, white petrolatum or polyethylene glycol). The amount of a compound in a pharmaceutical composition may be at a level that will exert a desired effect, for example, in unit a dosage for oral or parenteral administration.

The dose of a compound to be administered to a subject is rather widely variable and can be subject to the judgment of a health-care practitioner. In general, the compounds described herein can be administered one to four times per day at a dose that may depend, for example, on the age, body weight, and medical condition of the subject and/or the route of administration. In any given case, the amount of the compound administered will depend on such factors as the solubility of the active component, the formulation used, and the route of administration.

A compound of the present disclosure can be administered once, twice, three, four or more times daily. For example, the daily dose may be divided into multiple doses in amounts that together add up to the total daily dose.

A compound of the present disclosure can be administered orally. In one embodiment, when administered orally, the compound is administered with a meal and water. In another embodiment, the compound is dispersed in water or juice (e.g., apple juice or orange juice) and administered orally as a suspension.

A compound of the present disclosure can also or alternatively be administered intradermally, intramuscularly, intraperitoneally, percutaneously, intravenously, subcutaneously, intranasally, epidurally, sublingually, intracerebrally, intravaginally, transdermally, rectally, mucosally, by inhalation, topically to the ears, nose, eyes, or skin, or by local ocular (i.e., subconjunctival, intravitreal, retrobulbar, intracameral) administration.

Also provided herein are unit dosage forms of a compound of the present disclosure (e.g., a compound of structural formula (I), (Ia), (Ib), (Ic), and (Id), of Table 1, or a pharmaceutically acceptable salt, tautomer, isotopologue, or stereoisomer thereof).

In one embodiment, provided herein are capsules containing a compound of the present disclosure without an additional carrier, excipient or vehicle.

Pharmaceutical compositions can be in the form of tablets (e.g., chewable tablets), capsules, solutions (e.g., parenteral solutions), troches, suppositories, suspensions, gels, intra-ruminal devices (e.g., for prolonged prophylaxis or controlled release), implants, topical pour-ons, transdermal delivery gels, spot-ons, implants (including devices, gels, liquids (e.g., PLGA)), and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts. In general, all of the compositions are prepared according to known methods in pharmaceutical chemistry.

For example, capsules can be prepared by mixing a compound of the present disclosure with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound of the present disclosure. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

The effect of a compound of the present disclosure can be delayed or prolonged by proper formulation. For example, a slowly soluble pellet of a compound of the present disclosure can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, e.g., by dissolving or suspending a compound of the present disclosure in oily or emulsified vehicles, or adding amounts of PLGA, that allow it to disperse slowly in the serum.

### Methods of Use

Certain compounds of the present disclosure are shown herein to modulate (e.g., inhibit) ARNT activity, e.g., as by inducing degradation of ARNT.

Accordingly, provided herein are methods of modulating ARNT activity in a cell (e.g., a cell expressing ARNT), comprising contacting the cell with a compound of the present disclosure (e.g., a compound of structural formula (I), (Ia), (Ib), (Ic), and (Id), or Table 1, or a pharmaceutically acceptable salt, tautomer, isotopologue, or stereoisomer thereof). In some embodiments, modulating ARNT activity comprises inhibiting ARNT activity, e.g., as a compound that is an ARNT antagonist might. In some embodiments, modulating ARNT activity comprises reducing a level of ARNT in the cell as, for example, by inducing degradation of ARNT. In some embodiments, the method is a method of inducing degradation of ARNT in a cell (e.g., a cell expressing ARNT).

In some embodiments, the cell is in vitro. In some embodiments, the cell is ex vivo. In some embodiments, the cell is in vivo. In some embodiments, the cell is in a subject (e.g., a subject in need thereof, such as a subject having a cancer, e.g., renal cell carcinoma).

Also provided herein are methods of modulating ARNT activity (e.g., inducing degradation of ARNT) in a subject in need thereof, comprising administering to the subject a therapeutically effective amount of a compound of the present disclosure (e.g., a compound of structural formula (I), (Ia), (Ib), (Ic), and (Id), or Table 1, or a pharmaceutically acceptable salt, tautomer, isotopologue, or stereoisomer thereof).

Also provided herein are methods of treating cancer in a subject in need thereof, comprising administering to the subject a therapeutically effective amount of a compound of the present disclosure (e.g., a compound of structural formula (I), (Ia), (Ib), (Ic), and (Id), or Table 1, or a pharmaceutically acceptable salt, tautomer, isotopologue, or stereoisomer thereof). In some embodiments, the cancer is a solid tumor cancer. In some embodiments, the cancer is a hematologic cancer. In some embodiments, the cancer is renal cell carcinoma. In some embodiments, the renal cell carcinoma is clear cell renal cell carcinoma (ccRCC).

Compounds of the present disclosure can be administered to a subject in accordance with the methods described herein via any of the routes of administration described herein.

### EXAMPLES

The following Examples are presented by way of illustration, not limitation. One skilled in the art can modify the procedures set forth in the illustrative examples to arrive at the desired products.

Salts of the compounds described herein can be prepared by standard methods, such as by including an acid, for example TFA, formic acid, or HCl, in the mobile phases during chromatography purification, or by stirring of the products after chromatography purification with a solution of an acid, for example, aqueous HCl.

All reactions are at room temperature unless noted otherwise.

The following abbreviations may be relevant for the application:

| | |
|---|---|
| Boc | tert-butyloxycarbonyl |
| AIBN | azobisisobutyronitrile |
| ACN | acetonitrile |
| BSA | bis(trimethylsilyl)acetamide |
| DCE | 1,2-dichloroethane |
| DCM | dichloromethane |
| DIEA | N,N-diisopropylethylamine |
| DIPEA | N,N-diisopropylethylamine |
| DME | dimethyl ether |
| DMF | dimethyl formamide |
| DMSO | dimethyl sulfoxide |
| DTBAD | di-tert-butyl azodicarboxylate |
| Dtbbpy | 4,4′-di-tert-butyl-2,2′-bipyridine |
| equiv | equivalent |
| ESI | electrospray ionization |
| EtOH | ethanol |
| FA | formic acid |
| h | hour |
| HATU | O-(7-azabenzotriazol-1-yl)-N,N,N′,N′-tetramethyluronium |
| | hexafluorophosphate |
| HCl | hydrochloric acid |
| HOAc | Acetic acid |
| HPLC | high performance liquid chromatography |
| LAH | lithium aluminum hydride |
| LED | light-emitting diode |
| LiOtBu | lithium tert-butoxide |
| MeCN | acetonitrile |
| MeOH | methanol |
| min | minute |
| MS | mass spectrometry |
| NBS | N-bromo succinimide |
| NMO | 4-methylmorpholine 4-oxide |
| NMP | N-methyl-2-pyrrolidone |
| NMR | nuclear magnetic resonance |
| PivOH | pivalic acid |
| PMBCl | 4-methoxybenzyl chloride |
| Py | pyridine |
| rt | room temperature |
| RT | retention time |
| sat. | saturated |
| t | time |
| t-BuOH | tert-butyl alcohol |
| t-BuOLi | lithium tert-butoxide |
| TEA | triethylamine |
| TFA | trifluoroacetic acid |
| TfOH | triflic acid |
| THF | tetrahydrofuran |
| TLC | thin layer chromatography |
| TMP | 2,2,6,6-tetramethylpiperidine |

### Materials and Methods (LC-MS)

Chemicals: All reagents used were analytical or ultra gradient grade.

Sample preparation: Powders were solubilized in DMSO, methanol, acetonitrile, or water, or in another solvent depending on their solubility, at the concentration around 0.5 mg/mL, vortexed vigorously until the solution was clear and transferred to a LCMS vial for data acquisition.

LCMS analysis: Two different LC-UV-MS systems were used:
1. Waters Acquity UPLC system equipped with Waters Acquity PDA e-detector coupled with a Waters simple quadruple detector SQD mass spectrometer; and
2. Waters HClass equipped with a UPLC eLAMBDA PDA detector coupled with a Waters simple quadruple detector SQD mass spectrometer.

System 1 was configured in acidic reverse phase conditions. Chromatographic separations were performed with:
Waters column Acquity UPLC® CSH™ C18 (2.1×50 mm) 1.7 μm−Eluent A=H₂O+0.02% HCOOH−Eluent B=CH₃CN+0.02% HCOOH−Oven Temperature (T, ° C.): 55° C.−Gradient: t0 2% B, t4 min 98% B, t4.5 min 98% B, t4.6 min 2% B, t5.0 min 2% B−Flow rate=1 mL/min; or
Waters column Acquity UPLC® CSH™ C18 (2.1×100 mm) 1.7 μm−Eluent A=H₂O+0.02% HCOOH−Eluent B=CH₃CN+0.02% HCOOH−Oven T (° C.): 55° C.−Gradient: t0 2% B, t15 min 98% B, t15.2 min 2% B, t18 min 2% B−Flow rate=0.7 mL/min.

The injection volume was set from 0.5 μL to 2 μL. The UV chromatograms were recorded at a wavelength of 220 nm.

The mass spectrometer was operated with a source configured in positive or negative electrospray ion mode. The source parameters were: source temperature 150° C.; desolvatation temperature 250° C.; cone gas flow 150 (L/Hr); desolvatation gas flow 1000 (L/Hr); Capillary: 3 kV—Sample cone: 15/30V—Mass range: 120-1500 amu.

Data acquisition and data reprocessing were performed using Masslynx software (Waters).

System 2 was configured in acid reverse phase conditions, neutral reverse phase conditions, or basic reverse phase conditions. Chromatographic separations were performed as follows:
Waters Column Acquity UPLC CSH C18 (2.1×50 mm) 1.7 μm−Eluent A=H₂O+0.02% HCOOH−Eluent B=CH₃CN+0.02% HCOOH−Oven T (° C.): 55° C.−Gradient: t0 2% B, t4 min 98% B, t4.5 min 98% B, t4.6 min 2% B, t5.5 min 2% B−Flow rate=1 mL/min; or
Waters Column Acquity UPLC CSH C18 (2.1×100 mm) 1.7 μm−Eluent A=H₂O+0.02% HCOOH−Eluent B=CH₃CN+0.02% HCOOH−Oven T (° C.): 55° C.−Gradient: t0 2% B, t15 min 98% B, t15.2 min 2% B, t18 min 2% B−Flow rate=0.7 mL/min; or
Waters Column Acquity UPLC CSH C18 (2.1×50 mm) 1.7 μm−Eluent A=H₂O+0.05% TFA−Eluent B=CH₃CN+0.035% TFA−Oven T (° C.): 55° C.−Gradient: t0 2% B, t4 min 98% B, t4.5 min 98% B, t4.6 min 2% B, t5.5 min 2% B−Flow rate=1 mL/min; or
Waters Column Acquity UPLC CSH C18 (2.1×100 mm) 1.7 μm−Eluent A=H₂O+0.05% TFA−Eluent B=CH₃CN+0.035% TFA−Oven T (° C.): 55° C.−Gradient: t0 2% B, t15 min 98% B, t15.2 min 2% B, t18 min 2% B−Flow rate=0.7 mL/min; or
Waters Column Acquity UPLC BEH C18 (2.1×50 mm) 1.7 μm−Eluent A=H₂O+AcONH₄ 10 mM (ammoniac in water adjusted to pH 7 with acetic acid)−Eluent B=CH₃CN -Oven T (° C.): 45° C.−Gradient: t0 2% B, t4 min 90% B, t4.5 min 90% B, t4.6 min 2% B, t5.5 min 2% B−Flow rate=0.8 mL/min; or
Waters Column Acquity UPLC BEH C18 (2.1×50 mm) 1.7 μm−Eluent A=H₂O+NH₄HCO₃ at 2 mM adjusted to pH 10 with NaOH−Eluent B=CH₃CN−Oven T (° C.): 45° C. -Gradient: t0 2% B, t4 min 90% B, t4.5 min 90% B, t4.6 min 2% B, t5.5 min 2% B−Flow rate=0.8 mL/min.

The injection volume was set from 0.5 μL to 2 μL. The UV chromatograms were recorded at a wavelength of 220 nm.

The mass spectrometer was operated with a source configured in positive or negative electrospray ion mode. The source parameters were: source temperature 150° C.; desolvatation temperature 250° C.; cone gas flow 150 (L/Hr); desolvatation gas flow 1000 (L/Hr); Capillary: 3 kV—Sample cone: 15/30V—Mass range: 120-1500 amu.

Data acquisition and data reprocessing were performed using Masslynx software (Waters).

### Materials and Methods (NMR)

Sample preparation: Powders were solubilized in DMSO-d6, vortexed vigorously until the solution was clear, and transferred to a NMR tube for data acquisition.

NMR spectroscopy: Liquid-state NMR experiments were recorded on a 600 MHz (14.1 Tesla) Bruker Avance III NMR spectrometer (600 MHz for ¹H, 151 MHz for ¹³C) using a triple-resonance ¹H, ¹⁹F, ¹H, ¹⁵N, ¹³C TR TCI 5 mm cryoprobe (Bruker Biospin, Germany); a 500 MHz (11.75 Tesla) Bruker Avance NEO NMR spectrometer (500 MHz for ¹H, 125 MHz for ¹³C) using a dual resonance 5 mm BBFO BBF/H iProbe (Bruker Biospin, Germany); and/or a 400 MHz (9.4 Tesla) Bruker Avance NEO NMR spectrometer (400 MHz for ¹H, 100 MHz for ¹³C) using a SEI 5 mm probe (Bruker Biospin, Germany).

All the experiments used for the resonance assignment procedure and the elucidation of the products structure (e.g., one-dimensional (1D) ¹H, two-dimensional (2D)¹H-¹H-COSY, 2D ¹H-¹H-ROESY, 2D ¹H-¹³C-HSQC, 2D ¹H-¹³C-HMBC) were recorded at 300K. ¹H chemical shifts are reported in d (ppm) as s (singlet), d (doublet), t (triplet), q (quartet), dd (double doublet), m (multiplet) or br s (broad singlet).

As will be apparent to one skilled in the art, the compounds disclosed below and in Table 1 can exist in various stereochemical forms.

TABLE 1

| Cmpd. | | |
|---|---|---|
| No. | Compound Structure | Compound Name |
| 1 | 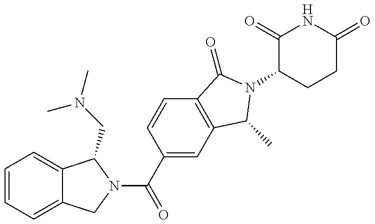 [SMILES: C[C@@H]1c2cc(C(=O)N3Cc4ccccc4[C@@H]3CN(C)C)ccc2C(=O)N1[C@H]1CCC(=O)NC1=O] | (S)-3-((R)-5-((R)-1- ((dimethylamino)methyl)isoin- doline-2-carbonyl)-3-methyl- 1-oxoisoindolin-2- yl)piperidine-2,6-dione |
| 2 | 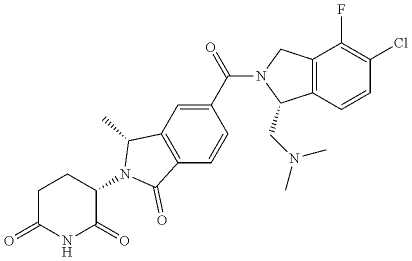 [SMILES: C[C@@H]1c2cc(C(=O)N3Cc4c(ccc(Cl)c4F)[C@@H]3CN(C)C)ccc2C(=O)N1[C@H]1CCC(=O)NC1=O] | (S)-3-((R)-5-((R)-5-chloro-1- ((dimethylamino)methyl)-4- fluoroisoindoline-2-carbonyl)- 3-methyl-1-oxoisoindolin-2- yl)piperidine-2,6-dione |
| 3 | 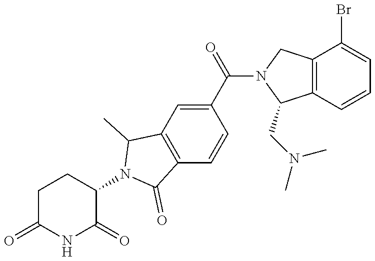 [SMILES: CC1c2cc(C(=O)N3Cc4c(Br)cccc4[C@@H]3CN(C)C)ccc2C(=O)N1[C@H]1CCC(=O)NC1=O] | (S)-3-((R)-5-((R)-4-bromo-1- ((dimethylamino)methyl)isoin- doline-2-carbonyl)-3-methyl- 1-oxoisoindolin-2- yl)piperidine-2,6-dione |
| 4 | 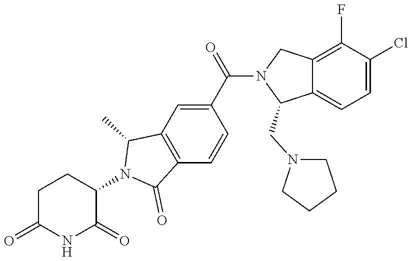 [SMILES: C[C@@H]1c2cc(C(=O)N3Cc4c(ccc(Cl)c4F)[C@@H]3CN3CCCC3)ccc2C(=O)N1[C@H]1CCC(=O)NC1=O] | (S)-3-((R)-5-((R)-5-chloro-4- fluoro-1-(pyrrolidin-1- ylmethyl)isoindoline-2- carbonyl)-3-methyl-1- oxoisoindolin-2-yl)piperidine- 2,6-dione |
| 5 | 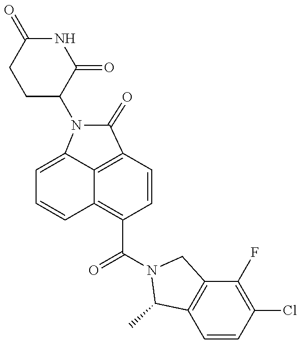 [SMILES: C[C@H]1c2ccc(Cl)c(F)c2CN1C(=O)c1ccc2c3c(cccc13)N(C1CCC(=O)NC1=O)C2=O] | 3-(5-((S)-5-chloro-4-fluoro-1- methylisoindoline-2-carbonyl)- 2-oxobenzo[cd]indol-1(2H)- yl)piperidine-2,6-dione |
| 6 | 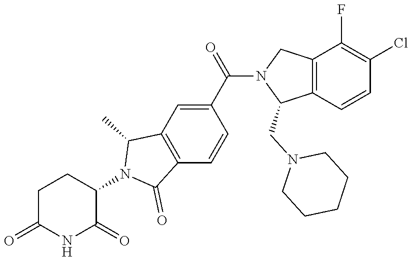 [SMILES: C[C@@H]1c2cc(C(=O)N3Cc4c(ccc(Cl)c4F)[C@@H]3CN3CCCCC3)ccc2C(=O)N1[C@H]1CCC(=O)NC1=O] | (S)-3-((R)-5-((R)-5-chloro-4- fluoro-1-(piperidin-1- ylmethyl)isoindoline-2- carbonyl)-3-methyl-1- oxoisoindolin-2-yl)piperidine- 2,6-dione |
| 7 | 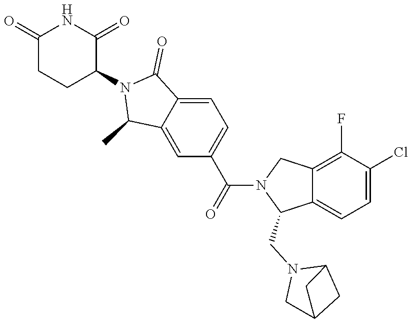 [SMILES: C[C@@H]1c2cc(C(=O)N3Cc4c(ccc(Cl)c4F)[C@@H]3CN3CC4CC3C4)ccc2C(=O)N1[C@H]1CCC(=O)NC1=O] | (S)-3-((R)-5-((R)-1-((2- azabicyclo[2.1.1]hexan-2- yl)methyl)-5-chloro-4- fluoroisoindoline-2-carbonyl)- 3-methyl-1-oxoisoindolin-2- yl)piperidine-2,6-dione |
| 8 | 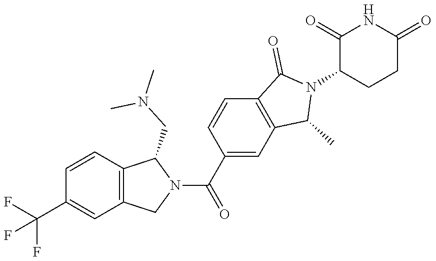 [SMILES: C[C@@H]1c2cc(C(=O)N3Cc4cc(C(F)(F)F)ccc4[C@@H]3CN(C)C)ccc2C(=O)N1[C@H]1CCC(=O)NC1=O] | (S)-3-((R)-5-((R)-1- ((dimethylamino)methyl)-5- (trifluoromethyl)isoindoline-2- carbonyl)-3-methyl-1- oxoisoindolin-2-yl)piperidine- 2,6-dione |
| 9 | 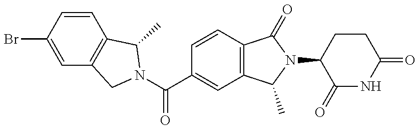 [SMILES: C[C@@H]1c2cc(C(=O)N3Cc4cc(Br)ccc4[C@@H]3C)ccc2C(=O)N1[C@H]1CCC(=O)NC1=O] | (S)-3-((R)-5-((S)-5-bromo-1- methylisoindoline-2-carbonyl)- 3-methyl-1-oxoisoindolin-2- yl)piperidine-2,6-dione |
| 10 | 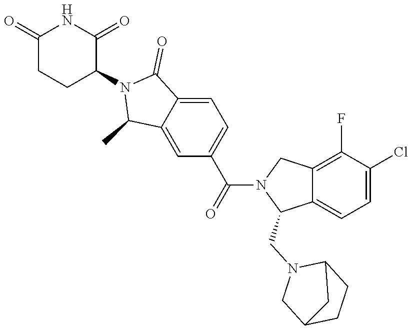 [SMILES: C[C@@H]1c2cc(C(=O)N3Cc4c(ccc(Cl)c4F)[C@@H]3CN3CC4CCC3C4)ccc2C(=O)N1[C@H]1CCC(=O)NC1=O] | (3S)-3-((3R)-5-((1R)-1-((2- azabicyclo[2.2.1]heptan-2- yl)methyl)-5-chloro-4- fluoroisoindoline-2-carbonyl)- 3-methyl-1-oxoisoindolin-2- yl)piperidine-2,6-dione |
| 11 | 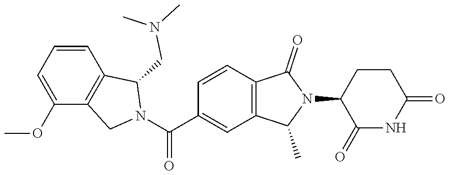 [SMILES: COc1cccc2c1CN(C(=O)c1ccc3c(c1)[C@@H](C)N([C@H]1CCC(=O)NC1=O)C3=O)[C@H]2CN(C)C] | (S)-3-((R)-5-((R)-1- ((dimethylamino)methyl)-4- methoxyisoindoline-2- carbonyl)-3-methyl-1- oxoisoindolin-2-yl)piperidine- 2,6-dione |
| 12 | 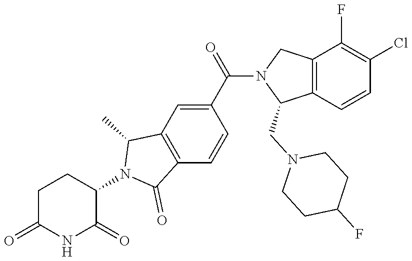 [SMILES: C[C@@H]1c2cc(C(=O)N3Cc4c(ccc(Cl)c4F)[C@@H]3CN3CCC(F)CC3)ccc2C(=O)N1[C@H]1CCC(=O)NC1=O] | (S)-3-((R)-5-((R)-5-chloro-4- fluoro-1-((4-fluoropiperidin-1- yl)methyl)isoindoline-2- carbonyl)-3-methyl-1- oxoisoindolin-2-yl)piperidine- 2,6-dione |
| 13 | 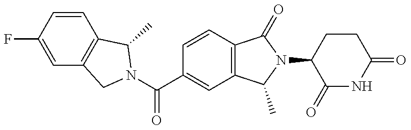 [SMILES: C[C@@H]1c2cc(C(=O)N3Cc4cc(F)ccc4[C@@H]3C)ccc2C(=O)N1[C@H]1CCC(=O)NC1=O] | (S)-3-((R)-5-((S)-5-fluoro-1- methylisoindoline-2-carbonyl)- 3-methyl-1-oxoisoindolin-2- yl)piperidine-2,6-dione |
| 14 | 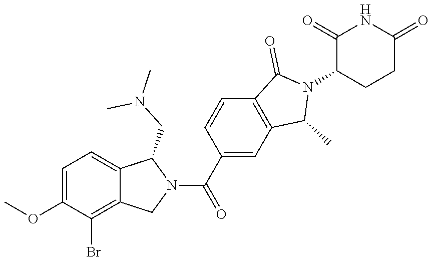 [SMILES: COc1ccc2c(c1Br)CN(C(=O)c1ccc3c(c1)[C@@H](C)N([C@H]1CCC(=O)NC1=O)C3=O)[C@H]2CN(C)C] | (S)-3-((R)-5-((R)-4-bromo-1- ((dimethylamino)methyl)-5- methoxyisoindoline-2- carbonyl)-3-methyl-1- oxoisoindolin-2-yl)piperidine- 2,6-dione |
| 15 | 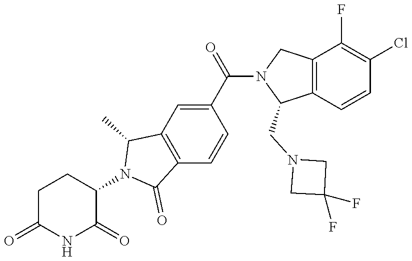 [SMILES: C[C@@H]1c2cc(C(=O)N3Cc4c(ccc(Cl)c4F)[C@@H]3CN3CC(F)(F)C3)ccc2C(=O)N1[C@H]1CCC(=O)NC1=O] | (S)-3-((R)-5-((R)-5-chloro-1- ((3,3-difluoroazetidin-1- yl)methyl)-4- fluoroisoindoline-2-carbonyl)- 3-methyl-1-oxoisoindolin-2- yl)piperidine-2,6-dione |
| 16 | 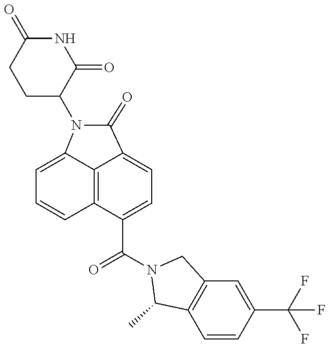 [SMILES: C[C@H]1c2ccc(C(F)(F)F)cc2CN1C(=O)c1ccc2c3c(cccc13)N(C1CCC(=O)NC1=O)C2=O] | 3-(5-((S)-1-methyl-5- (trifluoromethyl)isoindoline-2- carbonyl)-2- oxobenzo[cd]indol-1(2H)- yl)piperidine-2,6-dione |
| 17 | 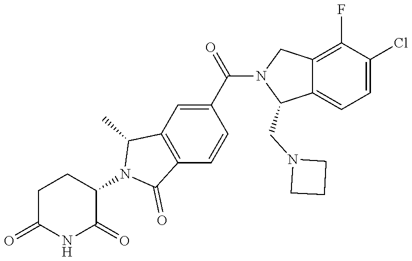 [SMILES: C[C@@H]1c2cc(C(=O)N3Cc4c(ccc(Cl)c4F)[C@@H]3CN3CCC3)ccc2C(=O)N1[C@H]1CCC(=O)NC1=O] | (S)-3-((R)-5-((R)-1-(azetidin- 1-ylmethyl)-5-chloro-4- fluoroisoindoline-2-carbonyl)- 3-methyl-1-oxoisoindolin-2- yl)piperidine-2,6-dione |
| 18 | 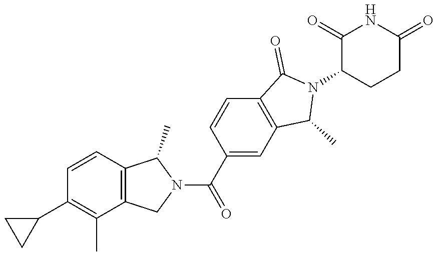 [SMILES: Cc1c(C2CC2)ccc2c1CN(C(=O)c1ccc3c(c1)[C@@H](C)N([C@H]1CCC(=O)NC1=O)C3=O)[C@H]2C] | (S)-3-((R)-5-((S)-5- cyclopropyl-1,4- dimethylisoindoline-2- carbonyl)-3-methyl-1- oxoisoindolin-2-yl)piperidine- 2,6-dione |
| 19 | 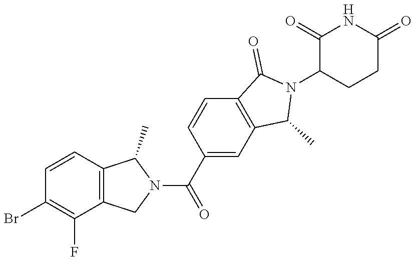 [SMILES: C[C@@H]1c2cc(C(=O)N3Cc4c(ccc(Br)c4F)[C@@H]3C)ccc2C(=O)N1C1CCC(=O)NC1=O] | (S)-3-((R)-5-((S)-5-bromo-4- fluoro-1-methylisoindoline-2- carbonyl)-3-methyl-1- oxoisoindolin-2-yl)piperidine- 2,6-dione |
| 20 | 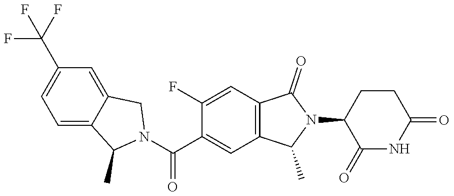 [SMILES: C[C@@H]1c2cc(C(=O)N3Cc4cc(C(F)(F)F)ccc4[C@@H]3C)c(F)cc2C(=O)N1[C@H]1CCC(=O)NC1=O] | (S)-3-((R)-5-fluoro-1-methyl- 6-((S)-1-methyl-5- (trifluoromethyl)isoindoline-2- carbonyl)-3-oxoisoindolin-2- yl)piperidine-2,6-dione |
| 21 | 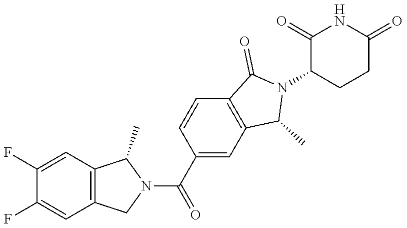 [SMILES: C[C@@H]1c2cc(C(=O)N3Cc4cc(F)c(F)cc4[C@@H]3C)ccc2C(=O)N1[C@H]1CCC(=O)NC1=O] | (S)-3-((R)-5-((S)-5,6-difluoro- 1-methylisoindoline-2- carbonyl)-3-methyl-1- oxoisoindolin-2-yl)piperidine- 2,6-dione |
| 22 | 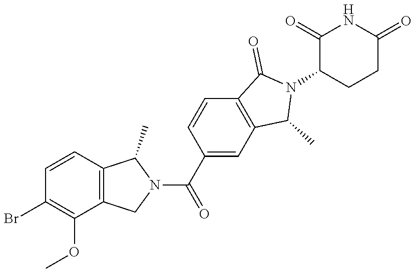 [SMILES: COc1c(Br)ccc2c1CN(C(=O)c1ccc3c(c1)[C@@H](C)N([C@H]1CCC(=O)NC1=O)C3=O)[C@H]2C] | (S)-3-((R)-5-((S)-5-bromo-4- methoxy-1-methylisoindoline- 2-carbonyl)-3-methyl-1- oxoisoindolin-2-yl)piperidine- 2,6-dione |
| 23 | 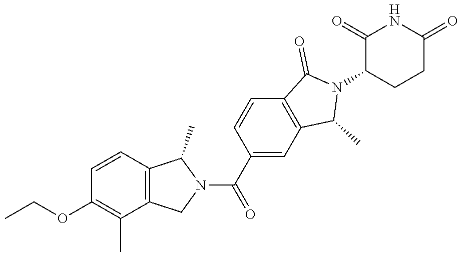 [SMILES: CCOc1ccc2c(c1C)CN(C(=O)c1ccc3c(c1)[C@@H](C)N([C@H]1CCC(=O)NC1=O)C3=O)[C@H]2C] | (S)-3-((R)-5-((S)-5-ethoxy-1,4- dimethylisoindoline-2- carbonyl)-3-methyl-1- oxoisoindolin-2-yl)piperidine- 2,6-dione |
| 24 | 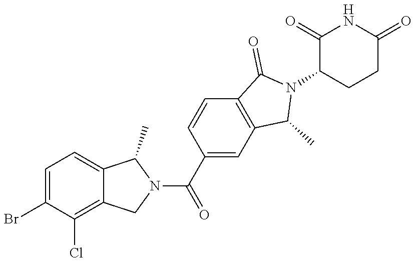 [SMILES: C[C@@H]1c2cc(C(=O)N3Cc4c(ccc(Br)c4Cl)[C@@H]3C)ccc2C(=O)N1[C@H]1CCC(=O)NC1=O] | (S)-3-((R)-5-((S)-5-bromo-4- chloro-1-methylisoindoline-2- carbonyl)-3-methyl-1- oxoisoindolin-2-yl)piperidine- 2,6-dione |
| 25 | 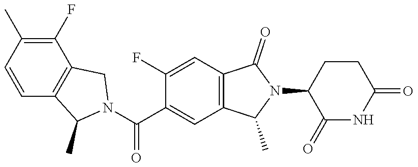 [SMILES: Cc1ccc2c(c1F)CN(C(=O)c1cc3c(cc1F)C(=O)N([C@H]1CCC(=O)NC1=O)[C@@H]3C)[C@H]2C] | (S)-3-((R)-5-fluoro-6-((S)-4- fluoro-1,5- dimethylisoindoline-2- carbonyl)-1-methyl-3- oxoisoindolin-2-yl)piperidine- 2,6-dione |
| 26 | 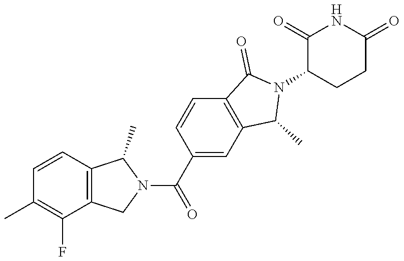 [SMILES: Cc1ccc2c(c1F)CN(C(=O)c1ccc3c(c1)[C@@H](C)N([C@H]1CCC(=O)NC1=O)C3=O)[C@H]2C] | (S)-3-((R)-5-((S)-4-fluoro-1,5- dimethylisoindoline-2- carbonyl)-3-methyl-1- oxoisoindolin-2-yl)piperidine- 2,6-dione |
| 27 | 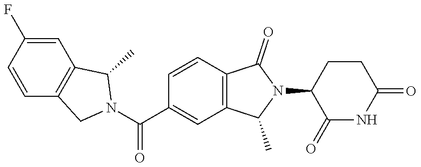 [SMILES: C[C@@H]1c2cc(C(=O)N3Cc4ccc(F)cc4[C@@H]3C)ccc2C(=O)N1[C@H]1CCC(=O)NC1=O] | (S)-3-((R)-5-((S)-6-fluoro-1- methylisoindoline-2-carbonyl)- 3-methyl-1-oxoisoindolin-2- yl)piperidine-2,6-dione |
| 28 | 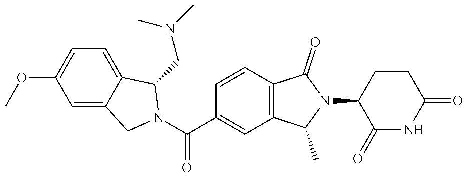 [SMILES: COc1ccc2c(c1)CN(C(=O)c1ccc3c(c1)[C@@H](C)N([C@H]1CCC(=O)NC1=O)C3=O)[C@H]2CN(C)C] | (S)-3-((R)-5-((R)-1- ((dimethylamino)methyl)-5- methoxyisoindoline-2- carbonyl)-3-methyl-1- oxoisoindolin-2-yl)piperidine- 2,6-dione |
| 29 | 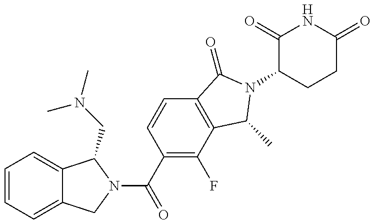 [SMILES: C[C@@H]1c2c(ccc(C(=O)N3Cc4ccccc4[C@@H]3CN(C)C)c2F)C(=O)N1[C@H]1CCC(=O)NC1=O] | (S)-3-((R)-5-((R)-1- ((dimethylamino)methyl)isoin- doline-2-carbonyl)-4-fluoro-3- methyl-1-oxoisoindolin-2- yl)piperidine-2,6-dione |
| 30 | 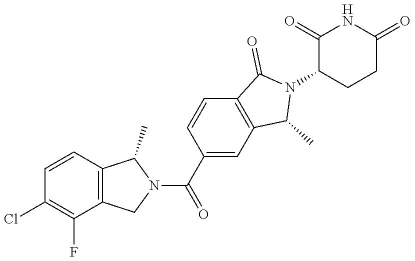 [SMILES: C[C@@H]1c2cc(C(=O)N3Cc4c(ccc(Cl)c4F)[C@@H]3C)ccc2C(=O)N1[C@H]1CCC(=O)NC1=O] | (S)-3-((R)-5-((S)-5-chloro-4- fluoro-1-methylisoindoline-2- carbonyl)-3-methyl-1- oxoisoindolin-2-yl)piperidine- 2,6-dione |
| 31 | 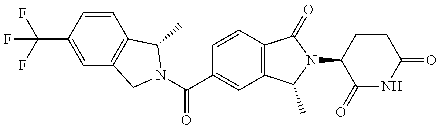 [SMILES: C[C@@H]1c2cc(C(=O)N3Cc4cc(C(F)(F)F)ccc4[C@@H]3C)ccc2C(=O)N1[C@H]1CCC(=O)NC1=O] | (S)-3-((R)-3-methyl-5-((S)-1- methyl-5- (trifluoromethyl)isoindoline-2- carbonyl)-1-oxoisoindolin-2- yl)piperidine-2,6-dione |
| 32 | 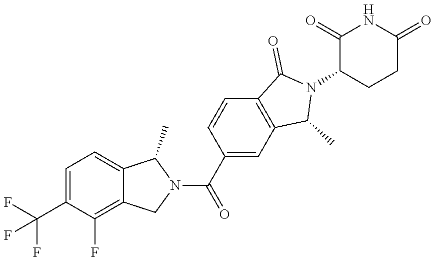 [SMILES: C[C@@H]1c2cc(C(=O)N3Cc4c(ccc(C(F)(F)F)c4F)[C@@H]3C)ccc2C(=O)N1[C@H]1CCC(=O)NC1=O] | (S)-3-((R)-5-((S)-4-fluoro-1- methyl-5- (trifluoromethyl)isoindoline-2- carbonyl)-3-methyl-1- oxoisoindolin-2-yl)piperidine- 2,6-dione |
| 33 | 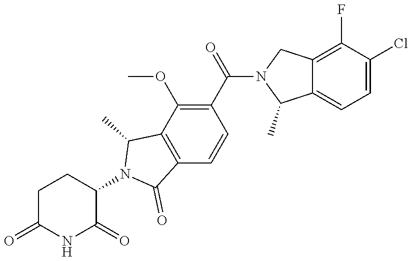 [SMILES: COc1c(C(=O)N2Cc3c(ccc(Cl)c3F)[C@@H]2C)ccc2c1[C@@H](C)N([C@H]1CCC(=O)NC1=O)C2=O] | (S)-3-((R)-5-((S)-5-chloro-4- fluoro-1-methylisoindoline-2- carbonyl)-4-methoxy-3- methyl-1-oxoisoindolin-2- yl)piperidine-2,6-dione |
| 34 | 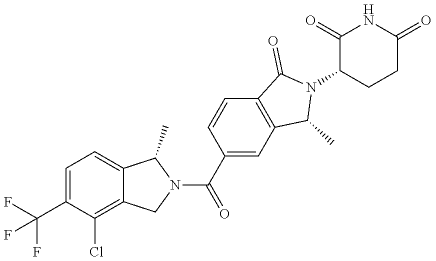 [SMILES: C[C@@H]1c2cc(C(=O)N3Cc4c(ccc(C(F)(F)F)c4Cl)[C@@H]3C)ccc2C(=O)N1[C@H]1CCC(=O)NC1=O] | (S)-3-((R)-5-((S)-4-chloro-1- methyl-5- (trifluoromethyl)isoindoline-2- carbonyl)-3-methyl-1- oxoisoindolin-2-yl)piperidine- 2,6-dione |
| 35 | 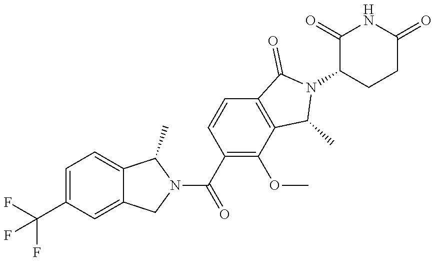 [SMILES: COc1c(C(=O)N2Cc3cc(C(F)(F)F)ccc3[C@@H]2C)ccc2c1[C@@H](C)N([C@H]1CCC(=O)NC1=O)C2=O] | (S)-3-((R)-4-methoxy-3- methyl-5-((S)-1-methyl-5- (trifluoromethyl)isoindoline-2- carbonyl)-1-oxoisoindolin-2- yl)piperidine-2,6-dione |
| 36 | 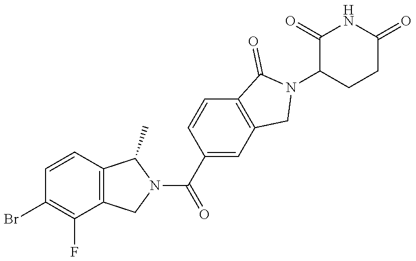 [SMILES: C[C@H]1c2ccc(Br)c(F)c2CN1C(=O)c1ccc2c(c1)CN(C1CCC(=O)NC1=O)C2=O] | 3-(5-((S)-5-bromo-4-fluoro-1- methylisoindoline-2-carbonyl)- 1-oxoisoindolin-2- yl)piperidine-2,6-dione |
| 37 | 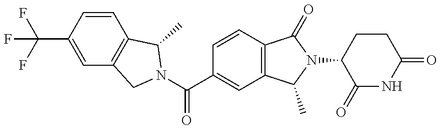 [SMILES: C[C@@H]1c2cc(C(=O)N3Cc4cc(C(F)(F)F)ccc4[C@@H]3C)ccc2C(=O)N1[C@@H]1CCC(=O)NC1=O] | (R)-3-((R)-3-methyl-5-((S)-1- methyl-5- (trifluoromethyl)isoindoline-2- carbonyl)-1-oxoisoindolin-2- yl)piperidine-2,6-dione |
| 38 | 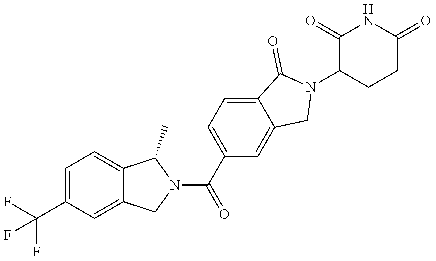 [SMILES: C[C@H]1c2ccc(C(F)(F)F)cc2CN1C(=O)c1ccc2c(c1)CN(C1CCC(=O)NC1=O)C2=O] | 3-(5-((S)-1-methyl-5- (trifluoromethyl)isoindoline-2- carbonyl)-1-oxoisoindolin-2- yl)piperidine-2,6-dione |

### Example 1: Synthesis of CRBN Cores 1, 2, 3, 4, 5, and 6

### Example 1a: CRBN Core 1 Synthesis

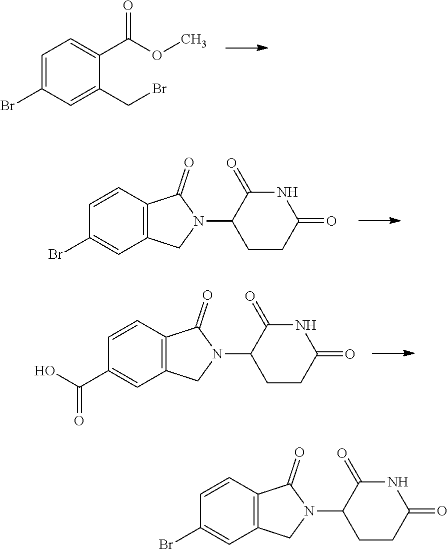

### 3-(5-bromo-1-oxo-isoindolin-2-yl)piperidine-2,6-dione

To a solution of methyl 4-bromo-2-(bromomethyl)benzoate (5.0 g, 15.4 mmol) in DMF (20 mL) were added 3-aminopiperidine-2,6-dione hydrochloride (2.9 g, 17.0 mmol) and N-ethyl-N-(propan-2-yl)propan-2-amine (8.1 mL, 46.3 mmol). The reaction mixture was stirred at 50° C. for 7 hours. Acetic acid (8.8 mL, 0.154 mol) was added and the reaction mixture was stirred for 16 hours at 50° C. The reaction mixture was cooled down to ambient temperature, diluted with water (100 mL) and cooled down to 10° C. The mixture was filtered, the solid was washed with EtOAc (2×40 ml) and dried under reduced pressure to afford 3-(5-bromo-1-oxo-isoindolin-2-yl)piperidine-2,6-dione (4.5 g, 89% yield) as a white solid. ¹H NMR (400 MHz, DMSO) δ 11.00 (s, 1H), 7.90 (d, J=1.7 Hz, 1H), 7.72 (dd, J=8.2, 1.7 Hz, 1H), 7.67 (d, J=8.1 Hz, 1H), 5.11 (dd, J=13.3, 5.1 Hz, 1H), 4.48 (d, J=17.7 Hz, 1H), 4.35 (d, J=17.6 Hz, 1H), 2.99-2.82 (m, 1H), 2.66-2.55 (m, 1H), 2.40 (qd, J=13.3, 4.5 Hz, 1H), 2.01 (dtd, J=12.6, 5.3, 2.2 Hz, 1H).

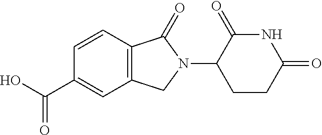 [SMILES: O=C1CCC(N2Cc3cc(C(=O)O)ccc3C2=O)C(=O)N1]

### CRBN Core 1: 2-(2,6-dioxo-3-piperidyl)-1-oxo-isoindoline-5-carboxylic Acid

To a stirred suspension of 3-(5-bromo-1-oxo-2,3-dihydro-1H-isoindol-2-yl)piperidine-2,6-dione (4.70 g, 13.8 mmol) in DMF (47 mL) at ambient temperature under nitrogen were successively added ethanedioic acid dihydrate (2.6 g, 20.7 mmol), diacetoxypalladium (310 mg, 1.38 mmol), (5-diphenylphosphanyl-9,9-dimethyl-xanthen-4-yl)-diphenyl-phosphane (800 mg, 1.38 mmol), N-ethyl-N-isopropyl-propan-2-amine (3.6 mL, 20.7 mmol) and acetic anhydride (1.9 mL, 20.7 mmol). The reaction mixture was stirred at 80° C. for 3 hours. The reaction mixture was triturated in water (100 ml), filtered and washed with DCM (50 mL) to afford 2-(2,6-dioxo-3-piperidyl)-1-oxo-isoindoline-5-carboxylic acid as a brown solid (3.8 g, 95% yield). ¹H NMR (400 MHz, DMSO-d6) δ 13.74 (s, 1H), 11.02 (s, 1H), 8.01 (s, 1H), 7.84 (s, 1H), 5.14 (dd, J=13.3, 5.1 Hz, 1H), 4.52 (d, J=17.9 Hz, 1H), 4.39 (d, J=17.8 Hz, 1H), 3.03-2.83 (m, 1H), 2.66-2.55 (m, 1H), 2.39 (td, J=13.2, 4.5 Hz, 1H), 2.03 (ddd, J=10.7, 5.4, 2.8 Hz, 1H).

### Example 1b: CRBN Core 2 Synthesis

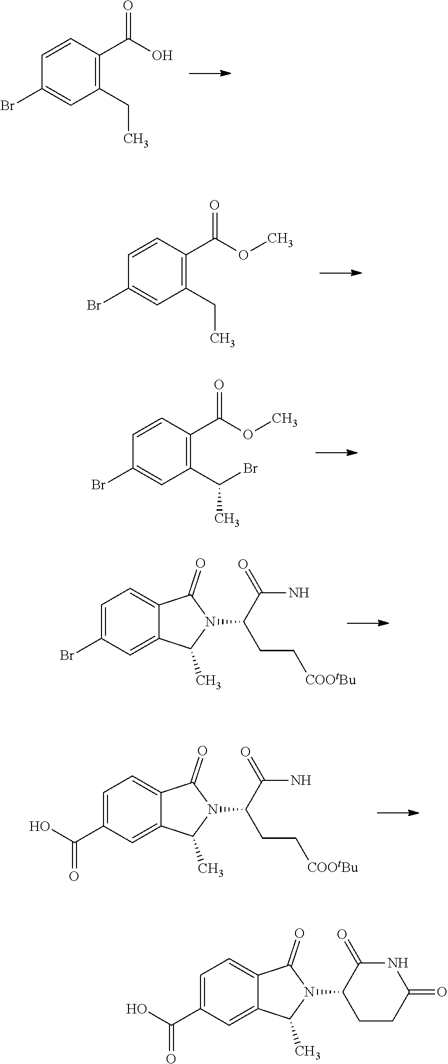

### CRBN Core 2

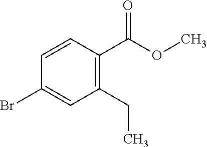

### methyl 4-bromo-2-ethyl-benzoate

To a solution of 4-bromo-2-ethylbenzoic acid (9.50 g, 39.4 mmol) in methanol (190 mL) was added sulfuric acid (4.2 mL, 78.8 mmol). The reaction was stirred at 65° C. for 16 hours and the solvent was evaporated under reduced pressure. The crude residue was dissolved in EtOAc (150 mL) and the layers were separated. The organic layer was washed with saturated NaHCO₃ solution (2×100 mL) and saturated NaCl (50 mL). The organic layer was dried through a layer separator and concentrated under reduced pressure to afford methyl 4-bromo-2-ethyl-benzoate (9.6 g, 98.2% yield) as a colorless oil. H NMR (400 MHz, DMSO-d6) δ 7.56-7.46 (m, 2H), 5.34 (t, J=5.5 Hz, 1H), 4.60 (dd, J=5.6, 2.6 Hz, 2H).

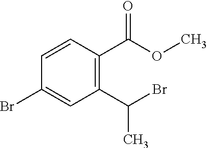

### methyl 4-bromo-2-(1-bromoethyl)benzoate

To a solution of methyl 4-bromo-2-ethyl-benzoate (9.6 g, 37.5 mmol) in DCE (144 mL) were added 2-[({E})-(1-cyano-1-methyl-ethyl)azo]-2-methyl-propanenitrile (154 mg, 0.938 mmol) and 1-bromopyrrolidine-2,5-dione (7.3 g, 41.3 mmol). The reaction mixture was stirred at 80° C. for 4 hours then cooled down to ambient temperature and diluted with EtOAc (150 mL). The layers were separated and the organic layer was washed with saturated NaHCO₃ solution (2×200 mL) and saturated NaCl solution (1×100 mL). The organic layer was dried over MgSO₄ and the filtrate was evaporated under reduced pressure to afford methyl 4-bromo-2-(1-bromoethyl)benzoate (12.4 g, 93.4% yield) as a yellow oil. ¹H NMR (400 MHz, DMSO-d6) δ 8.01 (d, J=2.0 Hz, 1H), 7.73 (d, J=8.4 Hz, 1H), 7.67 (dd, J=8.4, 1.9 Hz, 1H), 6.18 (q, J=6.9 Hz, 1H), 3.87 (s, 3H), 2.02 (d, J=6.9 Hz, 3H).

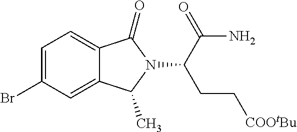

### tert-butyl (4S)-5-amino-4-[(3R)-5-bromo-3-methyl-1-oxo-isoindolin-2-yl]-5-oxo-pentanoate

To a solution of methyl 4-bromo-2-(1-bromoethyl)benzoate (11.3 g, 31.9 mmol) in acetonitrile (110 mL) were added tert-butyl (4S)-4-amino-4-carbamoylbutanoate hydrochloride (1.0 g, 41.5 mmol), dipotassium carbonate (11.0 g, 79.8 mmol) and sodium iodide (7.2 g, 47.9 mmol). The mixture was stirred at 75° C. for 16 hours. The crude residue was filtered and the filtrate was evaporated under reduced pressure. Water (200 mL) and EtOAc (200 mL) were added and the layers were separated. The aqueous layer was extracted with EtOAc (200 mL) and the combined organic layers were washed with saturated NaCl solution (200 mL), dried over MgSO₄ and evaporated under reduced pressure. The crude residue was purified by flash chromatography over silica gel (DCM/EtOAc/EtOH). The two diastereomers were separated by chiral SFC (Instrument: Waters Prep SFC200. Stationary Phase: Chiralpak IC 5 μm, 250×30 mm. Mobile phase: CO₂/(EtOH+0.5% IPAm) 80/20. Flow rate: 100 mL/min. UV detection: 254 nm. Temperature: 40° C. Pressure: 100 bars. Sample was dissolved by sonication in a mixture of 60 mL of (EtOH+0.5% IPAm), 100 mL of ACN, 100 mL MeOH and 100 mL of EtOH to a concentration of 27.47 mg/mL. This solution was heated and filtered on GHP 0.45 μm filter. Collection conditions: UV threshold:—C1 start 200 mAU; stop 80 mAU—C2 start 250 mAU; stop 30 mAU. Stacking conditions: Interval: 16 min. Elution time: 40 min. Injections: injections of 4.5 mL (123.61 mg) to afford:

tert-butyl (4S)-5-amino-4-[(3S)-5-bromo-3-methyl-1-oxo-isoindolin-2-yl]-5-oxo-pentanoate (4.09 g, 31.1% yield); ¹H NMR (400 MHz, DMSO-d6): δ (ppm) 7.92 (d, J=1.7 Hz, 1H), 7.69 (dd, J=8.0, 1.7 Hz, 1H), 7.61 (d, J=8.0 Hz, 1H), 7.45 (s, 1H), 7.21 (s, 1H), 4.71 (d, J=6.7 Hz, 1H), 4.47 (dd, J=9.3, 5.5 Hz, 1H), 2.32-1.97 (m, 3H), 1.50 (d, J=6.7 Hz, 3H), 1.38 (s, 9H); and

tert-butyl (4S)-5-amino-4-[(3R)-5-bromo-3-methyl-1-oxo-isoindolin-2-yl]-5-oxo-pentanoate (4.64 g, 35.3% yield); ¹H NMR (400 MHz, DMSO-d6): δ (ppm) 7.90 (d, J=1.7 Hz, 1H), 7.69 (dd, J=8.0, 1.7 Hz, 1H), 7.61 (d, J=8.0 Hz, 1H), 7.20 (s, 1H), 7.14 (s, 1H), 4.66 (q, J=6.7 Hz, 1H), 4.52-4.35 (m, 1H), 2.39-2.23 (m, 1H), 2.24-2.04 (m, 3H), 1.42 (d, J=6.7 Hz, 3H), 1.36 (s, 9H).

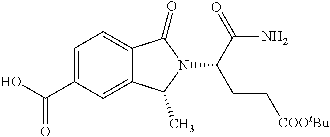

### (3R)-2-[(1S)-4-tert-butoxy-1-carbamoyl-4-oxo-butyl]-3-methyl-1-oxo-isoindoline-5-carboxylic Acid

To a solution of tert-butyl (4S)-5-amino-4-[(3R)-5-bromo-3-methyl-1-oxo-isoindolin-2-yl]-5-oxo-pentanoate (3.0 g, 7.17 mmol) in butyronitrile (24 mL) were added ethanedioic acid dihydrate (1.4 g, 10.8 mmol), diacetoxypalladium (50 mg, 0.215 mmol) and (5-diphenylphosphanyl-9,9-dimethyl-xanthen-4-yl)-diphenyl-phosphane (125 mg, 0.215 mmol). The reaction mixture was degassed with nitrogen then acetic anhydride (0.99 mL, 10.8 mmol) and N-ethyl-N-isopropyl-propan-2-amine (1.9 mL, 10.8 mmol) were added. The mixture was stirred at 75° C. for 2.5 hours then MeTHF (30 mL) and water (30 mL) were added. The layers were separated and the aqueous layer was extracted with MeTHF (25 mL). Combined organic layers were dried through a layer separator and evaporated under reduced pressure to afford (3R)-2-[(1S)-4-tert-butoxy-1-carbamoyl-4-oxo-butyl]-3-methyl-1-oxo-isoindoline-5-carboxylic acid (3.2 g, 94.5% yield) as an orange solid. 1H NMR (400 MHz, DMSO-d6): δ (ppm) 13.32 (s, 1H), 8.15 (s, 1H), 8.06 (dd, J=8.0, 1.4 Hz, 1H), 7.78 (d, J=7.9 Hz, 1H), 7.24 (s, 1H), 7.18 (s, 1H), 4.73 (q, J=6.7 Hz, 1H), 4.49-4.41 (m, 1H), 2.33 (ddd, J=12.4, 8.0, 5.3 Hz, 1H), 2.24-2.09 (m, 4H), 1.45 (d, J=6.8 Hz, 3H), 1.36 (s, 11H).

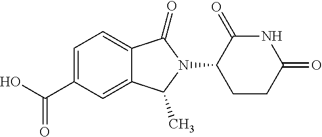 [SMILES: C[C@@H]1c2cc(C(=O)O)ccc2C(=O)N1[C@H]1CCC(=O)NC1=O]

### CRBN Core 2: (3R)-2-[(3S)-2,6-dioxo-3-piperidyl]-3-methyl-1-oxo-isoindoline-5-carboxylic Acid

To a solution of (3R)-2-[(1S)-4-tert-butoxy-1-carbamoyl-4-oxo-butyl]-3-methyl-1-oxo-isoindoline-5-carboxylic acid (200 mg, 0.425 mmol) in acetonitrile (2.1 mL) at ambient temperature under nitrogen was added 4-methylbenzenesulfonic acid hydrate (111 mg, 0.584 mmol). The reaction mixture was stirred at 85° C. for 2 hours then the suspension was filtered, washed with MeCN (2×2 mL) and dried under reduced pressure to afford as a white solid (3R)-2-[(3S)-2,6-dioxo-3-piperidyl]-3-methyl-1-oxo-isoindoline-5-carboxylic acid (113 mg, 87.1% yield). ¹H NMR (400 MHz, DMSO-d6) δ 13.36 (s, 1H), 10.99 (s, 1H), 8.19 (s, 1H), 8.07 (dd, J=8.0, 1.4 Hz, 1H), 7.79 (d, J=7.9 Hz, 1H), 2.91-2.78 (m, 1H), 2.71-2.55 (m, 2H), 2.01 (dt, J=9.8, 5.4 Hz, 1H), 1.46 (d, J=6.7 Hz, 3H).

### Example 1C: CRBN Core 3 Synthesis

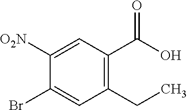

### 4-bromo-2-ethyl-5-nitro-benzoic Acid

To a solution of 4-bromo-2-ethylbenzoic acid (500 mg, 2.07 mmol) in sulfuric acid (5.0 mL, 93.8 mmol) at 0° C. under nitrogen was added nitric acid (0.10 mL, 2.28 mmol). The reaction mixture was stirred from 0° C. to ambient temperature for 2 hours. The reaction mixture was poured into iced water (50 mL), the suspension was filtered, washed with water (2×5 mL) and dried under reduced pressure to afford as a white solid, 4-bromo-2-ethyl-5-nitro-benzoic acid (570 mg, 99.3% yield). ¹H NMR (400 MHz, DMSO-d6) δ 8.36 (s, 1H), 7.94 (s, 1H), 3.00 (q, J=7.4 Hz, 2H), 1.18 (t, J=7.4 Hz, 3H).

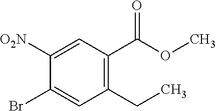 [SMILES: CCc1cc(Br)c([N+](=O)[O-])cc1C(=O)OC]

### methyl 4-bromo-2-ethyl-5-nitro-benzoate

To a solution of 4-bromo-2-ethyl-5-nitro-benzoic acid (570 mg, 2.06 mmol) in methanol (10.3 mL) at ambient temperature under nitrogen was added sulfuric acid (0.22 mL, 4.12 mmol). The reaction mixture was stirred at reflux for 16 hours then concentrated under reduced pressure. The crude residue was purified by flash chromatography over silica gel column (CyHex/EtOAc) to afford methyl 4-bromo-2-ethyl-5-nitro-benzoate (590 mg, 98.5% yield) as a colorless oil. ¹H NMR (400 MHz, DMSO-d6) δ 8.38 (s, 1H), 7.98 (s, 1H), 3.88 (s, 3H), 2.97 (q, J=7.5 Hz, 2H), 1.16 (dt, J=13.4, 7.5 Hz, 3H).

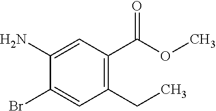

### methyl 5-amino-4-bromo-2-ethyl-benzoate

To a stirred suspension of methyl 4-bromo-2-ethyl-5-nitro-benzoate (590 mg, 2.05 mmol) in methanol (10 mL) at ambient temperature under nitrogen were added iron powder (139 mg, 2.46 mmol) and acetic acid (0.23 mL, 4.10 mmol). The reaction mixture was stirred at 40° C. for 2 hours. The reaction mixture was concentrated under reduced pressure. The crude residue was purified by flash chromatography over silica gel column (CyHex/EtOAc) to afford methyl 5-amino-4-bromo-2-ethyl-benzoate (300 mg, 56.8 yield) as a colorless oil. ¹H NMR (400 MHz, DMSO-d6) δ 7.32 (s, 1H), 7.24 (s, 1H), 5.39 (s, 2H), 3.79 (s, 3H), 2.71 (q, J=7.4 Hz, 2H), 1.07 (t, J=7.4 Hz, 3H).

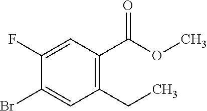

### methyl 4-bromo-2-ethyl-5-fluoro-benzoate

To a stirred suspension of methyl 5-amino-4-bromo-2-ethyl-benzoate (1.1 g, 4.26 mmol) in hydrogen tetrafluoroborate (48% in water, 5.6 mL, 42.6 mmol) at 0° C. under nitrogen was added sodium nitrite (441 mg, 6.39 mmol) in water (1 mL). The reaction mixture was stirred at 0° C. for 1 hour. The suspension was filtered and dried under reduced pressure. The solid was triturated in MeCN/Et2O (1/9), filtered and dried under reduced pressure to afford 2-bromo-4-ethyl-5-methoxycarbonyl-benzenediazonium tetrafluoroborate (900 mg, 59.2% yield) as a white powder. ¹H NMR (400 MHz, DMSO-d6) δ 9.31 (s, 1H), 8.40 (s, 1H), 3.94 (s, 3H), 3.11 (q, J=7.4 Hz, 2H), 1.23 (t, J=7.5 Hz, 3H).

2-Bromo-4-ethyl-5-methoxycarbonyl-benzenediazonium tetrafluoroborate (900 mg) was heated neat at 140° C. for 2 hours. The residue was diluted in EtOAc (50 mL) and the organic layer was washed with water, dried over sodium sulfate, filtered and concentrated under reduced pressure. The crude residue was purified by flash chromatography over silica gel column (Hept/EtOAc) to afford methyl 4-bromo-2-ethyl-5-fluoro-benzoate (290 mg, 26.1% yield) as a yellow oil. ¹H NMR (400 MHz, DMSO-d6) δ 7.75 (d, J=7.0 Hz, 1H), 7.68 (d, J=9.4 Hz, 1H), 3.85 (s, 3H), 2.87 (q, J=7.3 Hz, 2H), 1.14 (t, J=7.5 Hz, 3H).

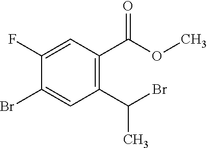

### methyl 4-bromo-2-(1-bromoethyl)-5-fluoro-benzoate

To a solution of methyl 4-bromo-2-ethyl-5-fluoro-benzoate (290 mg, 1.11 mmol) in acetonitrile (2 mL) at ambient temperature under nitrogen was added 1-bromopyrrolidine-2,5-dione (217 mg, 1.22 mmol). The reaction mixture was stirred under LED visible light at 75° C. for 1 hour then saturated Na₂S₂O₃ solution (10 mL) and EtOAc (10 mL) were added. The layers were separated and the aqueous layer was extracted with EtOAc (3×10 mL). The combined organic layers were dried over sodium sulfate, filtered and concentrated under reduced pressure. The crude residue was purified by flash chromatography over silica gel column (Hept/EtOAc) to afford methyl 4-bromo-2-(1-bromoethyl)-5-fluoro-benzoate (290 mg, 76.7% yield) as a colorless oil. ¹H NMR (400 MHz, DMSO-d6) δ 8.17 (d, J=6.8 Hz, 1H), 7.73 (d, J=9.2 Hz, 1H), 6.15 (q, J=7.0 Hz, 1H), 3.88 (s, 3H), 2.04-1.99 (m, 3H).

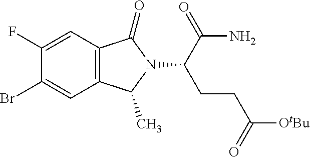 [SMILES: C[C@@H]1c2cc(Br)c(F)cc2C(=O)N1[C@@H](CCC(=O)OC(C)(C)C)C(N)=O]

### tert-butyl (4S)-5-amino-4-[(1S)-6-bromo-5-fluoro-1-methyl-3-oxo-isoindolin-2-yl]-5-oxo-pentanoate

To a solution of methyl 4-bromo-2-(1-bromoethyl)-5-fluoro-benzoate (290 mg, 0.853 mmol) in acetonitrile (3 mL) at ambient temperature under nitrogen were successively added sodium iodide (192 mg, 1.28 mmol), dipotassium carbonate (295 mg, 2.13 mmol) and tert-butyl (4S)-4-amino-4-carbamoylbutanoate hydrochloride (231 mg, 0.938 mmol). The reaction mixture was stirred at ambient temperature for 6 hours then saturated NH₄Cl solution (25 mL) and EtOAc (25 mL) were added. The layers were separated and the aqueous layer was extracted with EtOAc (3×10 mL). The combined organic layers were dried over sodium sulfate, filtered and concentrated under reduced pressure. The crude residue was purified by flash chromatography over silica gel column (CyHex/EtOAc) to afford tert-butyl (4S)-5-amino-4-[(1SR)-6-bromo-5-fluoro-1-methyl-3-oxo-isoindolin-2-yl]-5-oxo-pentanoate (260 mg, 71.0% yield) as a yellow foam. The two diastereomers were separated by chiral SFC (Instrument: Waters Prep SFC80. Stationary Phase: Chiralpak IC 5 μm, 250×20 mm. Mobile phase: CO2/(EtOH+0.5% IPAm) 80/20. Flow rate: 50 mL/min. UV detection: 220 nm. Temperature: 40° C. Pressure: 100 bars. Sample: batch was dissolved by sonication in 10 mL of ethanol to a concentration of 14 mg/mL. This solution was filtered on GHP 0.45 μm filter. Collection conditions: UV threshold:—C1 start 25 mAU; stop 25 mAU—C2 start 25 mAU; stop 25 mAU. Elution time: 20 min. Injections: 10 injections of 1 mL (14 mg) to afford:

(4S)-5-amino-4-[(1S)-6-bromo-5-fluoro-1-methyl-3-oxo-isoindolin-2-yl]-5-oxo-pentanoate (62 mg, 17% yield); ¹H NMR (400 MHz, DMSO-d6) δ 8.08 (d, J=6.0 Hz, 1H), 7.63 (d, J=7.6 Hz, 1H), 7.46 (s, 1H), 7.23 (s, 1H), 4.70 (q, J=6.6 Hz, 1H), 4.46 (dd, J=9.0, 5.2 Hz, 1H), 2.33-1.95 (m, 4H), 1.49 (d, J=6.7 Hz, 3H), 1.38 (s, 9H); and

(4S)-5-amino-4-[(1R)-6-bromo-5-fluoro-1-methyl-3-oxo-isoindolin-2-yl]-5-oxo-pentanoate (59.6 mg, 16% yield); ¹H NMR (400 MHz, DMSO-d6) δ 8.07 (d, J=6.0 Hz, 1H), 7.63 (d, J=7.6 Hz, 1H), 7.22 (s, 1H), 7.16 (s, 1H), 4.65 (q, J=6.7 Hz, 1H), 4.47-4.35 (m, 1H), 2.41-2.02 (m, 4H), 1.42 (d, J=6.8 Hz, 3H), 1.37 (s, 9H).

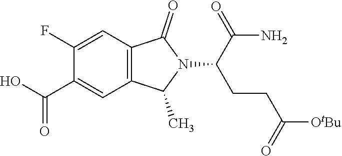 [SMILES: C[C@@H]1c2cc(C(=O)O)c(F)cc2C(=O)N1[C@@H](CCC(=O)OC(C)(C)C)C(N)=O]

### (3R)-2-[(1S)-4-tert-butoxy-1-carbamoyl-4-oxo-butyl]-6-fluoro-3-methyl-1-oxo-isoindoline-5-carboxylic Acid

To a solution of tert-butyl (4S)-5-amino-4-[(1R)-6-bromo-5-fluoro-1-methyl-3-oxo-isoindolin-2-yl]-5-oxo-pentanoate (47 mg, 0.109 mmol) in DMF (0.5 mL) were added (5-diphenylphosphanyl-9,9-dimethyl-xanthen-4-yl)-diphenyl-phosphane (6.3 mg, 0.011 mmol), diacetoxypalladium (2.5 mg, 0.011 mmol), ethanedioic acid dihydrate (41 mg, 0.328 mmol), N-ethyl-N-isopropyl-propan-2-amine (0.029 mL, 0.164 mmol) and acetic anhydride (0.015 mL, 0.164 mmol). The reaction mixture was stirred under nitrogen at 85° C. for 16 hours then the solvent was evaporated under reduced pressure and the crude residue was purified by flash chromatography over silica gel column (DCM/MeOH) to afford (3R)-2-[(1S)-4-tert-butoxy-1-carbamoyl-4-oxo-butyl]-6-fluoro-3-methyl-1-oxo-isoindoline-5-carboxylic acid (27 mg, 62.5% yield). Due to low amount of material obtained, the product was engaged in the next step without NMR analysis. LCMS (ESI) m/z 417 [M+Na]⁺.

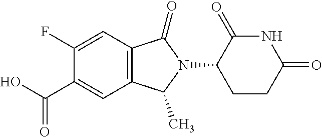 [SMILES: C[C@@H]1c2cc(C(=O)O)c(F)cc2C(=O)N1[C@H]1CCC(=O)NC1=O]

### CRBN core 3: (3R)-2-[(3S)-2,6-dioxo-3-piperidyl]-6-fluoro-3-methyl-1-oxo-isoindoline-5-carboxylic Acid

To a solution of (3R)-2-[(1S)-4-tert-butoxy-1-carbamoyl-4-oxo-butyl]-6-fluoro-3-methyl-1-oxo-isoindoline-5-carboxylic acid (27 mg, 0.068 mmol) in acetonitrile (0.3 mL) was added 4-methylbenzenesulfonic acid hydrate (18 mg, 0.094 mmol). The reaction mixture was stirred at 85° C. for 3 hours then filtered. The resulting solid was washed twice with ACN to afford (3R)-2-[(3S)-2,6-dioxo-3-piperidyl]-6-fluoro-3-methyl-1-oxo-isoindoline-5-carboxylic acid (11 mg, 50.2% yield) as a white solid. Due to low amount of material obtained, the product was engaged in the next step without NMR analysis. LCMS (ESI) m/z 321 [M+H]⁺.

### Example 1D: CRBN Core 4 Synthesis

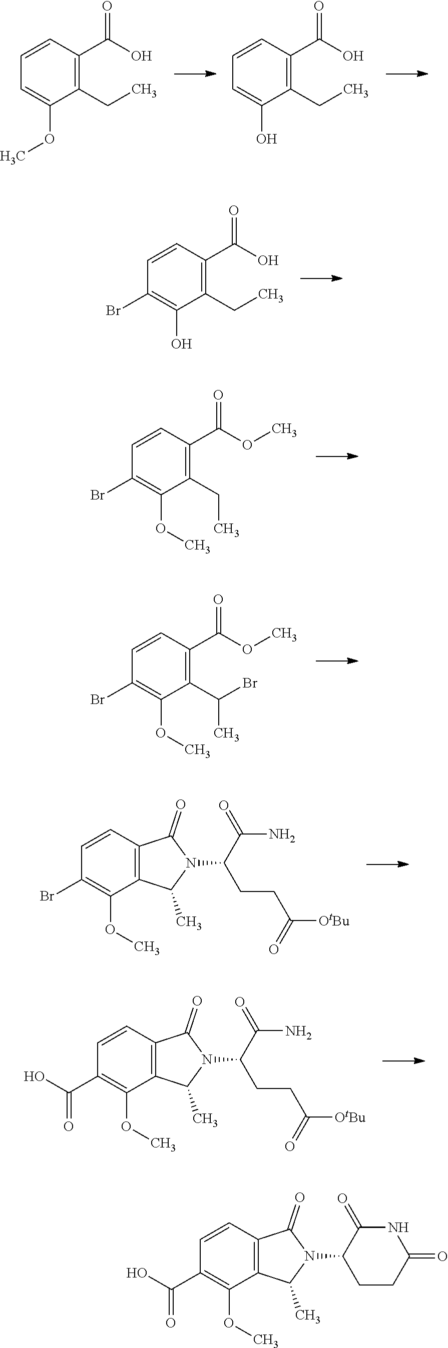

### CRBN Core 4

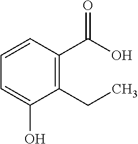

### 2-ethyl-3-hydroxy-benzoic Acid

To a solution of 2-ethyl-3-methoxybenzoic acid (98%, 5.0 g, 27.2 mmol) in DCM (68 mL) at −78° C. under nitrogen was added tribromoborane (1M in DCM, 41 mL, 40.8 mmol). The reaction mixture was stirred at ambient temperature for 16 hours then MeOH was carefully added and the solvent was evaporated under reduced pressure to afford 2-ethyl-3-hydroxy-benzoic acid (4.5 g, 99% yield) as an orange solid. ¹H NMR (400 MHz, DMSO-d6): δ (ppm) 12.68 (s, 1H), 9.60 (d, J=41.7 Hz, 1H), 7.13 (dt, J=7.7, 1.6 Hz, 1H), 7.09-7.02 (m, 1H), 6.96 (ddd, J=14.8, 7.9, 1.5 Hz, 1H), 2.79 (dq, J=18.0, 7.3 Hz, 2H), 1.19-0.96 (m, 3H).

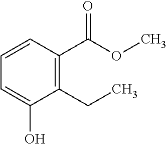

### methyl 2-ethyl-3-hydroxy-benzoate

To a solution of 2-ethyl-3-hydroxy-benzoic acid (4.5 g, 27.1 mmol) in methanol (90 mL) was added thionyl chloride (3.2 mL, 43.3 mmol). The reaction mixture was stirred at 85° C. for 3 hours then the solvent was evaporated under reduced pressure. The crude residue was purified by flash chromatography over silica gel column (DCM/MeOH) to afford methyl 2-ethyl-3-hydroxy-benzoate (3.0 g, 62.1% yield) as a white solid. ¹H NMR (400 MHz, DMSO-d6) δ 9.63 (s, 1H), 7.13 (dd, J=7.7, 1.6 Hz, 1H), 7.08 (t, J=7.8 Hz, 1H), 6.99 (dd, J=7.9, 1.6 Hz, 1H), 3.80 (s, 3H), 2.78 (q, J=7.4 Hz, 2H), 1.08 (t, J=7.3 Hz, 3H).

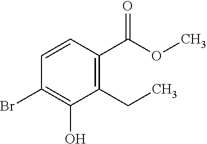

### methyl 4-bromo-2-ethyl-3-hydroxy-benzoate

To a solution of methyl 2-ethyl-3-hydroxy-benzoate (2.4 g, 13.6 mmol) in DCM (68 mL) was added N-isopropylpropan-2-amine (760 μL, 5.42 mmol). The reaction mixture was stirred at ambient temperature for 5 minutes then 1-bromopyrrolidine-2,5-dione (2.4 g, 13.6 mmol) was added. The reaction mixture was stirred at ambient temperature for 5 minutes then the solvent was evaporated under reduced pressure. The crude residue was purified by flash chromatography over silica gel column (Cyclohexane/EtOAc) to afford methyl 4-bromo-2-ethyl-3-hydroxy-benzoate (3.2 g, 90.8% yield) as a white solid. ¹H NMR (400 MHz, DMSO-d6) δ 9.28 (s, 1H), 7.46 (d, J=8.4 Hz, 1H), 7.15 (d, J=8.4 Hz, 1H), 3.81 (s, 3H), 2.88 (q, J=7.3 Hz, 2H), 1.14-1.05 (m, 3H).

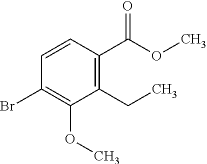

### methyl 4-bromo-2-ethyl-3-methoxy-benzoate

To a solution of methyl 4-bromo-2-ethyl-3-hydroxy-benzoate (1.6 g, 6.18 mmol) in acetone (30 mL) were added dipotassium carbonate (4.3 g, 30.9 mmol) and iodomethane (1.2 mL, 18.5 mmol). The reaction mixture was stirred at 50° C. for 1.5 hours then EtOAc was added. The reaction mixture was washed with saturated NH₄Cl solution then saturated NaCl solution. The organic layer was dried over Na₂SO₄, filtered and evaporated under reduced pressure to afford methyl 4-bromo-2-ethyl-3-methoxy-benzoate (1.65 g, 97.8% yield). ¹H NMR (400 MHz, DMSO-d6) δ 7.61 (d, J=8.4 Hz, 1H), 7.45 (d, J=8.4 Hz, 1H), 3.84 (s, 3H), 3.80 (s, 3H), 2.91 (q, J=7.4 Hz, 2H), 1.12 (t, J=7.4 Hz, 3H).

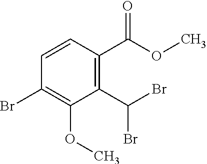 [SMILES: COC(=O)c1ccc(Br)c(OC)c1C(Br)Br]

### methyl 4-bromo-2-(1-bromoethyl)-3-methoxy-benzoate

To a solution of methyl 4-bromo-2-ethyl-3-methoxy-benzoate (761 mg, 2.79 mmol) in trifluorotoluene (14 mL) at ambient temperature under nitrogen were added 1-bromopyrrolidine-2,5-dione (601 mg, 3.34 mmol) and benzoyl benzenecarboperoxoate (202 mg, 0.836 mmol). The reaction mixture was stirred at 110° C. for 1 hour. The reaction mixture was quenched with saturated Na₂S₂O₃ solution and EtOAc was added. The layers were separated and the aqueous layer was extracted with EtOAc. The combined organic layers were dried over sodium sulfate, filtered and concentrated under reduced pressure. The crude residue was purified by flash chromatography over silica gel column (Cyclohexane/EtOAc) to afford methyl 4-bromo-2-(1-bromoethyl)-3-methoxy-benzoate (916 mg, 93.4% yield). ¹H NMR (400 MHz, DMSO-d6) δ 7.76 (d, J=8.3 Hz, 1H), 7.35 (d, J=8.4 Hz, 1H), 5.96 (q, J=7.1 Hz, 1H), 3.98 (s, 3H), 3.87 (s, 3H), 2.04 (d, J=7.0 Hz, 3H).

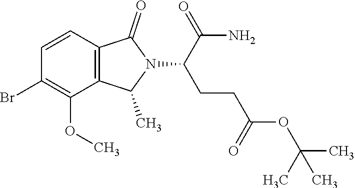 [SMILES: COc1c(Br)ccc2c1[C@@H](C)N([C@@H](CCC(=O)OC(C)(C)C)C(N)=O)C2=O]

### tert-butyl (4S)-5-amino-4-[(3SR)-5-bromo-4-methoxy-3-methyl-1-oxo-isoindolin-2-yl]-5-oxo-pentanoate

To a solution of methyl 4-bromo-2-(1-bromoethyl)-3-methoxy-benzoate (900 mg, 1.97 mmol) in acetonitrile (7 mL) at ambient temperature under nitrogen were successively added sodium iodide (443 mg, 2.95 mmol), dipotassium carbonate (680 mg, 4.92 mmol) and tert-butyl (4S)-4-amino-4-carbamoylbutanoate hydrochloride (484 mg, 1.97 mmol). The reaction mixture was stirred at 90° C. for 16 hours then saturated NH₄Cl solution (50 mL) and EtOAc (50 mL) were added. The layers were separated and the aqueous layer was extracted with EtOAc (3×25 mL). The combined organic layers were dried over sodium sulfate, filtered and concentrated under reduced pressure. The crude residue was purified by flash chromatography over silica gel column (CyHex/EtOAc/EtOH) then by reversed-phase flash chromatography (water/MeCN) to afford tert-butyl (4S)-5-amino-4-[(3SR)-5-bromo-4-methoxy-3-methyl-1-oxo-isoindolin-2-yl]-5-oxo-pentanoate (680 mg, 78% yield) as a white foam. This reaction was duplicated on 600 mg of methyl 4-bromo-2-(1-bromoethyl)-3-methoxy-benzoate to afford 420 mg, 72.5% yield of the same crude product. The two crude products were combined to afford 1.1 g of diastereomers mixture. The two diastereomers were separated by chiral SFC (Instrument: Waters Prep SFC200. Stationary Phase: Chiralpak IB 5 μm, 250×20 mm. Mobile phase: CO2/IpOH 80/20. Flow rate: 70 mL/min. UV detection: k=210 nm. Temperature: 40° C. Pressure: 100 bars. Sample: 1.07 g were dissolved by sonication in 20 mL of IpOH to a concentration of 53.5 mg/mL. This solution was filtered on GHP 0.45 μm filter. Collection conditions: UV threshold:—C1 start 1000 mAU; stop 500 mAU—C2 start 100 mAU; stop 500 mAU. Stacking conditions: interval: 2.05 min. Elution time: 4 min. Injections: 20 injections of 1 mL (53.5 mg) to afford:

tert-butyl (4S)-5-amino-4-[(3S)-5-bromo-4-methoxy-3-methyl-1-oxo-isoindolin-2-yl]-5-oxo-pentanoate (500 mg, 57.3% yield); ¹H NMR (400 MHz, DMSO-d6) δ 7.77 (d, J=8.0 Hz, 1H), 7.43 (s, 1H), 7.39 (d, J=8.0 Hz, 1H), 7.21 (s, 1H), 4.87 (q, J=6.6 Hz, 1H), 4.43-4.36 (m, 1H), 3.89 (s, 3H), 2.33-2.11 (m, 4H), 1.53 (d, J=6.7 Hz, 3H), 1.38 (s, 9H); and

tert-butyl (4S)-5-amino-4-[(3R)-5-bromo-4-methoxy-3-methyl-1-oxo-isoindolin-2-yl]-5-oxo-pentanoate (540 mg, 61.5% yield)¹H NMR (400 MHz, DMSO-d6) δ 7.76 (d, J=8.0 Hz, 1H), 7.38 (d, J=8.0 Hz, 1H), 7.17 (d, J=9.1 Hz, 2H), 4.86 (q, J=6.6 Hz, 1H), 4.49-4.18 (m, 1H), 3.90 (s, 3H), 2.43-2.04 (m, 4H), 1.46 (d, J=6.7 Hz, 3H), 1.37 (s, 9H).

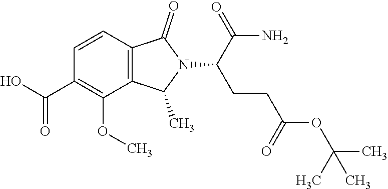 [SMILES: COc1c(C(=O)O)ccc2c1[C@@H](C)N([C@@H](CCC(=O)OC(C)(C)C)C(N)=O)C2=O]

### (3R)-2-[(1S)-4-tert-butoxy-1-carbamoyl-4-oxo-butyl]-4-methoxy-3-methyl-1-oxo-isoindoline-5-carboxylic Acid

A reaction vial was successively charged with tert-butyl (4S)-5-amino-4-[(3R)-5-bromo-4-methoxy-3-methyl-1-oxo-isoindolin-2-yl]-5-oxo-pentanoate (540 mg, 1.22 mmol), ethanedioic acid dihydrate (463 mg, 3.67 mmol), diacetoxypalladium (28 mg, 0.122 mmol) and (5-diphenylphosphanyl-9,9-dimethyl-xanthen-4-yl)-diphenyl-phosphane (72 mg, 0.122 mmol) in DMF (5 mL). The reaction mixture was degassed with nitrogen for 10 min then acetic anhydride (0.17 mL, 1.84 mmol) and N-ethyl-N-isopropyl-propan-2-amine (0.32 mL, 1.84 mmol) were added. The reaction mixture was stirred at 100° C. for 2 hours. The reaction mixture was then filtered through a pad of Celite and the filtrate was concentrated under reduced pressure. The crude residue was purified by flash chromatography over silica gel column (DCM/MeOH) to afford (3R)-2-[(1S)-4-tert-butoxy-1-carbamoyl-4-oxo-butyl]-4-methoxy-3-methyl-1-oxo-isoindoline-5-carboxylic acid (500 mg, 80.4% yield) as a white solid. ¹H NMR (400 MHz, DMSO-d6) δ 7.78 (d, J=7.8 Hz, 1H), 7.47 (d, J=7.8 Hz, 1H), 7.17 (d, J=14.5 Hz, 2H), 4.83 (q, J=6.6 Hz, 1H), 4.49-4.30 (m, 1H), 3.88 (s, 3H), 2.27-2.11 (m, 4H), 1.46 (d, J=6.6 Hz, 3H), 1.37 (s, 9H).

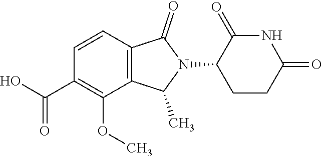 [SMILES: COc1c(C(=O)O)ccc2c1[C@@H](C)N([C@H]1CCC(=O)NC1=O)C2=O]

### CRBN core 4: (3R)-2-[(3S)-2,6-dioxo-3-piperidyl]-4-methoxy-3-methyl-1-oxo-isoindoline-5-carboxylic Acid

A reaction vial was successively charged with (3R)-2-[(1S)-4-tert-butoxy-1-carbamoyl-4-oxo-butyl]-4-methoxy-3-methyl-1-oxo-isoindoline-5-carboxylic acid (500 mg, 1.23 mmol) and 4-methylbenzenesulfonic acid hydrate (468 mg, 2.46 mmol) in acetonitrile (6 mL). The reaction mixture was stirred at 85° C. for 2 hours then concentrated under reduced pressure. The crude residue was purified by flash chromatography over silica gel column (DCM/MeOH) to afford (3R)-2-[(3S)-2,6-dioxo-3-piperidyl]-4-methoxy-3-methyl-1-oxo-isoindoline-5-carboxylic acid (200 mg, 48.9% yield) as a white foam. ¹H NMR (400 MHz, DMSO-d6) δ 13.29 (s, 1H), 10.98 (s, 1H), 7.81 (d, J=7.7 Hz, 1H), 7.48 (d, J=7.8 Hz, 1H), 4.93-4.75 (m, 2H), 3.89 (s, 3H), 2.94-2.78 (m, 1H), 2.71-2.55 (m, 2H), 2.06-2.01 (m, 1H), 1.47 (d, J=6.7 Hz, 3H).

### Example 1E: CRBN Core 5 Synthesis

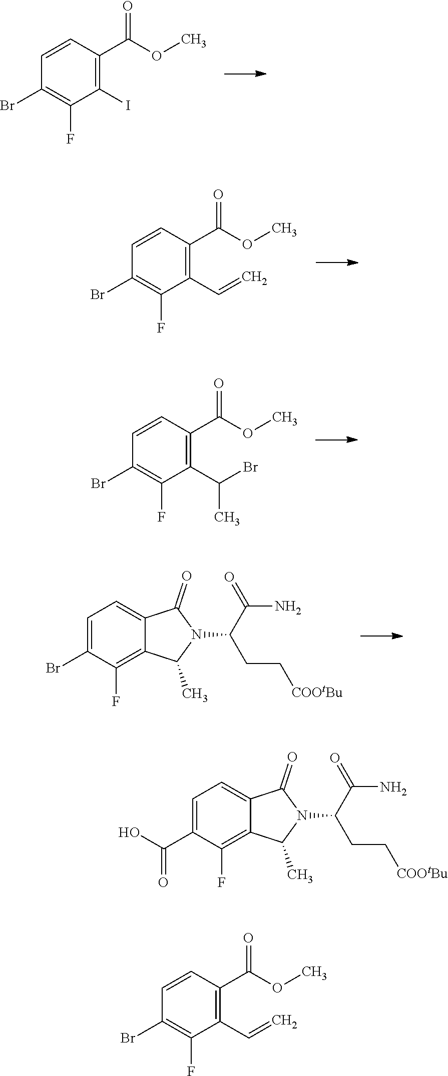

### methyl 4-bromo-3-fluoro-2-vinyl-benzoate

To a solution of methyl 4-bromo-3-fluoro-2-iodobenzoate (4.5 g, 12.5 mmol) in acetonitrile (55 mL) at ambient temperature under nitrogen were successively added 1,10-phenanthroline (113 mg, 0.627 mmol), diacetoxypalladium (141 mg, 0.627 mmol), tripotassium phosphate (7.9 g, 37.6 mmol) and 4,4,5,5-tetramethyl-2-vinyl-1,3,2-dioxaborolane (6.03 g, 37.6 mmol). The reaction mixture was stirred at 85° C. for 2 hours then the suspension was filtered and the solid was washed with EtOAc (100 mL). The filtrate was washed with saturated NH₄Cl solution (50 mL), dried over sodium sulfate, filtered and concentrated under reduced pressure. The crude residue was purified by flash chromatography over silica gel column (CyHex/EtOAc) to afford (methyl 4-bromo-3-fluoro-2-vinyl-benzoate (700 mg, 21.5% yield) as a colorless oil. ¹H NMR (400 MHz, DMSO-d6) δ 7.76 (dd, J=8.4, 6.6 Hz, 1H), 7.55 (dd, J=8.4, 1.3 Hz, 1H), 6.98-6.82 (m, 1H), 5.75-5.63 (m, 2H), 3.83 (s, 3H).

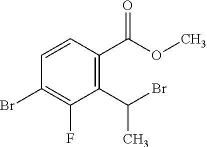

### methyl 4-bromo-2-(1-bromoethyl)-3-fluoro-benzoate

A solution of methyl 4-bromo-3-fluoro-2-vinyl-benzoate (450 mg, 1.74 mmol) in hydrogen bromide (33% in AcOH, 9.4 mL, 52.1 mmol) was stirred at ambient temperature for 48 hours. The reaction mixture was diluted in EtOAc (25 mL) and water (25 mL) then NaHCO₃ was added. The layers were separated and the aqueous layer was extracted with EtOAc (2×50 mL). The combined organic layers were dried over sodium sulfate, filtered and concentrated under reduced pressure. The crude residue was purified by flash chromatography over silica gel column (CyHex/EtOAc) to afford methyl 4-bromo-2-(1-bromoethyl)-3-fluoro-benzoate (330 mg, 55.9% yield) as a colorless oil. ¹H NMR (400 MHz, DMSO-d6) δ 7.86 (dd, J=8.5, 6.7 Hz, 1H), 7.53 (dd, J=8.5, 1.3 Hz, 1H), 6.08 (qd, J=7.0, 2.2 Hz, 1H), 3.89 (s, 3H), 2.02 (dd, J=7.0, 1.8 Hz, 3H).

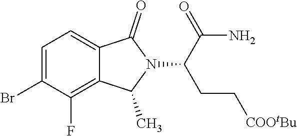

### tert-butyl (4S)-5-amino-4-(5-bromo-4-fluoro-3-methyl-1-oxo-isoindolin-2-yl)-5-oxo-pentanoate

To a solution of methyl 4-bromo-2-(1-bromoethyl)-3-fluoro-benzoate (330 mg, 0.971 mmol) in acetonitrile (4 mL) at ambient temperature under nitrogen were successively added tert-butyl (4S)-4-amino-4-carbamoylbutanoate hydrochloride (478 mg, 1.94 mmol), sodium iodide (218 mg, 1.46 mmol) and dipotassium carbonate (671 mg, 4.85 mmol). The reaction mixture was stirred at reflux for 1 hour then poured into a saturated NH₄Cl solution (20 mL) and EtOAc (20 mL). The layers were separated and the aqueous layer was extracted with EtOAc (2×20 mL). The combined organic layers were dried over sodium sulfate, filtered and concentrated under reduced pressure. The crude residue was purified by flash chromatography over silica gel column (CyHex/EtOAc/EtOH). The two diastereomers were separated by chiral SFC. Instrument: Waters Prep SFC200. Stationary Phase: Chiralpak IB 5 μm, 250×20 mm. Mobile layer: CO2/IpOH 90/10. Flow rate: 50 mL/minutes. UV detection: k=254 nm. Temperature: 40° C. Pressure: 100 bars. Sample: 698 mg were dissolved by sonication in 20 mL of IpOH to a concentration of 35 mg/mL. This solution was filtered on GHP 0.45 μm filter. Collection conditions: UV. Threshold:—C1 start 40 mAU; stop 120 mAU—C2 start 180 mAU; stop 20 mAU. Stacking conditions: interval: 12.75 minutes. Elution time: 17 minutes. Injections: 40 injections of 0.5 mL (17.5 mg) sample solution were realized for a global amount of m=698 mg to afford tert-butyl (4S)-5-amino-4-(5-bromo-4-fluoro-3-methyl-1-oxo-isoindolin-2-yl)-5-oxo-pentanoate (750 mg, 37% yield) as a yellow foam. ¹H NMR (400 MHz, DMSO) δ 7.85 (dd, J=8.0, 5.9 Hz, 1H), 7.48 (d, J=8.0 Hz, 1H), 7.19 (d, J=17.8 Hz, 2H), 4.89 (q, J=6.7 Hz, 1H), 4.41 (dd, J=9.0, 5.3 Hz, 1H), 2.41-2.02 (m, 4H), 1.46 (d, J=6.7 Hz, 3H), 1.36 (s, 9H).

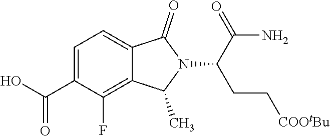

### (3R)-2-[(1S)-4-tert-butoxy-1-carbamoyl-4-oxo-butyl]-4-fluoro-3-methyl-1-oxo-isoindoline-5-carboxylic Acid

To a solution of tert-butyl (4S)-5-amino-4-[(3R)-5-bromo-4-fluoro-3-methyl-1-oxo-isoindolin-2-yl]-5-oxo-pentanoate (320 mg, 0.745 mmol) in butyronitrile (4 mL) at ambient temperature under nitrogen were successively added ethanedioic acid dihydrate (141 mg, 1.12 mmol), diacetoxypalladium (17 mg, 0.074 mmol), (5-diphenylphosphanyl-9,9-dimethyl-xanthen-4-yl)-diphenyl-phosphane (43 mg, 0.074 mmol), N-ethyl-N-isopropyl-propan-2-amine (0.20 mL, 1.12 mmol) and acetic anhydride (0.10 mL, 1.12 mmol). The reaction mixture was stirred at 80° C. for 2 hours then filtered and diluted with EtOAc (20 mL). The organic layer was washed with a 1M aqueous HCl solution (15 mL), dried over sodium sulfate, filtered and concentrated under reduced pressure. The crude residue was purified by flash chromatography over silica gel column (CyHex/EtOAc/EtOH) to afford (3R)-2-[(1S)-4-tert-butoxy-1-carbamoyl-4-oxo-butyl]-4-fluoro-3-methyl-1-oxo-isoindoline-5-carboxylic acid (150 mg, 51.0% yield) as a yellow foam. ¹H NMR (400 MHz, DMSO-d6) δ 13.57 (s, 1H), 7.97 (t, J=6.8 Hz, 1H), 7.59 (d, J=7.8 Hz, 1H), 7.20 (d, J=22.7 Hz, 2H), 4.89 (q, J=6.6 Hz, 1H), 4.43 (dd, J 8.9, 5.4 Hz, 1H), 2.45-2.04 (m, 4H), 1.48 (d, J=6.6 Hz, 3H), 1.36 (s, 9H).

### Example 1F: CRBN Core 6 Synthesis

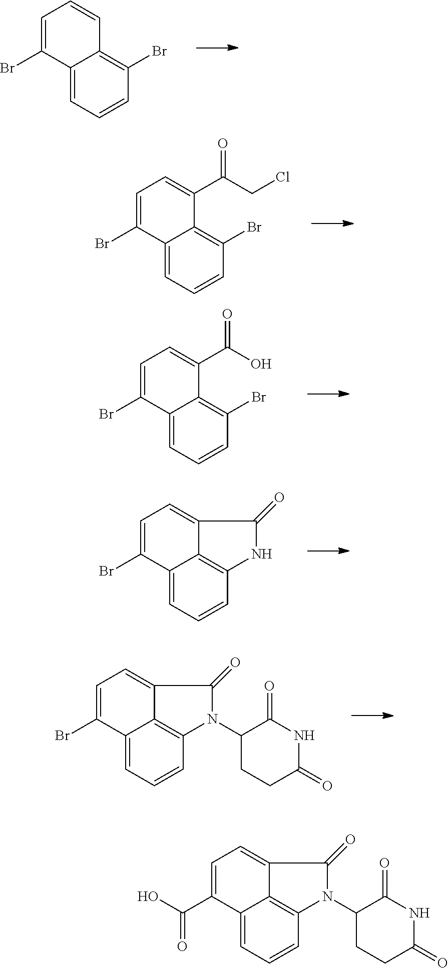

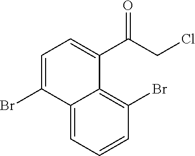 [SMILES: O=C(CCl)c1ccc(Br)c2cccc(Br)c12]

### 2-chloro-1-(4,8-dibromo-1-naphthyl)ethanone

To a solution of 1,5-dibromonaphthalene (5.0 g, 17.0 mmol) in DCE (60 mL) at 0° C. under nitrogen were added 2-chloroacetyl chloride (1.8 mL, 22.0 mmol) and aluminum trichloride (2.94 g, 22.0 mmol). The reaction mixture was allowed to warm up to ambient temperature and stirred for 16 hours. The reaction mixture was poured into ice (50 mL) and diluted with EtOAc (50 mL). The layers were separated and the aqueous layer was extracted with EtOAc (2×20 mL). The combined organic layers were dried over sodium sulfate, filtered and concentrated under reduced pressure to afford 2-chloro-1-(4,8-dibromo-1-naphthyl)ethanone (5.83 g, 94.8% yield) as a brown solid. H NMR (400 MHz, DMSO-d6) δ 8.37 (dd, J=8.6, 1.1 Hz, 1H), 8.11 (dd, J=7.5, 1.1 Hz, 1H), 8.09 (d, J=7.7 Hz, 1H), 7.69 (dd, J=8.5, 7.5 Hz, 1H), 7.59 (d, J=7.7 Hz, 1H), 5.05 (s, 2H).

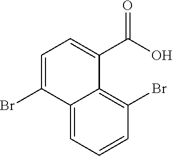 [SMILES: O=C(O)c1ccc(Br)c2cccc(Br)c12]

### 4,8-dibromonaphthalene-1-carboxylic Acid

To a solution of 2-chloro-1-(4,8-dibromo-1-naphthyl)ethanone (5.83 g, 16.1 mmol) in sulfuric acid (69.2 mL) at ambient temperature under nitrogen was added sodium nitrite (1.17 g, 16.9 mmol). The reaction mixture was stirred at 65° C. for 2 hours then poured into ice (50 mL) and EtOAc (50 mL) was added. The layers were separated and the aqueous layer was extracted with EtOAc (2×20 mL). NaOH 33% aqueous solution (250 mL) was added to the combined organic layers. The suspension was filtered and the filtrate was acidified with HCl 37% solution (75 mL) then filtered. The solid was dissolved in EtOAc (50 mL), washed with saturated NaCl solution (50 mL), dried over sodium sulfate, filtered and concentrated under reduced pressure to afford 4,8-dibromonaphthalene-1-carboxylic acid (3.98 g, 74.9% yield) as a brown solid. ¹H NMR (400 MHz, DMSO-d6) δ 8.33 (dd, J=8.5, 1.1 Hz, 1H), 8.09 (dd, J=7.5, 1.2 Hz, 1H), 8.02 (d, J=7.7 Hz, 1H), 7.68-7.62 (m, 1H), 7.59 (d, J=7.8 Hz, 1H).

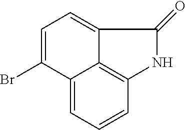 [SMILES: O=C1Nc2cccc3c(Br)ccc1c23]

### 5-bromo-1H-benzo[cd]indol-2-one

To a stirred suspension of 4,8-dibromonaphthalene-1-carboxylic acid (3.73 g, 11.3 mmol) in ammonium hydroxide (36 mL, 0.226 mol) was added copper (187 mg, 2.94 mmol). The reaction mixture was stirred at 80° C. for 2 hours then poured into ice-cooled water and acidified with concentrated HCl until pH 2. The resulting yellow precipitate was filtered off and dried under reduced pressure. The crude residue was purified by flash chromatography over silica gel column (DCM/MeOH) to afford 5-bromo-1H-benzo[cd]indol-2-one (900 mg, 32.1% yield) as an orange solid. ¹H NMR (400 MHz, DMSO-d6) δ 10.87 (s, 1H), 8.07 (dd, J=7.4, 1.3 Hz, 1H), 7.89 (dd, J=7.4, 1.3 Hz, 1H), 7.63 (ddd, J=8.3, 7.0, 1.3 Hz, 1H), 7.58-7.51 (m, 1H), 7.05 (dd, J=7.0, 1.3 Hz, 1H).

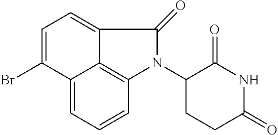 [SMILES: O=C1CCC(N2C(=O)c3ccc(Br)c4cccc2c34)C(=O)N1]

### 3-(5-bromo-2-oxo-benzo[cd]indol-1-yl)piperidine-2,6-dione

To a suspension of 5-bromo-1H-benzo[cd]indol-2-one (580 mg, 2.34 mmol) in THF (23 mL) maintained below 5° C. was added sodium hydride (60%, 935 mg, 23.4 mmol). The reaction mixture was allowed to warm up to ambient temperature and stirred for 15 minutes then cooled down to 0° C. 3-Bromopiperidine-2,6-dione (2.3 g, 11.7 mmol) was added and the reaction mixture was stirred at 60° C. for 2 hours. The reaction mixture was slowly poured into ice cold water and extracted with EtOAc. The combined organic layers were dried over Na₂SO₄ and concentrated under reduced pressure. The crude residue was purified by flash chromatography over silica gel column (CyHex/EtOAc/EtOH) to afford 3-(5-bromo-2-oxo-benzo[cd]indol-1-yl)piperidine-2,6-dione (304 mg, 36.2% yield) as an orange solid. ¹H NMR (400 MHz, DMSO-d6) δ 11.14 (s, 1H), 8.13 (d, J=7.6 Hz, 1H), 8.00 (d, J=7.4 Hz, 1H), 7.72-7.61 (m, 2H), 7.26 (d, J=6.9 Hz, 1H), 5.47 (dd, J=12.9, 5.4 Hz, 1H), 2.95 (ddd, J=17.2, 13.2, 5.3 Hz, 1H), 2.83-2.61 (m, 2H), 2.17-2.06 (m, 1H).

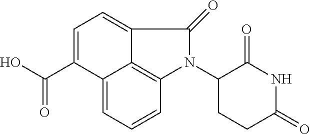 [SMILES: O=C1CCC(N2C(=O)c3ccc(C(=O)O)c4cccc2c34)C(=O)N1]

### CRBN core 6: 1-(2,6-dioxo-3-piperidyl)-2-oxo-benzo[cd]indole-5-carboxylic Acid

To a solution of 3-(5-bromo-2-oxo-benzo[cd]indol-1-yl)piperidine-2,6-dione (350 mg, 0.974 mmol) in butyronitrile (3 mL) were added ethanedioic acid dihydrate (184 mg, 1.46 mmol), diacetoxypalladium (23 mg, 0.097 mmol) and (5-diphenylphosphanyl-9,9-dimethyl-xanthen-4-yl)-diphenyl-phosphane (56 mg, 0.097 mmol). The reaction mixture was degassed by nitrogen bubbling then acetic anhydride (0.14 mL, 1.46 mmol) and N-ethyl-N-isopropyl-propan-2-amine (0.26 mL, 1.46 mmol) were added. The mixture was stirred at 80° C. for 3 hours then diluted with EtOAc and water. The layers were separated and the aqueous layer was further extracted with EtOAc (3×20 mL). The combined organic layers were washed with saturated NaCl solution, dried over sodium sulfate, filtered and concentrated under reduced pressure. The crude residue was purified by flash chromatography over silica gel column (DCM/MeOH, 1% acetic acid) to afford 1-(2,6-dioxo-3-piperidyl)-2-oxo-benzo[cd]indole-5-carboxylic acid (229 mg, 70.3% yield) as a yellow solid. ¹H NMR (400 MHz, DMSO-d6) δ 13.71 (s, 1H), 11.14 (s, 1H), 8.41 (dd, J=17.3, 8.0 Hz, 2H), 8.15 (d, J=7.3 Hz, 1H), 7.62 (dd, J=8.8, 7.2 Hz, 1H), 7.21 (d, J=7.2 Hz, 1H), 5.47 (dd, J=12.8, 5.4 Hz, 1H), 2.95 (ddd, J=16.8, 13.3, 5.2 Hz, 1H), 2.86-2.60 (m, 2H), 2.12 (ddd, J=12.6, 5.8, 3.6 Hz, 1H).

### Example 2: Coupling Procedure A: Isoindoline Coupling with CRBN Core 1

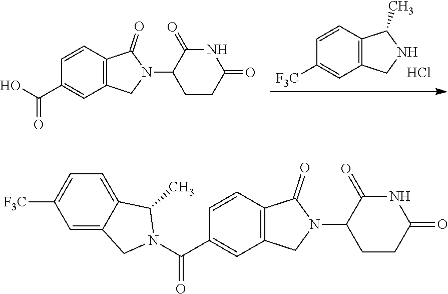 [SMILES: O=C1CCC(N2Cc3cc(C(=O)O)ccc3C2=O)C(=O)N1 ; C[C@@H]1NCc2cc(C(F)(F)F)ccc21 ; Cl ; C[C@H]1c2ccc(C(F)(F)F)cc2CN1C(=O)c1ccc2c(c1)CN(C1CCC(=O)NC1=O)C2=O]

### Representative Example of Coupling Procedure A

To a solution of 2-(2,6-dioxo-3-piperidyl)-1-oxo-isoindoline-5-carboxylic acid (60 mg, 0.208 mmol) and (1S)-1-methyl-5-(trifluoromethyl)-2,3-dihydro-1H-isoindole (64 mg, 0.271 mmol) in DCM (3 mL) were added 2,4,6-tripropyl-1,3,5,2{5},4{5},6{5}-trioxatriphosphinane 2,4,6-trioxide (50%, 370 μL, 1.25 mmol) and triethylamine (215 μL, 0.652 mmol). The reaction mixture was stirred at ambient temperature for 1 h then water (15 mL) and DCM (10 mL) were added. The layers were separated and the aqueous layer was extracted with DCM (2×15 mL). Combined organic layers were dried over sodium sulfate and evaporated under reduced pressure. The crude residue was purified by flash chromatography over silica gel column (DCM/MeOH) to afford 3-{5-[(1S)-1-methyl-5-(trifluoromethyl)-2,3-dihydro-1H-isoindole-2-carbonyl]-1-oxo-2,3-dihydro-1H-isoindol-2-yl}piperidine-2,6-dione (61 mg, 62% yield) as a white solid. ¹H NMR (500 MHz, DMSO-d6) δ 11.34-10.71 (m, 1H), 7.97-7.77 (m, 2H), 7.76-7.45 (m, 4H), 5.54 (br d, J=6.4 Hz, 1H), 5.21-5.07 (m, 1H), 5.06-4.32 (m, 4H), 2.99-2.56 (m, 2H), 2.48-1.99 (m, 2H), 1.83-0.79 (m, 3H).

### Example 3: Coupling Procedure B: Isoindoline Coupling with CRBN Cores 2, 3, and 4

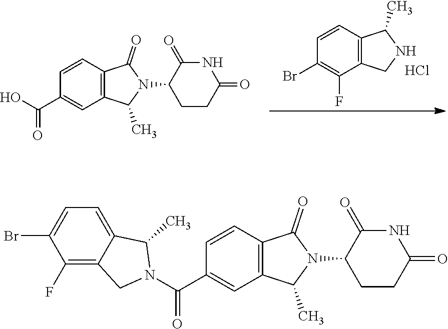 [SMILES: C[C@@H]1c2cc(C(=O)O)ccc2C(=O)N1[C@H]1CCC(=O)NC1=O ; C[C@@H]1NCc2c1ccc(Br)c2F ; Cl ; C[C@@H]1c2cc(C(=O)N3Cc4c(ccc(Br)c4F)[C@@H]3C)ccc2C(=O)N1[C@H]1CCC(=O)NC1=O]

### Representative Example of Coupling Procedure B

To a solution of (3R)-2-[(3S)-2,6-dioxo-3-piperidyl]-3-methyl-1-oxo-isoindoline-5-carboxylic acid (30 mg, 0.10 mmol) and (1S)-5-bromo-4-fluoro-1-methyl-isoindoline; hydrochloride (29 mg, 0.11 mmol) in DCM (0.5 mL). Then were added 2,4,6-tripropyl-1,3,5,2,4,6-trioxatriphosphinane 2,4,6-trioxide (50% in EtOAc, 0.089 mL, 0.15 mmol) and triethylamine (0.07 mL, 0.50 mmol). The RM was stirred at RT for 1 hour then water and DCM were added. The layers were separated and the aqueous layer was extracted with DCM. The combined organic layers were dried over a phase separator then the solvent was evaporated under reduced pressure. The crude residue was purified by flash chromatography over silica gel column (DCM/EtOAc/EtOH) to afford (3S)-3-[(3R)-5-[(1S)-5-bromo-4-fluoro-1-methyl-isoindoline-2-carbonyl]-3-methyl-1-oxo-isoindolin-2-yl]piperidine-2,6-dione (40 mg, 77.3% yield) as a white solid. ¹H NMR (500 MHz, DMSO-d6) δ ppm 10.97 (s, 1H), 7.89 (s, 1H), 7.86-7.48 (m, 5H), 5.63-5.28 (m, 1H), 5.10-4.93 (m, 1H), 4.92-4.73 (m, 2H), 4.59 (d, J=14.92 Hz, 1H), 2.91-2.78 (m, 1H), 2.75-2.56 (m, 2H), 2.10-1.96 (m, 1H), 1.59 (d, J=6.36 Hz, 2H), 1.51-1.41 (m, 3H), 1.12-1.04 (m, 1H).

### Example 3a: Isoindoline Coupling with CRBN Cores 3, and 4

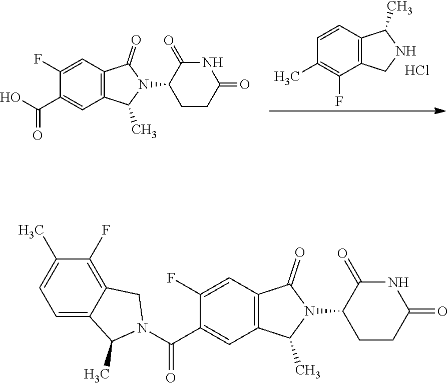 [SMILES: C[C@@H]1c2cc(C(=O)O)c(F)cc2C(=O)N1[C@H]1CCC(=O)NC1=O ; Cc1ccc2c(c1F)CN[C@H]2C ; Cl ; Cc1ccc2c(c1F)CN(C(=O)c1cc3c(cc1F)C(=O)N([C@H]1CCC(=O)NC1=O)[C@@H]3C)[C@H]2C]

To a solution of (3R)-2-[(3S)-2,6-dioxo-3-piperidyl]-6-fluoro-3-methyl-1-oxo-isoindoline-5-carboxylic acid (11 mg, 0.03 mmol) and 1(S)-4-fluoro-1,5-dimethyl-isoindoline hydrochloride (8 mg, 0.04 mmol) in DCM (1.0 mL) were added 2,4,6-tripropyl-1,3,5,2,4,6-trioxatriphosphinane 2,4,6-trioxide (50% in EtOAc, 0.031 mL, 0.05 mmol) and triethylamine (0.028 mL, 0.20 mmol). The RM was stirred at RT for 1 hour then water and EtOAc were added. The layers were separated and the organic layer was washed with saturated NH₄Cl solution then with saturated NaCl solution. The organic layer was dried over Na₂SO₄ then evaporated under reduced pressure. The crude residue was purified by reverse phase flash chromatography (water/MeCN) to afford (3S)-3-[(1R)-5-fluoro-6-[(1S)-4-fluoro-1,5-dimethyl-isoindoline-2-carbonyl]-1-methyl-3-oxo-isoindolin-2-yl]piperidine-2,6-dione (14 mg, 85% yield) as a white solid. ¹H NMR (500 MHz, DMSO-d6) δ ppm 10.98 (d, J=2.93 Hz, 1H), 7.94-7.83 (m, 1H), 7.64 (dd, J=17.24, 8.19 Hz, 1H), 7.32-7.21 (m, 1H), 7.19-6.95 (m, 1H), 5.50-5.03 (m, 1H), 5.03-4.48 (m, 4H), 2.91-2.75 (m, 1H), 2.70-2.57 (m, 2H), 2.21 (s, 3H), 1.55 (d, J=6.36 Hz, 2H), 2.08-1.95 (m, 1H), 1.45 (d, J=6.60 Hz, 3H), 1.11 (d, J=6.36 Hz, 1H).

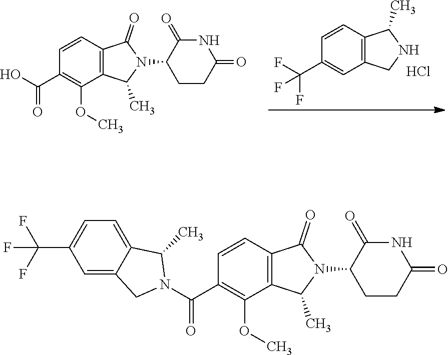

To a solution of (1S)-1-methyl-5-(trifluoromethyl)isoindoline hydrochloride (59 mg, 0.25 mmol), (3R)-2-[(3S)-2,6-dioxo-3-piperidyl]-4-methoxy-3-methyl-1-oxo-isoindoline-5-carboxylic acid (75 mg, 0.23 mmol) and N,N-diethylethanamine (0.16 mL, 1.13 mmol) in DCM (1.1 mL) was added 2,4,6-tripropyl-1,3,5,2,4,6-trioxatriphosphinane 2,4,6-trioxide (50% in EtOAc, 0.20 mL, 0.34 mmol). The reaction mixture was stirred at RT for 30 min then solvent was evaporated under reduced pressure. The crude product was purified by reverse-phase flash chromatography (water/MeCN) to afford (3S)-3-[(3R)-4-methoxy-3-methyl-5-[(1S)-1-methyl-5-(trifluoromethyl)isoindoline-2-carbonyl]-1-oxo-isoindolin-2-yl]piperidine-2,6-dione (50 mg, 43% yield) as a white solid. 1H NMR (500 MHz, DMSO-d6) δ ppm 10.98 (s, 1H), 7.71-7.62 (m, 3H), 7.51-7.50 (m, 2H), 5.50 (q, J=5.79 Hz, 1H), 4.92-4.70 (m, 3H), 4.51 (d, J=15.16 Hz, 1H), 3.95-3.81 (m, 3H), 2.72-2.58 (m, 2H), 2.90-2.80 (m, 1H) 2.05-2.00 (m, 1H), 1.61 (d, J=6.60 Hz, 3H).

### Example 4: Coupling Procedure C: Aminoisoindoline Coupling to CRBN Core 2

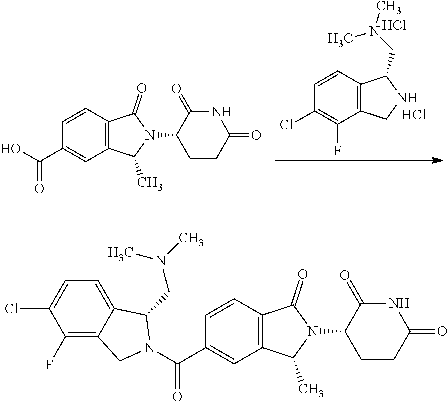

### Representative Example of General Coupling Procedure C

To a solution of (3R)-2-[(3S)-2,6-dioxo-3-piperidyl]-3-methyl-1-oxo-isoindoline-5-carboxylic acid (53 mg, 0.174 mmol) in DCM (1.1 mL) were successively added triethylamine (121 μL, 0.870 mmol), 2,4,6-tripropyl-1,3,5,2{5},4{5},6{5}-trioxatriphosphinane 2,4,6-trioxide (50% in EtOAc, 155 μL, 0.261 mmol) and 1-[(1R)-5-chloro-4-fluoro-isoindolin-1-yl]-N,N-dimethyl-methanamine dihydrochloride (54 mg, 0.179 mmol). The reaction mixture was stirred under nitrogen for 45 minutes then EtOAc and saturated NaHCO₃ solution were added. The layers were separated and the aqueous layer was extracted twice with EtOAc. The combined organic layers were washed with saturated NaCl solution, dried over magnesium sulfate, filtered and concentrated under reduced pressure. The crude residue was purified by reversed phase flash chromatography (water/MeCN) to afford (3S)-3-[(3R)-5-[(1R)-5-chloro-1-[(dimethylamino)methyl]-4-fluoro-isoindoline-2-carbonyl]-3-methyl-1-oxo-isoindolin-2-yl]piperidine-2,6-dione (61 mg, 68% yield) as a white solid. ¹H NMR (600 MHz, DMSO-d6) δ ppm 10.90-11.00 (m, 1H), 7.86 (br s, 1H), 7.72-7.80 (m, 1H), 7.68 (br d, J=7.48 Hz, 1H), 7.50-7.56 (m, 1H), 7.17-7.35 (m, 1H), 5.20-5.55 (m, 1H), 4.98-5.11 (m, 1H), 4.71-4.92 (m, 2H), 4.62 (br d, J=14.38 Hz, 1H), 2.75-2.90 (m, 2H), 2.56-2.71 (m, 3H), 2.25 (s, 4H), 1.97-2.07 (m, 1H), 1.76 (br s, 2H), 1.41-1.50 (m, 3H).

### Example 5: Coupling Procedure D: Isoindoline Coupling to Open CRBN Cores 2, 4 and 5

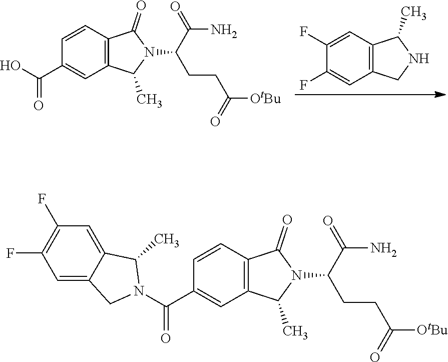 [SMILES: C[C@@H]1c2cc(C(=O)O)ccc2C(=O)N1[C@@H](CCC(=O)OC(C)(C)C)C(N)=O ; C[C@@H]1NCc2cc(F)c(F)cc21 ; C[C@@H]1c2cc(C(=O)N3Cc4cc(F)c(F)cc4[C@@H]3C)ccc2C(=O)N1[C@@H](CCC(=O)OC(C)(C)C)C(N)=O]

### Representative Example of Coupling Procedure D

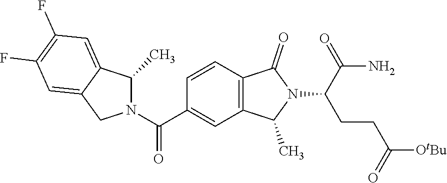 [SMILES: C[C@@H]1c2cc(C(=O)N3Cc4cc(F)c(F)cc4[C@@H]3C)ccc2C(=O)N1[C@@H](CCC(=O)OC(C)(C)C)C(N)=O]

To a solution of (3R)-2-[(1S)-4-tert-butoxy-1-carbamoyl-4-oxo-butyl]-3-methyl-1-oxo-isoindoline-5-carboxylic acid (49 mg, 0.129 mmol) in DCM (1.2 mL) were added T3P 50% in EtOAc (0.16 mL, 0.258 mmol) and triethylamine (0.036 mL, 0.258 mmol). After 5 min, a solution of (1S)-5,6-difluoro-1-methyl-isoindoline hydrochloride (40 mg, 0.194 mmol) and triethylamine (0.036 mL, 0.258 mmol) in DCM (0.5 mL) was added and the reaction mixture was stirred at ambient temperature for 1 hour. EtOAc and saturated NH₄Cl solution were added, the layers were separated and the aqueous layer was extracted with EtOAc. The combined organic extracts were dried over Na₂SO₄ and evaporated under reduced pressure. The crude residue was purified by flash chromatography over silica gel column to afford tert-butyl (4S)-5-amino-4-[(3R)-5-[(1S)-5,6-difluoro-1-methyl-isoindoline-2-carbonyl]-3-methyl-1-oxo-isoindolin-2-yl]-5-oxo-pentanoate (71 mg, 99% yield) as a white solid. ¹H NMR (500 MHz, DMSO-d6) δ 7.84 (d, J=5.8 Hz, 1H), 7.76 (dd, J=8.0, 4.8 Hz, 1H), 7.72-7.61 (m, 1H), 7.57-7.48 (m, 1H), 7.38 (dt, J=43.1, 8.7 Hz, 1H), 7.19 (d, J=31.6 Hz, 2H), 5.31 (dd, J=119.6, 6.8 Hz, 1H), 4.91 (t, J=14.1 Hz, 1H), 4.72 (q, J=6.6 Hz, 1H), 4.44 (t, J=7.1 Hz, 2H), 2.33 (p, J 7.6 Hz, 1H), 2.20 (td, J=9.9, 7.0 Hz, 3H), 1.55 (d, J=6.4 Hz, 2H), 1.44 (d, J=6.6 Hz, 2H), 1.37 (s, 9H), 1.04 (s, 2H), 1.03 (s, 2H).

### Example 6: Cyclization Procedure: Glutarimide Ring Formation with p-TSA

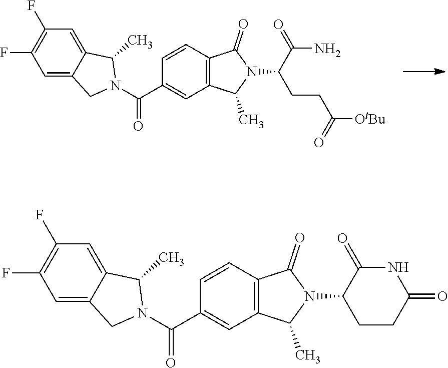 [SMILES: C[C@@H]1c2cc(C(=O)N3Cc4cc(F)c(F)cc4[C@@H]3C)ccc2C(=O)N1[C@@H](CCC(=O)OC(C)(C)C)C(N)=O ; C[C@@H]1c2cc(C(=O)N3Cc4cc(F)c(F)cc4[C@@H]3C)ccc2C(=O)N1[C@H]1CCC(=O)NC1=O]

### Representative Example of Cyclization Procedure

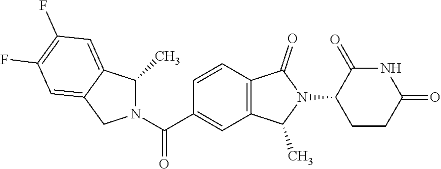 [SMILES: C[C@@H]1c2cc(C(=O)N3Cc4cc(F)c(F)cc4[C@@H]3C)ccc2C(=O)N1[C@H]1CCC(=O)NC1=O]

To a solution of tert-butyl (4S)-5-amino-4-[(3R)-5-[(1S)-5,6-difluoro-1-methyl-isoindoline-2-carbonyl]-3-methyl-1-oxo-isoindolin-2-yl]-5-oxo-pentanoate (71 mg, 0.128 mmol) in acetonitrile (6.4 mL) was added PTSA (49 mg, 0.256 mmol). The reaction mixture was stirred at 85° C. for 16 hours. EtOAc and saturated aqueous NaCl solution were added and the layers were separated. The aqueous layer was extracted with EtOAc. The combined organic layers were dried over Na₂SO₄ and evaporated. The crude residue was purified by flash chromatography over silica gel column (DCM/MeOH) to afford (3S)-3-[(3R)-5-[(1S)-5,6-difluoro-1-methyl-2,3-dihydro-1H-isoindole-2-carbonyl]-3-methyl-1-oxo-2,3-dihydro-1H-isoindol-2-yl]piperidine-2,6-dione (39 mg, 66% yield) as a white solid. ¹H NMR (600 MHz, DMSO-d₆) δ ppm 10.97 (s, 1H), 7.85-7.91 (m, 1H), 7.73-7.79 (m, 1H), 7.64-7.70 (m, 1H), 7.54 (br dd, J=9.98, 7.63 Hz, 1H), 7.33 (br dd, J=9.98, 7.78 Hz, 1H), 5.43 (q, J=5.67 Hz, 1H), 4.87-4.95 (m, 1H), 4.71-4.83 (m, 2H), 4.45 (br d, J=14.53 Hz, 1H), 2.79-2.88 (m, 1H), 2.57-2.71 (m, 2H), 1.97-2.05 (m, 1H), 1.55 (d, J=6.46 Hz, 2H), 1.45 (br d, J=6.75 Hz, 3H).

### Example 7: Isoindoline Synthesis A: Cobalt-Mediated Isoindoline Synthesis

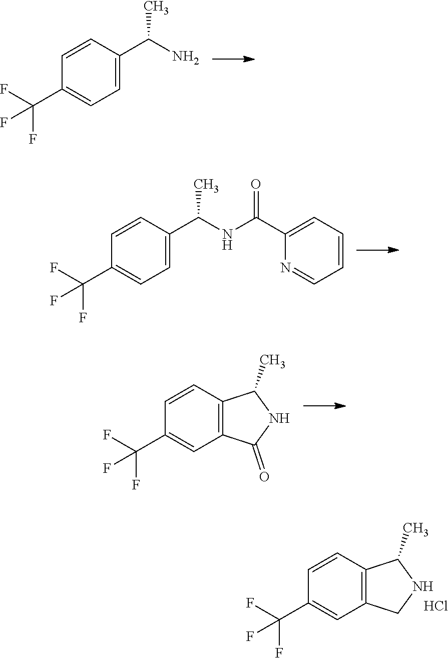 [SMILES: C[C@H](N)c1ccc(C(F)(F)F)cc1 ; C[C@H](NC(=O)c1ccccn1)c1ccc(C(F)(F)F)cc1 ; C[C@@H]1NC(=O)c2cc(C(F)(F)F)ccc21 ; C[C@@H]1NCc2cc(C(F)(F)F)ccc21 ; Cl]

### Representative Example of Isoindoline Synthesis A

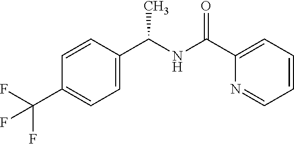 [SMILES: C[C@H](NC(=O)c1ccccn1)c1ccc(C(F)(F)F)cc1]

### N-[rac-(1S)-1-[4-(trifluoromethyl)phenyl]ethyl]pyridine-2-carboxamide

To a solution of pyridine-2-carboxylic acid (11.2 g, 88.2 mmol) in DCM (85 ml) were added HATU (37.6 g, 97.0 mmol) and DIEA (46 mL, 0.265 mol). The mixture was cooled down to 0° C. then (1S)-1-[4-(trifluoromethyl)phenyl]ethanamine (15.0 g, 79.4 mmol) was added dropwise, the reaction was allowed to warm up to ambient temperature and stirred for 16 hours. Water (150 mL) was added and the layers were separated. The organic layer was washed with water (2×150 mL), dried over MgSO₄ and evaporated under reduced pressure. The crude residue was purified by flash chromatography over silica gel column (DCM/MeOH) to afford N-[rac-(1S)-1-[4-(trifluoromethyl)phenyl]ethyl]pyridine-2-carboxamide (20.7 g, 79.8% yield) as a white solid. ¹H NMR (400 MHz DMSO-d6): δ (ppm) 9.22 (d, J=8.3 Hz, 1H), 8.69 (dt, J=4.7, 1.4 Hz, 1H), 8.04-7.95 (m, 2H), 7.73-7.57 (m, 6H), 5.24 (p, J=7.2 Hz, 1H), 1.56 (d, J=7.0 Hz, 3H).

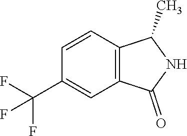

### (3S)-3-methyl-6-(trifluoromethyl)isoindolin-1-one

An autoclave was charged with N-[(1S)-1-[4-(trifluoromethyl)phenyl]ethyl]pyridine-2-carboxamide (20.7 g, 70.3 mmol), 2,2-dimethylpropanoic acid (14.4 g, 0.141 mol), silver carbonate (39.1 g, 0.141 mol), cobalt(2+) acetate hydrate (1:2:4) (3.5 g, 14.1 mmol) 2,2,2-Trifluoroethanol (120 mL) and N-[(1S)-1-[4-(trifluoromethyl)phenyl]ethyl]pyridine-2-carboxamide (20.7 g, 70.3 mmol). The reaction mixture was stirred for 16 hours at 110° C. then cooled down to ambient temperature and the mixture was filtered over dicalite. The filtrate was evaporated under reduced pressure and the crude residue was dissolved in iPrOAc (200 mL). The organic layer was washed with saturated NaHCO₃ solution (200 mL) then saturated NaCl solution (200 mL), dried over MgSO₄ and the solvent was evaporated under reduced pressure. The crude residue was purified by flash chromatography over silica gel column (DCM/MeOH) to afford (3S)-3-methyl-6-(trifluoromethyl)isoindolin-1-one (11.4 g, 70.1% yield) as an orange solid. ¹H NMR (400 MHz, DMSO-d6): δ (ppm) 8.93 (s, 1H), 7.98 (dd, J=7.9, 1.8 Hz, 1H), 7.92-7.82 (m, 2H), 4.75 (q, J=6.8 Hz, 1H), 1.41 (d, J=6.8 Hz, 3H).

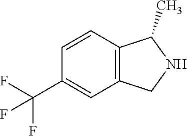

### (1S)-1-methyl-5-(trifluoromethyl)isoindoline

To a solution of rac-(3S)-3-methyl-6-(trifluoromethyl)isoindolin-1-one (1.9 g, 8.83 mmol) in THF (40 mL) under nitrogen atmosphere, was added NaBH₄ (2.4 g, 61.8 mmol). The mixture was cooled down to 0° C. and BF₃ etherate (8.9 mL, 70.6 mmol) was added. The reaction mixture was heated to 70° C. for 16 hours then cooled down to 0° C. HCl 1N in water (25 mL) was added and the mixture was stirred for 30 minutes at ambient temperature. The mixture was extracted with iPrOEt (2×25 mL), the aqueous layer was basified with NaOH and extracted twice with iPrOEt (2×25 mL). Combined organic layers were dried over MgSO₄ and evaporated under reduced pressure. The crude residue was purified by flash chromatography over silica gel column (DCM/MeOH) to afford (1S)-1-methyl-5-(trifluoromethyl)isoindoline (1.5 g, 82.0% yield) as a light pink solid. ¹H NMR (400 MHz, DMSO-d6): δ (ppm) 9.55 (s, 1H), 9.23 (s, 1H), 7.84 (s, 1H), 7.81-7.76 (m, 1H), 7.64 (d, J=8.0 Hz, 1H), 5.02 (d, J=8.4 Hz, 1H), 4.59 (d, J=7.2 Hz, 2H), 1.60 (d, J=6.8 Hz, 3H).

### Example 8: Isoindoline Synthesis B: Isoindoline Synthesis Via Chloro Intermediate

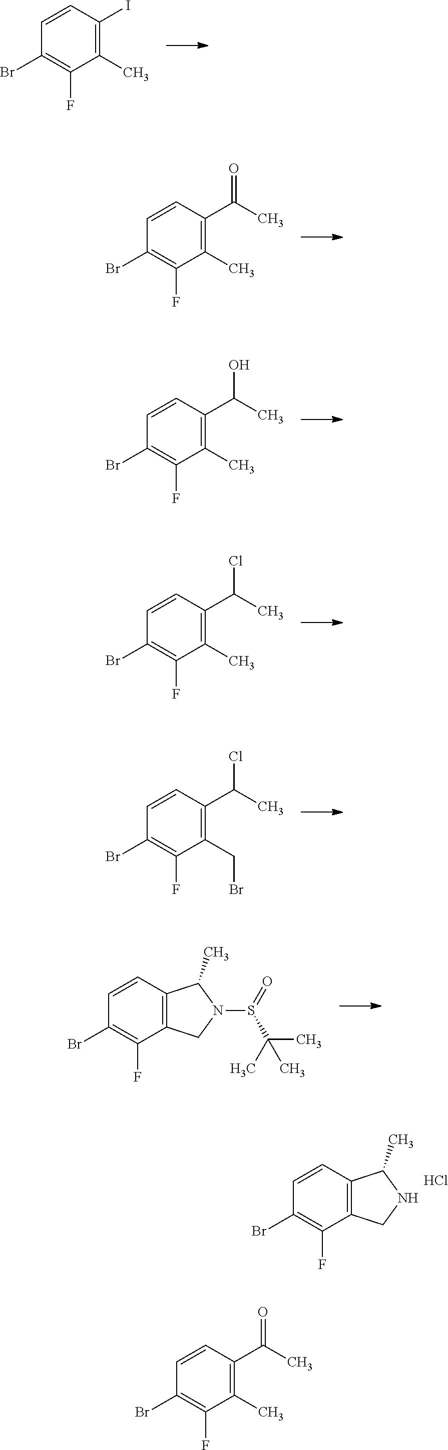

### 1-(4-bromo-3-fluoro-2-methyl-phenyl)ethanone

To a solution of 1-bromo-2-fluoro-4-iodo-3-methylbenzene (6.6 mL, 43.3 mmol) in 1,4-dioxane (100 mL) were added tributyl(1-ethoxyvinyl)stannane (17 mL, 47.6 mmol) and dichloropalladium,triphenylphosphane (1.5 g, 2.16 mmol). The reaction mixture was purged with nitrogen and heated to 90° C. for 4.5 hours. HCl 1N in water (15 mL) was added and the solution was stirred for 30 minutes at ambient temperature then the solvent was evaporated under reduced pressure. EtOAc (150 mL) and water (150 mL) were added, the layers were separated and the organic layer was washed with NH₄Cl saturated solution (150 mL), saturated NaHCO₃ solution (150 mL) and saturated NaCl solution (150 mL). The combined organic layers were dried over sodium sulfate and the solvent was evaporated under reduced pressure. The crude residue was purified by flash chromatography over silica gel (CyHex/EtOAc) to afford 1-(4-bromo-3-fluoro-2-methyl-phenyl)ethanone (8.93 g, 89.3% yield) as a white solid. 1H NMR (400 MHz, DMSO-d6): δ (ppm) 7.68 (dd, J=8.4, 6.7 Hz, 1H), 7.58 (dd, J=8.3, 1.3 Hz, 1H), 2.56 (s, 3H), 2.34 (d, J=2.7 Hz, 3H).

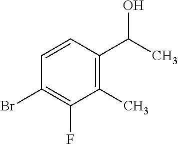

### 1-(4-bromo-3-fluoro-2-methyl-phenyl)ethanol

To a solution of 1-(4-bromo-3-fluoro-2-methyl-phenyl)ethanone (6.9 g, 29.8 mmol) in methanol (70 mL) maintained at −40° C. was added slowly sodium borohydride (1.2 g, 32.8 mmol). The reaction mixture was allowed to warm up to ambient temperature over 2 hours. Saturated ammonium chloride solution (50 mL) was added and the solvent was evaporated under reduced pressure. The residue was diluted in EtOAc (50 mL) and the organic layer was washed with saturated ammonium chloride solution (25 mL) then saturated sodium carbonate solution (25 mL) and saturated NaCl solution (25 mL). The combined organic layers were dried over sodium sulfate then evaporated under reduced pressure. The crude residue was purified by flash chromatography over silica gel to afford 1-(4-bromo-3-fluoro-2-methyl-phenyl)ethanol (6.16 g, 89%) as a colorless oil. 1H NMR (400 MHz, DMSO-d6): δ (ppm) 7.49 (dd, J=8.4, 7.2 Hz, 1H), 7.25 (dd, J=8.4, 1.2 Hz, 1H), 5.26 (d, J=4.2 Hz, 1H), 4.89 (qd, J=6.3, 4.1 Hz, 1H), 2.23 (d, J=2.7 Hz, 3H), 1.27 (d, J=6.4 Hz, 3H).

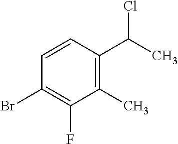

### 1-bromo-4-(1-chloroethyl)-2-fluoro-3-methyl-benzene

To a solution of 1-bromo-4-(1-chloroethyl)-2-fluoro-3-methyl-benzene (9.5 g, 37.8 mmol) in DCE (80 mL) were added 1-bromopyrrolidine-2,5-dione (10.2 g, 56.7 mmol) and 2-[({E})-(1-cyano-1-methyl-ethyl)azo]-2-methyl-propanenitrile (633 mg, 3.78 mmol). The reaction mixture was stirred at 80° C. for 2 days then saturated aqueous NaHCO₃ solution (80 mL) was added. The layers were separated and the organic layer was washed with saturated NaCl (50 mL), dried over sodium sulfate and evaporated under reduced pressure. The crude residue was purified by flash chromatography over silica gel column to afford 1-bromo-4-(1-chloroethyl)-2-fluoro-3-methyl-benzene (8.7 g, 91.2% yield) as a colorless oil. ¹H NMR (400 MHz, DMSO-d6): δ (ppm) 7.67-7.54 (m, 1H), 7.36 (dd, J=8.5, 1.2 Hz, 1H), 5.53 (q, J=6.7 Hz, 1H), 2.33 (d, J=2.6 Hz, 3H), 1.81 (d, J=6.8 Hz, 3H).

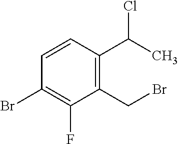

### 1-bromo-3-(bromomethyl)-4-(1-chloroethyl)-2-fluoro-benzene

To a solution of 1-bromo-4-(1-chloroethyl)-2-fluoro-3-methyl-benzene (1.15 g, 4.57 mmol) in DCE (23 mL) were added 1-bromopyrrolidine-2,5-dione (1.2 g, 6.86 mmol) and 2-[({E})-(1-cyano-1-methyl-ethyl)azo]-2-methyl-propanenitrile (75 mg, 0.457 mmol). The reaction was stirred at 80° C. for 2 hours then the solvent was evaporated under reduced pressure and EtOAc was added. The solution was washed with saturated NaHCO₃ solution then saturated sodium bisulfite solution. The organic layer was dried over sodium sulfate and the solvent was evaporated under reduced pressure. The crude residue was purified by flash chromatography over silica gel column to afford 1-bromo-3-(bromomethyl)-4-(1-chloroethyl)-2-fluoro-benzene (1.3 g, 87.4% yield) as a colorless oil. 1H NMR (500 MHz, DMSO-d6) δ 7.78 (dd, J=8.6, 7.2 Hz, 1H), 7.47 (dd, J=8.6, 1.3 Hz, 1H), 5.65 (q, J=6.7 Hz, 1H), 4.90-4.76 (m, 2H), 1.82 (d, J=6.6 Hz, 3H).

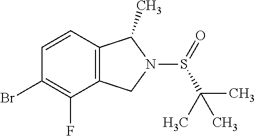 [SMILES: C[C@H]1c2ccc(Br)c(F)c2CN1[S@](=O)C(C)(C)C]

### 5-bromo-2-tert-butylsulfinyl-4-fluoro-1-methyl-isoindoline

In a round bottom flask were added 1-bromo-3-(bromomethyl)-4-(1-chloroethyl)-2-fluoro-benzene (2.8 g, 8.41 mmol), ({R})-2-methylpropane-2-sulfinamide (2.3 g, 18.5 mmol), potassium hydroxide (567 mg, 10.1 mmol) and THF (42 mL). The reaction mixture was stirred at ambient temperature for 10 days then diluted with EtOAc. The solution was washed with water then saturated ammonium chloride solution and saturated NaCl. The organic layer was dried over Na₂SO₄, filtered and the solvent was evaporated under reduced pressure. The crude residue was purified by flash chromatography over silica gel column then the diastereomers were separated by preparative SFC (Instrument: Waters Prep SFC200. Stationary Phase: Pirkle (R,R) Whelk-01 5 μm, 250×21.1 mm. Mobile layer: CO2/MeOH 80/20. Flow rate: 50 mL/minutes. UV detection: λ=210 nm. Temperature: 40° C. Pressure: 100 bars) to afford:

(1S)-5-bromo-2-[(R)-tert-butylsulfinyl]-4-fluoro-1-methyl-isoindoline (1.6 g, 20% yield); ¹H NMR (400 MHz, DMSO-d6): δ (ppm) 7.60 (dd, J=8.0, 6.4 Hz, 1H), 7.14 (d, J=8.5 Hz, 1H), 4.96 (qd, J=6.6, 1.8 Hz, 1H), 4.83-4.74 (m, 1H), 4.51 (d, J=15.0 Hz, 1H), 1.44 (d, J=6.6 Hz, 3H), 1.19 (s, 9H); and

(1R)-5-bromo-2-[(R)-tert-butylsulfinyl]-4-fluoro-1-methyl-isoindoline (2.1 g, 26% yield); ¹H NMR (400 MHz, DMSO-d6): δ (ppm) 7.62 (dd, J=8.0, 6.5 Hz, 1H), 7.11 (dd, J=8.2, 0.9 Hz, 1H), 4.96 (qd, J=6.6, 2.2 Hz, 1H), 4.82 (dd, J=14.5, 1.1 Hz, 1H), 4.23 (dd, J=14.5, 2.5 Hz, 1H), 1.40 (d, J=6.5 Hz, 3H), 1.19 (s, 9H).

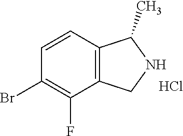

### (1R)-5-bromo-4-fluoro-1-methyl-isoindoline Hydrochloride

To a solution of (1S)-5-bromo-2-[(R)-tert-butylsulfinyl]-4-fluoro-1-methyl-isoindoline (50 mg, 0.150 mmol) in DCM (2 mL) at 0° C. was added HCl 4 M in dioxane (0.37 mL, 1.50 mmol). The reaction mixture was stirred at ambient temperature for 2 hours then the solvent was evaporated under reduced pressure. Saturated NaHCO₃ solution and EtOAc were added, the layers were separated and the aqueous layer was extracted with EtOAc. The combined organic layers were dried on Na₂SO₄ and the solvent was evaporated under reduced pressure. HCl 4N in dioxane was added and the solid was filtered to afford (1R)-5-bromo-4-fluoro-1-methyl-isoindoline hydrochloride (237 mg, 97%) yield) as light pink solid. ¹H NMR (400 MHz, DMSO-d6) δ 10.25 (s, 1H), 9.82 (s, 1H), 7.76 (dd, J=8.1, 6.4 Hz, 1H), 7.23 (dd, J 8.1, 0.9 Hz, 1H), 4.96 (q, J=6.8 Hz, 1H), 4.63 (q, J=14.8 Hz, 2H), 1.59 (d, J=6.9 Hz, 3H).

### Example 9: Isoindoline Synthesis C: Isoindoline Synthesis Via Ketone Intermediate

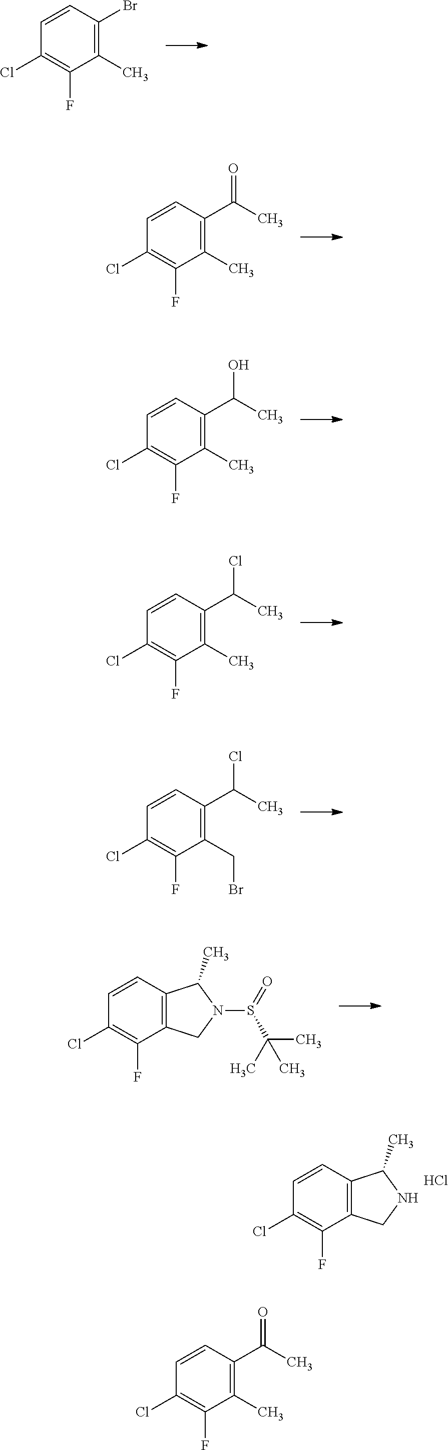

### 1-(4-chloro-3-fluoro-2-methyl-phenyl)ethenone

To a solution of 1-bromo-4-chloro-3-fluoro-2-methylbenzene (20.0 g, 87.7 mmol) in dioxane (200 mL) were added tributyl(1-ethoxyvinyl)stannane (34 mL, 96.5 mmol) and dichloropalladium,triphenylphosphane (3.08 g, 4.39 mmol). The mixture was purged with nitrogen and stirred at 90° C. for 16 hours. Aqueous 1N HCl (40 mL) was added and the solution was stirred for 30 minutes at ambient temperature. The dioxane was evaporated under reduced pressure and EtOAc (100 mL) was added. The organic layer was washed with brine (50 mL) then dried over sodium sulfate, filtered and evaporated under reduced pressure. The crude residue was purified flash chromatography over silica gel column (Hept/EtOAc) to afford 1-(4-chloro-3-fluoro-2-methyl-phenyl)ethanone (16.3 g, 99.6% yield) as a colorless oil. ¹H NMR (400 MHz, DMSO-d6) δ 7.67 (dd, J=8.4, 1.3 Hz, 1H), 7.57 (ddd, J=8.2, 7.2, 0.7 Hz, 1H), 2.57 (s, 3H), 2.35 (d, J=2.7 Hz, 3H).

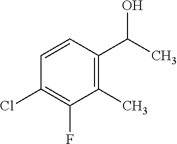

### 1-(4-chloro-3-fluoro-2-methyl-phenyl)ethanol

To a solution of 1-(4-chloro-3-fluoro-2-methyl-phenyl)ethanone (16.3 g, 87.3 mmol) in methanol (190 mL) at −78° C. was slowly added sodium borohydride (3.63 g, 96.1 mmol). The reaction mixture was allowed to warm up to ambient temperature for 2 hours then saturated NH₄Cl (5 ml) solution was added and the layers were separated. The methanol was evaporated under reduced pressure and the residue was diluted in IprOAc. The organic layer was washed with saturated NH₄Cl solution, saturated NaHCO₃ solution and brine to afford 1-(4-chloro-3-fluoro-2-methyl-phenyl)ethanol (15.8 g, 95.9% yield) as a colorless oil. ¹H NMR (400 MHz, DMSO-d6) δ 7.42-7.34 (m, 1H), 7.31 (dd, J=8.5, 1.1 Hz, 1H), 5.26 (d, J=4.3 Hz, 1H), 4.90 (qd, J=6.4, 4.2 Hz, 1H), 2.22 (d, J=2.5 Hz, 3H), 1.28 (d, J=6.4 Hz, 3H).

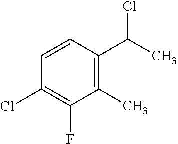

### 1-chloro-4-(1-chloroethyl)-2-fluoro-3-methyl-benzene

To a solution of 1-(4-chloro-3-fluoro-2-methyl-phenyl)ethanol (15.80 g, 83.8 mmol) in DCM (190 mL) was added thionyl chloride (12 mL, 0.168 mol) at 0° C. The reaction mixture was allowed to warm up to ambient temperature and stirred for 4 hours. The solvent was evaporated under reduced pressure and the crude residue was dissolved in EtOAc/water (1/1: 50 mL). The layers were separated and the aqueous layer was washed with EtOAc (2×25 mL). The combined organic layers were dried over sodium sulfate and evaporated under reduced pressure to afford 1-chloro-4-(1-chloroethyl)-2-fluoro-3-methyl-benzene (15.2 g, 87.6% yield) as a yellow oil. ¹H NMR (400 MHz, DMSO-d6) δ 7.48-7.37 (m, 2H), 5.54 (q, J=6.7 Hz, 1H), 2.33 (d, J=2.5 Hz, 3H), 1.81 (d, J=6.7 Hz, 3H).

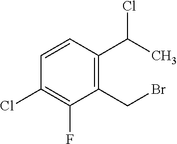

### 3-(bromomethyl)-1-chloro-4-(1-chloroethyl)-2-fluoro-benzene

To a of 1-chloro-4-(1-chloroethyl)-2-fluoro-3-methyl-benzene (12.7 g, 61.3 mmol) in acetonitrile (77 mL) at ambient temperature under a nitrogen atmosphere was added 1-bromopyrrolidine-2,5-dione (16.4 g, 92.0 mmol). The reaction mixture was stirred under LED visible light irradiation at 75° C. for 3 hours then EtOAc (40 mL) was added. The layers were separated and the organic layer was washed with saturated Na₂S₂O₃ solution (50 mL), dried over MgSO₄ and evaporated under reduced pressure. The crude residue was purified by flash chromatography over silica gel column (CyHex/EtOAc) to afford 3-(bromomethyl)-1-chloro-4-(1-chloroethyl)-2-fluoro-benzene (15.0 g, 85.5% yield) as a light yellow oil. ¹H NMR (400 MHz, DMSO-d6) δ 7.68 (dd, J=8.6, 7.7 Hz, 1H), 7.54 (dd, J=8.6, 1.4 Hz, 1H), 5.66 (q, J=6.7 Hz, 1H), 4.90-4.80 (m, 2H), 1.82 (dd, J=6.7, 4.2 Hz, 3H).

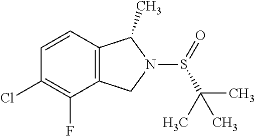 [SMILES: C[C@H]1c2ccc(Cl)c(F)c2CN1[S@](=O)C(C)(C)C]

### (1S)-2-[(R)-tert-butylsulfinyl]-5-chloro-4-fluoro-1-methyl-isoindoline

To a solution of 3-(bromomethyl)-1-chloro-4-(1-chloroethyl)-2-fluoro-benzene (6.00 g, 21.0 mmol) and ({R})-2-methylpropane-2-sulfinamide (98%, 5.71 g, 46.2 mmol) in THF (210 mL) was added sodium hydride (60% in grease, 2.77 g, 69.2 mmol). The reaction mixture was stirred at ambient temperature for 1 hour then IprOH (20 mL) was added and the reaction mixture was diluted with EtOAc (100 mL). The layers were separated and the organic layer was washed with brine (100 mL), dried over sodium sulfate and evaporated under reduced pressure. The crude residue was purified by flash chromatography over silica gel column (CyHex/EtOAC) to afford 2-[(R)-tert-butylsulfinyl]-5-chloro-4-fluoro-1-methyl-isoindoline (3.0 g, 49.3% yield) as a pink oil. The diastereomeric mixture was purified by SFC (Instrument: Waters Prep SFC200. Stationary Phase: Pirkle (R,R) Whelk-01 5 μm, 250×21.1 mm. Mobile phase: CO2/MeOH 80/20. Flow rate: 50 mL/min. UV detection: k=210 nm. Temperature: 40° C. Pressure: 100 bars. Sample: batch was dissolved by sonication in MeOH to a concentration of 124 mg/mL. This solution was filtered on GHP 0.45 μm filter. Collection conditions: UV threshold:—C1 start 1000 mAU; stop 250 mAU—C2 start 1200 mAU; stop 200 mAU. Stacking conditions: Interval: 7.03 min. Elution time: 10 min. Injections: 3 mL (371.4 mg) to afford (1S)-2-[(R)-tert-butylsulfinyl]-5-chloro-4-fluoro-1-methyl-isoindoline (2.1 g, 35.0% yield) as a light pink oil. ¹H NMR (400 MHz, DMSO-d6) δ 7.49 (dd, J=8.0, 6.8 Hz, 1H), 7.20 (d, J=8.0 Hz, 1H), 5.03-4.89 (m, 1H), 4.79 (d, J=15.1 Hz, 1H), 4.51 (d, J=15.0 Hz, 1H), 1.44 (d, J=6.7 Hz, 3H), 1.19 (s, 9H).

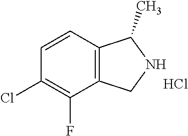

### (1S)-5-chloro-4-fluoro-1-methyl-isoindoline Hydrochloride

A solution of (1S)-2-[(R)-tert-butylsulfinyl]-5-chloro-4-fluoro-1-methyl-isoindoline (2.20 g, 7.59 mmol) in hydrogen chloride (4M in dioxane, 38 mL, 0.152 mol) was stirred at ambient temperature for 2 h. The reaction mixture was poured into Et₂O (250 mL). The suspension was filtered, washed with Et₂O (2×10 mL) and dried under reduced pressure to afford (1S)-5-chloro-4-fluoro-1-methyl-isoindoline hydrochloride (1.6 g, 94.9% yield) as a white powder. ¹H NMR (400 MHz, DMSO-d6) δ 9.81 (s, 2H), 7.65 (dd, J=8.1, 6.9 Hz, 1H), 7.30 (d, J=8.0 Hz, 1H), 4.98 (q, J=6.8 Hz, 1H), 4.72-4.54 (m, 2H), 1.58 (d, J=6.8 Hz, 3H).

### Example 10: Isoindoline Synthesis D: Isoindoline Synthesis Via Lithiation

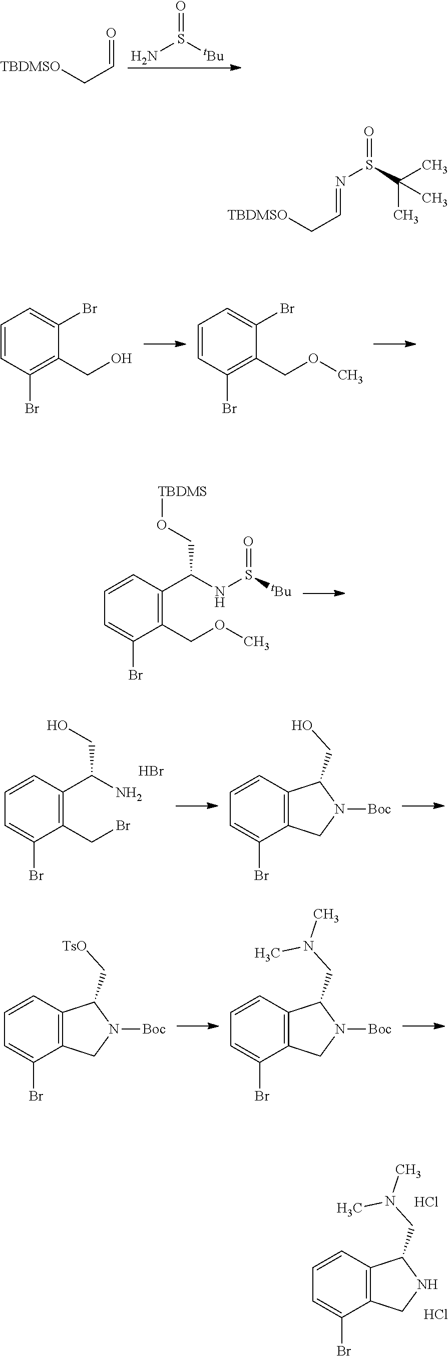

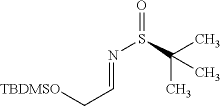 [SMILES: CC(C)(C)[S@](=O)/N=C/CO[Si](C)(C)C(C)(C)C]

### (NE,S)—N-[2-[tert-butyl(dimethyl)silyl]oxyethylidene]-2-methyl-propane-2-sulfinamide

To a stirred solution of {[tert-butyl(dimethyl)silyl]oxy}acetaldehyde (10.0 g, 51.63 mmol) in toluene (75 mL) at ambient temperature under nitrogen were added (S)-(−)-2-Methyl-2-propanesulfinamide (7.0 g, 56.79 mmol) and copper sulfate (16.7 g, 103.3 mmol). The reaction mixture was stirred at 45° C. overnight then filtered over Celite. The filtrate was concentrated under reduced pressure. The crude residue was purified by flash chromatography over silica gel column (CyHex/EtOAc) to afford (NE,S)—N-[2-[tert-butyl(dimethyl)silyl]oxyethylidene]-2-methyl-propane-2-sulfinamide (7.65 g, 51.8% yield) as a colorless oil. 1H NMR (400 MHz, DMSO-d6) δ 7.90 (t, J=2.7 Hz, 1H), 4.60 (d, J=2.7 Hz, 2H), 1.12 (s, 9H), 0.88 (s, 9H), 0.07 (s, 6H).

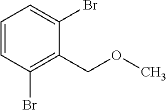

### 1,3-dibromo-2-(methoxymethyl)benzene

To a solution of (2,6-dibromophenyl)methanol (10.0 g, 35.7 mmol) in THF (90 mL) at 0° C. under nitrogen was added sodium hydride (1.57 g, 39.3 mmol). The reaction mixture was stirred at 0° C. for 15 minutes then iodomethane (6.67 mL, 107.2 mmol) was added. The reaction mixture was stirred at ambient temperature for 3 hours. The reaction mixture was quenched with saturated aqueous NH4Cl solution then EtOAc was added. The layers were separated and the organic layer was washed with brine, dried over sodium sulfate, filtered and evaporated under reduced pressure. The crude material was purified by flash chromatography over silica gel column (CyHex/EtOAc) to afford 1,3-dibromo-2-(methoxymethyl)benzene (9.82 g, 98.2% yield) as a white solid. 1H NMR (400 MHz, CDCl3) δ 7.55 (d, J=8.1 Hz, 2H), 7.02 (t, J=8.0 Hz, 1H), 4.77 (s, 2H), 3.46 (s, 3H).

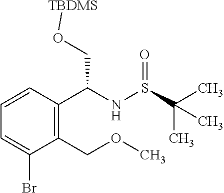

### (S)—N-[(1R)-1-[3-bromo-2-(methoxymethyl)phenyl]-2-[tert-butyl(dimethyl)silyl]oxy-ethyl]-2-methyl-propane-2-sulfinamide

To a solution of 1,3-dibromo-2-(methoxymethyl)benzene (4.6 g, 16.43 mmol) in THF (70 mL) at −78° C. under nitrogen was added 1.6 M butyllithium (10.8 mL, 17.25 mmol). The reaction mixture was stirred at −78° C. for 10 minutes then a solution of (S)—N-[2-[tert-butyl(dimethyl)silyl]oxyethylidene]-2-methyl-propane-2-sulfinamide (5.02 g, 18.07 mmol) in THF (15 mL) was added. The reaction mixture was stirred at ambient temperature for 5 minutes. The reaction mixture was quenched with saturated NH₄Cl solution and EtOAc was added. The layers were separated and the organic layers were washed with saturated NaCl solution, dried over sodium sulfate, filtered and concentrated under reduced pressure. The crude residue was purified by flash chromatography over silica gel column (CyHex/EtOAc) to afford:

(S)—N-[(1S)-1-[3-bromo-2-(methoxymethyl)phenyl]-2-[tert-butyl(dimethyl)silyl]oxy-ethyl]-2-methyl-propane-2-sulfinamide (2.37 g, 30.1 yield) as a colorless oil; ¹H NMR (400 MHz, CDCl3) δ 7.53 (dd, J=8.0, 1.3 Hz, 1H), 7.42 (dd, J=7.9, 1.3 Hz, 1H), 7.16 (t, J=7.9 Hz, 1H), 4.91-4.82 (m, 1H), 4.76 (q, J=10.8 Hz, 2H), 4.39 (d, J=1.7 Hz, 1H), 3.87 (dd, J=10.0, 4.1 Hz, 1H), 3.62-3.52 (m, 1H), 3.45 (s, 3H), 1.21 (s, 9H), 0.91 (s, 11H), 0.07 (d, J=13.4 Hz, 6H); and

(S)—N-[(1R)-1-[3-bromo-2-(methoxymethyl)phenyl]-2-[tert-butyl(dimethyl)silyl]oxy-ethyl]-2-methyl-propane-2-sulfinamide (4.12 g, 52.4% yield) as a colorless oil; ¹H NMR (400 MHz, CDCl3) δ 7.52 (ddd, J=9.1, 7.9, 1.2 Hz, 2H), 7.18 (t, J=7.9 Hz, 1H), 4.82 (dt, J=6.0, 4.6 Hz, 1H), 4.78-4.66 (m, 2H), 4.28 (d, J=4.5 Hz, 1H), 3.86 (dd, J=10.1, 4.8 Hz, 1H), 3.79 (dd, J=10.1, 6.1 Hz, 1H), 3.42 (s, 3H), 1.20 (s, 9H), 0.85 (s, 9H), −0.03 (d, J=6.2 Hz, 6H).

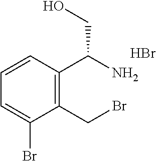 [SMILES: N[C@@H](CO)c1cccc(Br)c1CBr ; Br]

### (2R)-2-amino-2-[3-bromo-2-(bromomethyl)phenyl]ethanol hydrobromide

To a stirred solution of (S)—N-[(1R)-1-[3-bromo-2-(methoxymethyl)phenyl]-2-[tert-butyl(dimethyl)silyl]oxy-ethyl]-2-methyl-propane-2-sulfinamide (4.12 g, 8.61 mmol) in DCM (34 mL) at 0° C. under nitrogen was added boron tribromide 1 M in DCM (9.47 mL, 9.47 mmol). The reaction mixture was allowed to warm up to ambient temperature and stirred for 1.5 hour. The reaction mixture was concentrated under reduced pressure then the crude residue was taken up in MeOH and concentrated under reduced pressure to afford (2R)-2-amino-2-[3-bromo-2-(bromomethyl)phenyl]ethanol hydrobromide (3.3 g, 98.3% yield) as an orange solid.

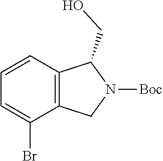 [SMILES: CC(C)(C)OC(=O)N1Cc2c(Br)cccc2[C@@H]1CO]

### tert-butyl (1R)-4-bromo-1-(hydroxymethyl)isoindoline-2-carboxylate

A solution of (2R)-2-amino-2-[3-bromo-2-(bromomethyl)phenyl]ethanol hydrobromide (3.54 g, 9.08 mmol) in dioxane (250 mL) and water (25 mL) and a solution of triethylamine (25.3 mL, 181.59 mmol) in dioxane (259 mL) were passed through a 10 mL reactor at 150° C. with a residence time of 2.5 minutes. The mixture was collected in a solution of tert-butoxycarbonyl tert-butyl carbonate (2.50 g, 11.45 mmol) in dioxane (50 mL) at ambient temperature. The resulting solution was concentrated under reduced pressure and the residue was taken up in EtOAc (100 mL) then washed with saturated NH₄Cl solution (2×50 mL), dried over sodium sulfate, filtered and concentrated under reduced pressure. The crude residue was purified by flash chromatography over silica gel column (DCM/MeOH) to afford tert-butyl (1R)-4-bromo-1-(hydroxymethyl)isoindoline-2-carboxylate (1.8 g, 60.4% yield) as a pink oil. 1H NMR (400 MHz, DMSO-d6) δ 7.49 (d, J=7.8 Hz, 1H), 7.38 (t, J=7.0 Hz, 1H), 7.25 (t, J=7.7 Hz, 1H), 5.05-4.91 (m, 1H), 4.77 (dt, J=10.7, 5.6 Hz, 1H), 4.57 (dd, J=15.2, 3.1 Hz, 1H), 4.41 (ddd, J=17.8, 15.0, 2.6 Hz, 1H), 3.82-3.65 (m, 2H), 1.46 (d, J=3.5 Hz, 9H).

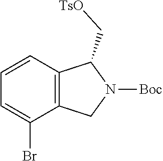 [SMILES: Cc1ccc(S(=O)(=O)OC[C@H]2c3cccc(Br)c3CN2C(=O)OC(C)(C)C)cc1]

### tert-butyl (1R)-4-bromo-1-(p-tolylsulfonyloxymethyl)isoindoline-2-carboxylate

To a solution of tert-butyl (1R)-4-bromo-1-(hydroxymethyl)isoindoline-2-carboxylate (250 mg, 0.76 mmol) in DCM (3 mL) at 0° C. under nitrogen were added DMAP (9 mg, 0.08 mmol), triethylamine (0.32 mL, 2.29 mmol) and 4-methylbenzenesulfonyl chloride (218 mg, 1.14 mmol). The reaction mixture was allowed to warm up to ambient temperature and stirred for 2 hours. The reaction mixture was quenched with saturated NH₄Cl solution (25 mL) and EtOAc (25 mL) was added. The layers were separated and the aqueous layer was extracted with EtOAc (2×25 mL). The combined organic layers were dried over sodium sulfate, filtered and concentrated under reduced pressure. The crude residue was purified by flash chromatography over silica gel column (CyHex/EtOAc) to afford tert-butyl (1R)-4-bromo-1-(p-tolylsulfonyloxymethyl)isoindoline-2-carboxylate (310 mg, 84.4% yield) as a white solid. ¹H NMR (400 MHz, DMSO-d6) δ 7.61 (dd, J=15.8, 8.1 Hz, 2H), 7.54 (d, J=7.8 Hz, 1H), 7.43 (t, J=6.7 Hz, 2H), 7.31 (d, J=7.7 Hz, 1H), 7.25 (t, J=7.2 Hz, 1H), 5.24 (s, 1H), 4.51 (d, J=15.0 Hz, 2H), 4.43 (t, J=9.5 Hz, 2H), 4.26 (d, J=15.2 Hz, 1H), 2.41 (s, 3H), 1.45-1.25 (m, 9H).

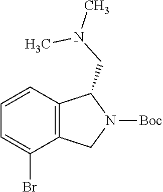 [SMILES: CN(C)C[C@H]1c2cccc(Br)c2CN1C(=O)OC(C)(C)C]

### tert-butyl (1R)-4-bromo-1-[(dimethylamino)methyl]isoindoline-2-carboxylate

A mixture of tert-butyl (1R)-4-bromo-1-(p-tolylsulfonyloxymethyl)isoindoline-2-carboxylate (110 mg, 0.23 mmol) and 2M N-methylmethanamine (4.6 mL, 9.12 mmol) was stirred at 70° C. for 16 hours. Saturated NaHCO₃ solution (15 mL) and EtOAc (15 mL) were added, the layers were separated and the aqueous layer was extracted with EtOAc (2×15 mL). The combined organic layers were dried over sodium sulfate, filtered and concentrated under reduced pressure to afford tert-butyl (1R)-4-bromo-1-[(dimethylamino)methyl]isoindoline-2-carboxylate (80 mg, 98.7% yield) as an orange oil. 1H NMR (400 MHz, DMSO-d6) δ 7.49 (d, J=7.9 Hz, 1H), 7.42 (d, J=7.5 Hz, 1H), 7.25 (t, J=7.8 Hz, 1H), 5.00 (d, J=8.3 Hz, 1H), 4.58 (d, J=15.2 Hz, 1H), 4.42 (t, J=16.7 Hz, 1H), 2.17 (d, J=9.9 Hz, 6H), 1.47 (s, 9H).

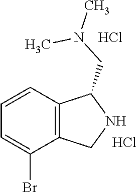

### 1-[(1R)-4-bromoisoindolin-1-yl]-N,N-dimethyl-methanamine Dihydrochloride

A solution of tert-butyl (1R)-4-bromo-1-[(dimethylamino)methyl]isoindoline-2-carboxylate (80 mg, 0.225 mmol) and 4M hydrogen chloride (1.1 mL, 4.50 mmol) in dioxane was stirred at ambient temperature for 1 hour. The reaction mixture was diluted with Et₂O and filtered to afford 1-[(1R)-4-bromoisoindolin-1-yl]-N,N-dimethyl-methanamine dihydrochloride as a blue solid (73 mg, 98.8% yield). 1H NMR (400 MHz, DMSO-d6) δ 10.37 (s, 4H), 7.68 (d, J=7.9 Hz, 1H), 7.52 (d, J=7.7 Hz, 1H), 7.42 (t, J=7.8 Hz, 1H), 5.54 (s, 1H), 4.57 (q, J=15.0 Hz, 2H), 2.93 (s, 6H).

### Example 11: Isoindoline Synthesis E

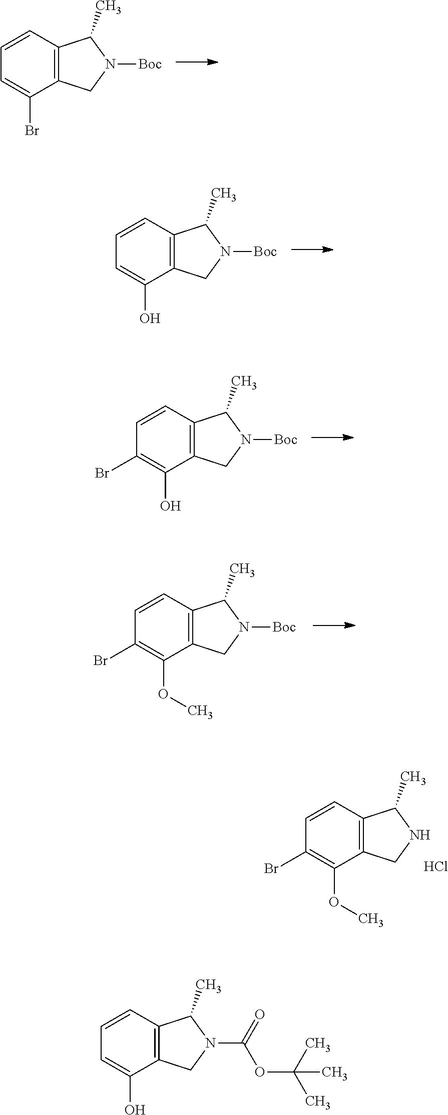

### tert-butyl (1S)-4-hydroxy-1-methyl-isoindoline-2-carboxylate

To a solution of tert-butyl (1S)-4-bromo-1-methyl-isoindoline-2-carboxylate (1.6 g, 5.22 mmol) in DMSO (49 mL) and water (33 mL) were added 2-methylquinolin-8-ol (665 mg, 4.18 mmol), iodocopper (398 mg, 2.09 mmol) and tetrabutylammonium hydroxide (10 mL, 15.7 mmol). The reaction mixture was stirred at 120° C. for 24h then EtOAc was added and the reaction mixture was washed with saturated NaCl solution. The organic layer was dried over Na₂SO₄, filtered and evaporated under reduced pressure. The crude residue was purified by flash chromatography over silica gel column (Cyclohexane/EtOAc) to afford tert-butyl (1S)-4-hydroxy-1-methyl-isoindoline-2-carboxylate (276 mg, 69.1% yield) as a colorless oil. ¹H NMR (400 MHz, DMSO-d6) δ 9.65 (s, 1H), 7.10 (t, J=7.7 Hz, 1H), 6.72 (dd, J=7.5, 2.3 Hz, 1H), 6.68 (d, J=7.9 Hz, 1H), 4.96-4.83 (m, 1H), 4.50 (dd, J=14.9, 8.3 Hz, 1H), 4.38 (td, J=15.5, 2.6 Hz, 1H), 1.46 (s, 9H), 1.40-1.35 (m, 3H).

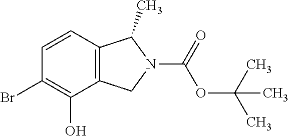 [SMILES: C[C@H]1c2ccc(Br)c(O)c2CN1C(=O)OC(C)(C)C]

### tert-butyl (1S)-5-bromo-4-hydroxy-1-methyl-isoindoline-2-carboxylate

To a solution of tert-butyl (1S)-4-hydroxy-1-methyl-isoindoline-2-carboxylate (276 mg, 1.11 mmol) in DCM (6 mL) were added N-isopropylpropan-2-amine (31 μL, 0.221 mmol) and 1-bromopyrrolidine-2,5-dione (199 mg, 1.11 mmol). The reaction mixture was stirred at ambient temperature for 10 minutes then the solvent was evaporated under reduced pressure. The crude residue was purified by flash chromatography over silica gel column (cyclohexane/EtOAc) to afford tert-butyl (1S)-5-bromo-4-hydroxy-1-methyl-isoindoline-2-carboxylate (292 mg, 80.4% yield). ¹H NMR (400 MHz, DMSO-d6) δ 9.84 (d, J=19.3 Hz, 1H), 7.41 (d, J=8.0 Hz, 1H), 6.72 (d, J=8.1 Hz, 1H), 4.89 (d, J=7.1 Hz, 1H), 4.61 (dd, J=15.7, 4.9 Hz, 1H), 4.47 (t, J=15.9 Hz, 1H), 1.46 (s, 9H), 1.40-1.36 (m, 3H).

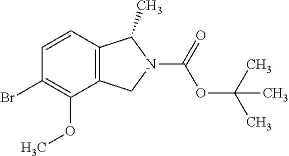 [SMILES: COc1c(Br)ccc2c1CN(C(=O)OC(C)(C)C)[C@H]2C]

### tert-butyl (1S)-5-bromo-4-methoxy-1-methyl-isoindoline-2-carboxylate

To a solution of tert-butyl (1S)-5-bromo-4-hydroxy-1-methyl-isoindoline-2-carboxylate (292 mg, 0.756 mmol) in acetone (3.8 mL) were added dipotassium carbonate (523 mg, 3.78 mmol) and iodomethane (0.14 mL, 2.27 mmol). The reaction mixture was stirred at 50° C. for 1.5 hours then EtOAc was added and the organic layer was washed with saturated NH₄Cl solution then saturated NaCl solution. The organic layer was dried over Na₂SO₄, filtered and evaporated under reduced pressure. The crude residue was purified by flash chromatography over silica gel column (cyclohexane/EtOAc) to afford tert-butyl (1S)-5-bromo-4-methoxy-1-methyl-isoindoline-2-carboxylate (268 mg, 88.0% yield). ¹H NMR (400 MHz, DMSO-d6) δ 7.54 (d, J=8.0 Hz, 1H), 7.01 (t, J=7.1 Hz, 1H), 4.92 (s, 1H), 4.80-4.62 (m, 2H), 3.84 (d, J=3.3 Hz, 3H), 1.47 (s, 9H), 1.40 (s, 3H).

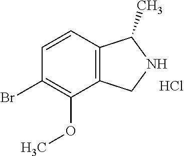

### (1S)-5-bromo-4-methoxy-1-methyl-isoindoline hydrochloride

A solution of tert-butyl (1S)-5-bromo-4-methoxy-1-methyl-isoindoline-2-carboxylate (68 mg, 0.169 mmol) and HCl 4M in dioxane (0.84 mL, 3.38 mmol) was stirred at ambient temperature for 2 hours. The solvent was evaporated under reduced pressure to afford (1S)-5-bromo-4-methoxy-1-methyl-isoindoline hydrochloride (47 mg, 99% yield) as a light yellow solid. ¹H NMR (400 MHz, DMSO-d6) δ 9.62 (s, 2H), 7.65 (d, J=8.1 Hz, 1H), 7.08 (d, J=8.0 Hz, 1H), 4.88 (q, J=6.9 Hz, 12H), 4.72-4.59 (m, 2H), 3.87 (s, 3H), 1.56 (d, J=6.8 Hz, 3H).

### Example 12: Isoindoline Synthesis F

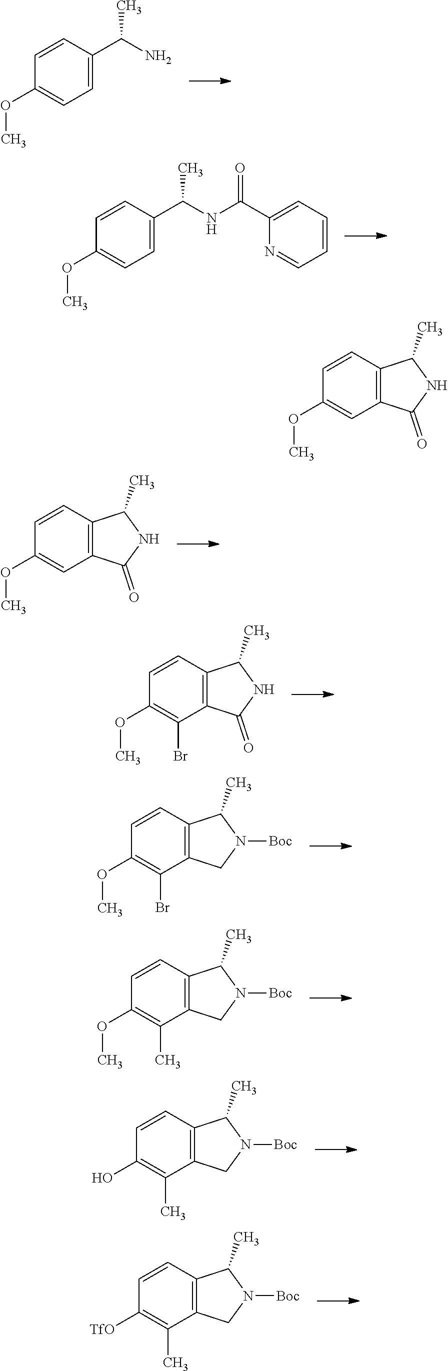

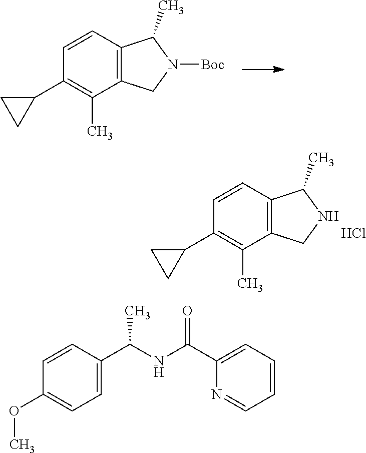

### N-[(1S)-1-(4-methoxyphenyl)ethyl]pyridine-2-carboxamide

A three-necked round bottom flask was successively charged with pyridine-2-carboxylic acid (7.97 g, 62.8 mmol), HATU (26.81 g, 69.1 mmol), DIEA (33 mL, 0.188 mol) and DCM (100 mL). The reaction mixture was cooled down to 0° C. then (1 {S})-1-(4-methoxyphenyl)ethanamine (10.0 g, 62.8 mmol) was added. The reaction mixture was stirred from 0° C. to ambient temperature for 2 hours then water (50 mL) was added and the layers were separated. The organic layer was washed with water (3×50 mL), dried over sodium sulfate, filtered and concentrated under reduced pressure. The crude residue was purified by flash chromatography over silica gel column (CyHex/EtOAc) to afford N-[(1S)-1-(4-methoxyphenyl)ethyl]pyridine-2-carboxamide (15.4 g, 95.7% yield) as a white powder. ¹H NMR (400 MHz, DMSO-d6) δ 8.90 (d, J=8.5 Hz, 1H), 8.65 (dt, J=4.7, 1.3 Hz, 1H), 8.04-7.94 (m, 2H), 7.60 (ddd, J=6.9, 4.7, 1.8 Hz, 1H), 7.39-7.30 (m, 2H), 6.92-6.85 (m, 2H), 5.19-5.06 (m, 1H), 3.72 (s, 3H), 1.50 (d, J=7.0 Hz, 3H).

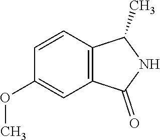

### (3S)-6-methoxy-3-methyl-isoindolin-1-one

In a sealed vial were charged 2,2-dimethylpropanoic acid (12.3 g, 0.120 mol), isopropyl (N{E})-N-isopropoxycarbonyliminocarbamate (24 mL, 0.120 mol), silver carbonate (33.4 g, 0.120 mol), cobalt (2+) acetate hydrate (3.0 g, 12.0 mmol), N-[(1S)-1-(4-methoxyphenyl)ethyl]pyridine-2-carboxamide (15.4 g, 60.1 mmol) and 1-butanol (170 mL). The reaction mixture was stirred at 100° C. for 16 hours then the reaction mixture was filtered over Celite. The filtrate was concentrated under reduced pressure and the crude residue was purified by flash chromatography over silica gel column (DCM/MeOH) to afford (3S)-6-methoxy-3-methyl-isoindolin-1-one (7.5 g, 70.8% yield) as a brown solid. ¹H NMR (400 MHz, DMSO-d6) δ 8.60 (s, 1H), 7.50-7.44 (m, 1H), 7.17-7.10 (m, 2H), 4.59-4.49 (m, 1H), 3.81 (s, 3H), 1.33 (d, J=6.7 Hz, 3H).

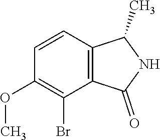

### (3S)-7-bromo-6-methoxy-3-methyl-isoindolin-1-one

To a solution of (3S)-6-methoxy-3-methyl-isoindolin-1-one (2.6 g, 14.7 mmol) in chloroform (75 mL) at ambient temperature under nitrogen was added bromine (0.83 ml, 16.1 mmol). The reaction mixture was stirred for 48 hours then saturated Na₂S₂O₃ solution (20 mL) and EtOAc (20 mL) were added. The layers were separated and the aqueous layer was extracted with EtOAc (3×10 mL). The combined organic layers were dried over sodium sulfate, filtered and concentrated under reduced pressure. The crude residue was purified by flash chromatography over silica gel column (CyHex/EtOAc) to afford (3S)-7-bromo-6-methoxy-3-methyl-isoindolin-1-one (2.06 g, 54.8% yield) as a light yellow solid. ¹H NMR (400 MHz, DMSO-d6) δ 8.72 (s, 1H), 7.52 (dd, J=8.2, 0.8 Hz, 1H), 7.32 (d, J=8.3 Hz, 1H), 4.54-4.45 (m, 1H), 3.88 (s, 3H), 1.40 (s, 2H), 1.33 (d, J=6.6 Hz, 3H).

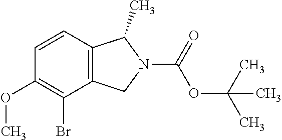 [SMILES: COc1ccc2c(c1Br)CN(C(=O)OC(C)(C)C)[C@H]2C]

### tert-butyl (1S)-4-bromo-5-methoxy-1-methyl-isoindoline-2-carboxylate

To a solution of (3S)-7-bromo-6-methoxy-3-methyl-isoindolin-1-one (2.1 g, 8.04 mmol) in THF (40 mL) at ambient temperature under nitrogen was added sodium borohydride (1.54 g, 40.2 mmol). The reaction mixture was cooled down to 0° C. and ethoxyethane trifluoroborane (6.1 mL, 48.3 mmol) was added. The reaction mixture was stirred to reflux for 4 hours then cooled down to 0° C. and 1M aqueous HCl (15 mL) then EtOAc were added. The layers were separated and the aqueous layer was washed 2 times with EtOAc then basified by the addition of 30% aqueous NaOH and extracted with EtOAc. The organic layer was washed with saturated NaCl solution, dried over Na₂SO₄ and concentrated under reduced pressure to afford (1S)-4-bromo-5-methoxy-1-methyl-isoindoline. To a solution of the residue in THF (40 mL) at ambient temperature under nitrogen were successively added tert-butoxycarbonyl tert-butyl carbonate (1.8 g, 8.04 mmol) and N-ethyl-N-isopropyl-propan-2-amine (1.4 mL, 8.04 mmol). The reaction mixture was stirred at ambient temperature for 16 hours then saturated NaHCO₃ solution was added. The layers were separated and the organic layer was washed with saturated NaCl solution, dried over Na₂SO₄, concentrated under reduced pressure and concentrated under reduced pressure. The crude residue was purified by flash chromatography over silica gel column (CyHex/EtOAc) to afford tert-butyl (1S)-4-bromo-5-methoxy-1-methyl-isoindoline-2-carboxylate (2.37 g, 86.1% yield) as a pink solid. ¹H NMR (400 MHz, DMSO-d6) δ 7.27 (dd, J=8.1, 2.3 Hz, 1H), 7.04 (d, J=8.3 Hz, 1H), 5.01 (t, J=6.9 Hz, 1H), 4.59-4.36 (m, 2H), 3.84 (s, 3H), 1.46 (s, 9H), 1.43-1.37 (m, 3H).

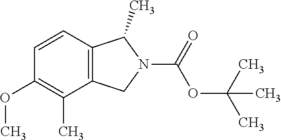 [SMILES: COc1ccc2c(c1C)CN(C(=O)OC(C)(C)C)[C@H]2C]

### tert-butyl (1S)-5-methoxy-1,4-dimethyl-isoindoline-2-carboxylate

To a solution of tert-butyl (1S)-4-bromo-5-methoxy-1-methyl-isoindoline-2-carboxylate (500 mg, 1.46 mmol) in THF (7 mL) at ambient temperature under nitrogen were successively added tri-tert-butylphosphonium tetrafluoroborate (26 mg, 0.088 mmol), dichlorozinc (0.5M in THF, 0.88 mL, 0.438 mmol) and diacetoxypalladium (16 mg, 0.073 mmol) then bromo(methyl)magnesium (3M in THF, 0.97 mL, 2.92 mmol) was added at 0° C. The reaction mixture was stirred at ambient temperature for 2 hours then the reaction mixture was filtered and saturated NH₄Cl solution (25 mL) and EtOAc (25 mL) were added. The layers were separated and the aqueous layer was extracted with EtOAc (3×10 mL). The combined organic layers were dried over sodium sulfate, filtered and concentrated under reduced pressure. The crude residue was purified by flash chromatography over silica gel column (CyHex/EtOAc) to afford tert-butyl (1S)-5-methoxy-1,4-dimethyl-isoindoline-2-carboxylate (436 mg, 99% yield) as a yellow oil. ¹H NMR (400 MHz, DMSO-d6) δ 7.06 (d, J=8.4 Hz, 1H), 6.88 (d, J=8.3 Hz, 1H), 4.89 (s, 1H), 4.58-4.36 (m, 2H), 3.77 (s, 3H), 2.04 (s, 3H), 1.46 (s, 9H), 1.38 (d, J=6.1 Hz, 3H).

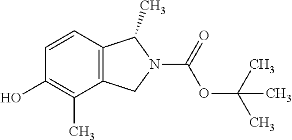 [SMILES: Cc1c(O)ccc2c1CN(C(=O)OC(C)(C)C)[C@H]2C]

### tert-butyl (1S)-5-hydroxy-1,4-dimethyl-isoindoline-2-carboxylate

To a solution of tert-butyl (1S)-5-methoxy-1,4-dimethyl-isoindoline-2-carboxylate (438 mg, 1.58 mmol) in DMF (5 mL) at ambient temperature under nitrogen was added sodium thiomethanolate (455 mg, 6.32 mmol). The reaction mixture was stirred at 140° C. for 1 hour then saturated NH₄Cl solution (20 mL) and EtOAc (20 mL) were added. The layers were separated and the aqueous layer was extracted with EtOAc (2×20 mL). The combined organic layers were dried over sodium sulfate, filtered and concentrated under reduced pressure. The crude residue was purified by flash chromatography over silica gel column (CyHex/EtOAc) to afford tert-butyl (1S)-5-hydroxy-1,4-dimethyl-isoindoline-2-carboxylate (407 mg, 96.9% yield) as a light beige solid. ¹H NMR (400 MHz, DMSO-d6) δ 9.21 (s, 1H), 6.91-6.84 (m, 1H), 6.72 (d, J=8.1 Hz, 1H), 4.84 (t, J=6.7 Hz, 1H), 4.55-4.32 (m, 2H), 2.01 (d, J=2.4 Hz, 3H), 1.45 (s, 9H), 1.35 (dd, J=6.4, 3.1 Hz, 3H).

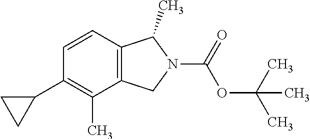 [SMILES: Cc1c(C2CC2)ccc2c1CN(C(=O)OC(C)(C)C)[C@H]2C]

### tert-butyl (1S)-5-cyclopropyl-1,4-dimethyl-isoindoline-2-carboxylate

To a solution of tert-butyl (1S)-5-hydroxy-1,4-dimethyl-isoindoline-2-carboxylate (20 mg, 0.076 mmol) in DCM (0.5 mL) at 0° C. were added pyridine (0.012 mL, 0.152 mmol) and trifluoromethylsulfonyl trifluoromethanesulfonate (0.012 mL, 0.072 mmol). The reaction mixture was stirred at ambient temperature for 4 hours then the solvent was evaporated under reduced pressure to afford tert-butyl (1S)-5-bromo-1-methyl-4-(trifluoromethylsulfonyloxy)isoindoline-2-carboxylate. The compound was used as such in the next step.

To a solution of crude tert-butyl (1S)-1,4-dimethyl-5-(trifluoromethylsulfonyloxy)isoindoline-2-carboxylate (225 mg, 0.569 mmol) in toluene (5 mL) under nitrogen were added cyclopropyl boronic acid (102 mg, 1.14 mmol) and tripotassium phosphate (362 mg, 1.71 mmol). The reaction mixture was stirred at ambient temperature under nitrogen for 5 minutes then cyclopentyl(diphenyl)phosphane dichloropalladium iron (88 mg, 0.114 mmol) was added and the reaction mixture was stirred at 140° C. for 1 hour under micro-wave irradiation. The reaction mixture was diluted with EtOAc (10 mL) and water (10 mL) was added. The layers were separated and the organic layer was dried over sodium sulfate, filtered and concentrated under reduced pressure. The crude residue was purified by flash chromatography over silica gel column (CyHex/EtOAc) to afford tert-butyl (1S)-5-cyclopropyl-1,4-dimethyl-isoindoline-2-carboxylate (50 mg, 30.6% yield) as a yellow oil. ¹H NMR (400 MHz, DMSO-d6) δ 7.00 (d, J=8.2 Hz, 1H), 6.91 (d, J=7.9 Hz, 1H), 4.90 (d, J=6.6 Hz, 1H), 4.62-4.40 (m, 2H), 2.26 (d, J=2.5 Hz, 3H), 1.89 (tt, J=8.5, 5.4 Hz, 1H), 1.46 (d, J=1.7 Hz, 9H), 1.40 (s, 3H), 0.95-0.85 (m, 2H), 0.55 (td, J=5.8, 3.9 Hz, 2H).

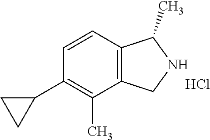

### (1S)-5-cyclopropyl-1,4-dimethyl-isoindoline Hydrochloride

To a solution of tert-butyl (1S)-5-cyclopropyl-1,4-dimethyl-isoindoline-2-carboxylate (52 mg, 0.181 mmol) in DCM (0.9 mL) was added 4M hydrogen chloride in dioxane (0.90 mL, 3.62 mmol). The reaction mixture was stirred at ambient temperature for 1 hour then concentrated under reduced pressure. The crude residue (1S)-5-cyclopropyl-1,4-dimethyl-isoindoline hydrochloride (42 mg, 99% yield), brown gum was used without further purification. ¹H NMR (400 MHz, DMSO-d6) δ 10.01 (s, 1H), 9.45 (s, 1H), 7.08 (d, J=7.9 Hz, 1H), 6.99 (d, J=7.9 Hz, 1H), 4.87 (dq, J=12.5, 6.3 Hz, 1H), 4.55-4.36 (m, 2H), 2.31 (s, 3H), 1.92 (ddd, J=13.8, 8.6, 5.4 Hz, 1H), 1.54 (d, J=6.8 Hz, 3H), 0.99-0.87 (m, 2H), 0.58 (td, J 5.6, 3.6 Hz, 2H).

### Example 12: Isoindoline Synthesis G

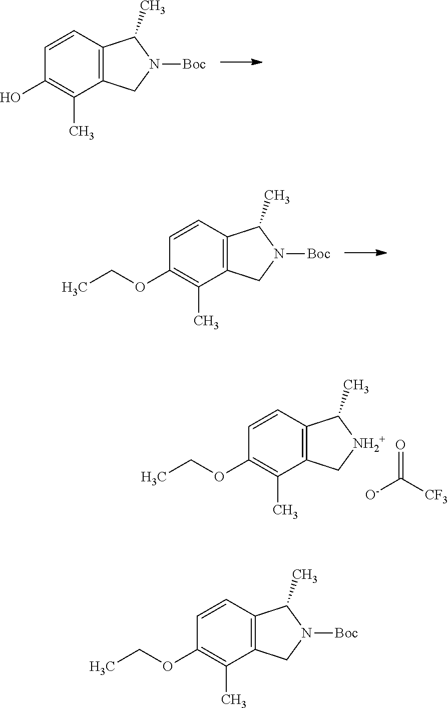 [SMILES: Cc1c(O)ccc2c1CN(C(=O)OC(C)(C)C)[C@H]2C ; CCOc1ccc2c(c1C)CN(C(=O)OC(C)(C)C)[C@H]2C ; CCOc1ccc2c(c1C)C[NH2+][C@H]2C ; O=C([O-])C(F)(F)F ; CCOc1ccc2c(c1C)CN(C(=O)OC(C)(C)C)[C@H]2C]

### tert-butyl (1S)-5-hydroxy-1,4-dimethyl-isoindoline-2-carboxylate

To a solution of tert-butyl (1S)-5-hydroxy-1,4-dimethyl-isoindoline-2-carboxylate (100 mg, 0.38 mmol) in acetonitrile (2 mL) were added cesium carbonate (373 mg, 1.14 mmol) and iodoethane (0.061 mL, 0.76 mmol). The reaction mixture was stirred under reflux for 1 hour then EtOAc and saturated NaHCO₃ solution were added. The layers were separated. The organic layer was dried over Na₂SO₄, filtered and concentrated under reduced pressure to afford tert-butyl (1S)-5-hydroxy-1,4-dimethyl-isoindoline-2-carboxylate (92 mg, 99% yield) as a colorless oil. ¹H NMR (400 MHz, DMSO-d6) δ 7.04 (d, J=8.2 Hz, 1H), 6.87 (d, J=8.3 Hz, 1H), 4.89 (d, J=6.9 Hz, 1H), 4.58-4.38 (m, 2H), 4.01 (q, J=7.0 Hz, 2H), 2.05 (s, 3H), 1.46 (s, 9H), 1.38 (dd, J=6.3, 3.0 Hz, 3H), 1.33 (t, J=6.9 Hz, 3H).

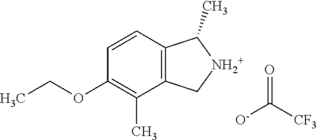

### (1S)-5-ethoxy-1,4-dimethyl-2,3-dihydro-1H-isoindol-2-ium trifluoroacetate

To a solution of tert-butyl (1S)-5-ethoxy-1,4-dimethyl-isoindoline-2-carboxylate (92 mg, 0.30 mmol) in DCM (2 mL) at 0° C. was added 2,2,2-trifluoroacetic acid 20% in DCM (0.46 mL, 1.21 mmol). The reaction mixture was stirred at ambient temperature for 2 hours then the solvent was evaporated under reduced pressure to afford (1S)-5-ethoxy-1,4-dimethyl-2,3-dihydro-1H-isoindol-2-ium trifluoroacetate (92 mg, 80% yield) which was used without further purification. ¹H NMR (400 MHz, DMSO-d6) δ 7.04 (d, J=8.2 Hz, 1H), 6.87 (d, J=8.3 Hz, 1H), 4.89 (d, J=6.9 Hz, 1H), 4.58-4.38 (m, 2H), 4.01 (q, J=7.0 Hz, 2H), 2.05 (s, 3H), 1.46 (s, 9H), 1.38 (dd, J=6.3, 3.0 Hz, 3H), 1.33 (t, J=6.9 Hz, 3H).

### Example 13: Isoindoline Synthesis H

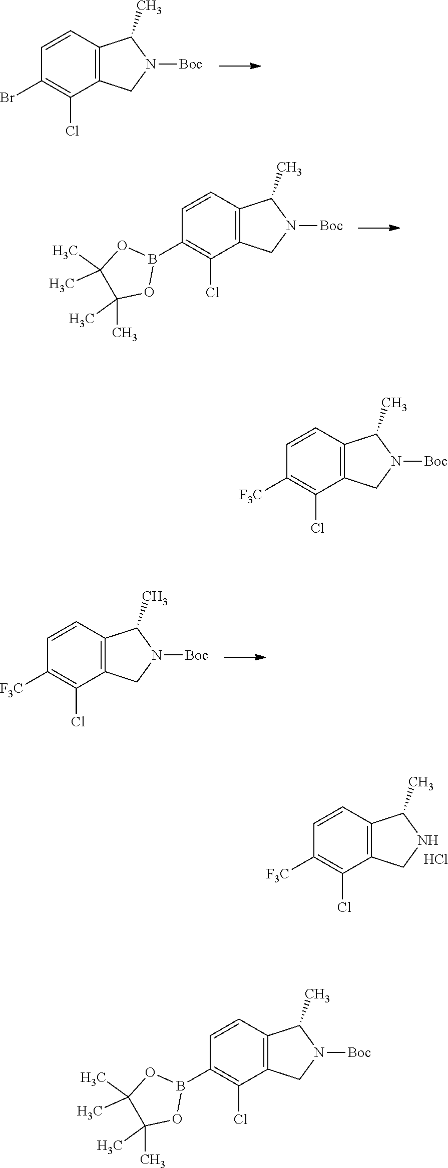

### tert-butyl (1S)-4-chloro-1-methyl-5-(4,4,5,5-tetramethyl-1,3,2-dioxaborolan-2-yl)isoindoline-2-carboxylate

To a solution of tert-butyl (1S)-5-bromo-4-chloro-1-methyl-isoindoline-2-carboxylate (258 mg, 0.744 mmol) in DMF (2 mL) were added 4,4,5,5-tetramethyl-2-(4,4,5,5-tetramethyl-1,3,2-dioxaborolan-2-yl)-1,3,2-dioxaborolane (286 mg, 1.12 mmol), cyclopentyl(diphenyl)phosphane: dichloromethane: dichloropalladium iron (19 mg, 0.022 mmol) and potassium acetate (219 mg, 2.23 mmol). The reaction mixture was stirred at 80° C. for 16 hours then water (10 mL) was added. The layers were separated and the aqueous layer was extracted with iPrOAc (2×10 mL). The combined organic layers were dried through a layer separator and evaporated under reduced pressure. The crude residue was purified by flash chromatography over silica gel column (Hept/EtOAc) to afford tert-butyl (1S)-4-chloro-1-methyl-5-(4,4,5,5-tetramethyl-1,3,2-dioxaborolan-2-yl)isoindoline-2-carboxylate (281 mg, 95.9% yield) as a pink oil. 1H NMR (400 MHz, DMSO-d6): δ (ppm) 7.64-7.58 (m, 1H), 7.32-7.26 (m, 1H), 5.06 (s, 1H), 4.66-4.44 (m, 2H), 1.47 (s, 9H), 1.43 (dd, J=6.5, 2.4 Hz, 4H), 1.31 (s, 11H).

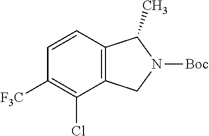 [SMILES: C[C@H]1c2ccc(C(F)(F)F)c(Cl)c2CN1C(=O)OC(C)(C)C]

### tert-butyl (1S)-4-chloro-1-methyl-5-(trifluoromethyl)isoindoline-2-carboxylate

To a solution of copper(1+) trifluoromethanesulfonate benzene (2:2:1) (77 mg, 0.152 mmol), 1,10-phenanthroline (55 mg, 0.305 mmol), tripotassium phosphate (164 mg, 0.762 mmol), potassium fluoride (75 mg, 1.27 mmol) and silver carbonate (70 mg, 0.254 mmol) in DMF (3 mL) was added trimethyl(trifluoromethyl)silane (181 mg, 1.27 mmol). The reaction mixture was stirred at 45° C. for 20 minutes then a solution of tert-butyl (1S)-4-chloro-1-methyl-5-(4,4,5,5-tetramethyl-1,3,2-dioxaborolan-2-yl)isoindoline-2-carboxylate (100 mg, 0.254 mmol) in DMF (2 mL) was added. The reaction mixture was stirred at 50° C. for 16 hours then water (10 mL) was added and the layers were separated. The aqueous layer was extracted with iPrOAc (2×10 mL). The combined organic layers were dried through a layer separator and concentrated under reduced pressure. The crude residue was purified by flash chromatography over silica gel column (Hept/EtOAc) to afford tert-butyl (1S)-4-chloro-1-methyl-5-(trifluoromethyl)isoindoline-2-carboxylate (9 mg, 10.5% yield) as a colorless oil. ¹H NMR (400 MHz, DMSO-d6): δ (ppm) 7.83 (d, J=8.0 Hz, 1H), 7.52 (d, J=7.9 Hz, 1H), 5.14 (s, 1H), 4.81-4.51 (m, 3H), 1.47 (s, 14H).

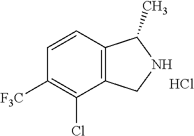

### (1S)-4-chloro-1-methyl-5-(trifluoromethyl)isoindoline Hydrochloride

To a solution of tert-butyl (1S)-4-chloro-1-methyl-5-(trifluoromethyl)isoindoline-2-carboxylate (32 mg, 0.095 mmol) in DCM (1 mL) was added hydrogen chloride 4N in dioxane (0.24 mL, 0.953 mmol). The reaction mixture was stirred at ambient temperature for 1 hour then concentrated under reduced pressure to afford intermediate (1S)-4-chloro-1-methyl-5-(trifluoromethyl)isoindoline hydrochloride (25 mg, 96.4% yield) which was engaged in the next step without further purification.

### Example 14: Isoindoline Synthesis I

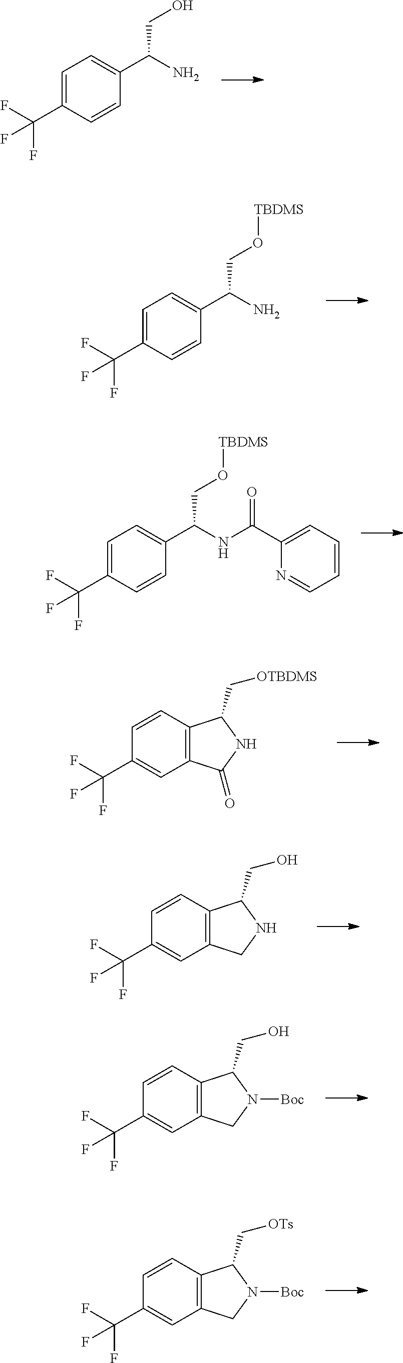

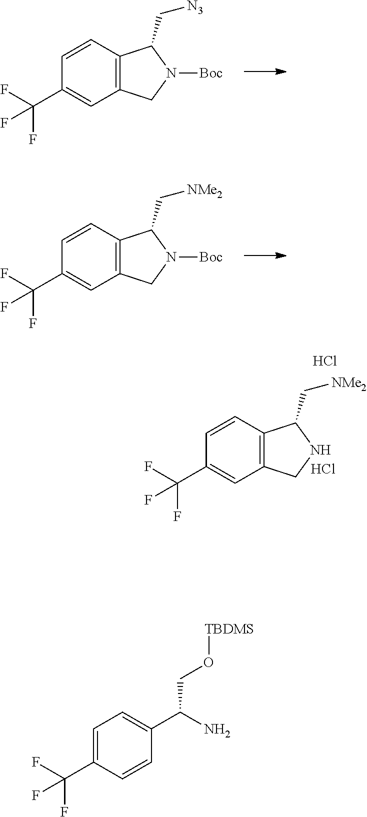

### (1R)-2-[tert-butyl(dimethyl)silyl]oxy-1-[4-(trifluoromethyl)phenyl]ethanamine

To a solution of (2R)-2-amino-2-[4-(trifluoromethyl)phenyl]ethanol (10.0 g, 48.7 mmol) in acetonitrile (80 mL) at ambient temperature under nitrogen were successively added tert-butyl-chloro-dimethyl-silane (10.7 g, 69.9 mmol) and 2,3,4,6,7,8,9,10-octahydropyrimido[1,2-a]azepine (11 mL, 71.5 mmol). The reaction mixture was stirred at ambient temperature for 3 hours then poured into EtOAc (200 mL). The suspension was filtered and the filtrate was concentrated under reduced pressure to afford (1R)-2-[tert-butyl(dimethyl)silyl]oxy-1-[4-(trifluoromethyl)phenyl]ethanamine (18 g, 99.9% yield) as a colorless oil. ¹H NMR (400 MHz, DMSO-d6): δ (ppm) 7.69-7.53 (m, 4H), 3.98 (dt, J=13.7, 6.1 Hz, 1H), 3.69-3.43 (m, 2H), 0.83-0.73 (m, 9H), −0.03-0.12 (m, 6H).

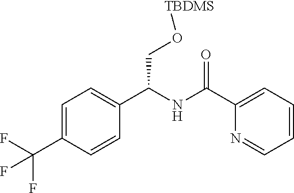

### N-[(1R)-2-[tert-butyl(dimethyl)silyl]oxy-1-[4-(trifluoromethyl)phenyl]ethyl]pyridine-2-carboxamide

To a solution of (1R)-2-[tert-butyl(dimethyl)silyl]oxy-1-[4-(trifluoromethyl)phenyl]ethanamine (16.20 g, 50.7 mmol) in DCM (80 mL) at ambient temperature under nitrogen were successively added pyridine-2-carboxylic acid (7.72 g, 60.9 mmol), [dimethylamino(triazolo[4,5-b]pyridin-3-yloxy)methylene]-dimethyl-ammonium; hexafluorophosphate (23.6 g, 60.9 mmol) and N-ethyl-N-isopropyl-propan-2-amine (31 mL, 0.177 mol). The reaction mixture was stirred at ambient temperature for 2 hours then saturated NH₄Cl solution (100 mL) and iPrOAc (100 mL) were added. The layers were separated and the aqueous layer was extracted with iPrOAc (3×50 mL). The combined organic layers were dried over sodium sulfate, filtered and concentrated under reduced pressure. The crude residue was purified by flash chromatography over silica gel column (DCM/MeOH) to afford N-[(1R)-2-[tert-butyl(dimethyl)silyl]oxy-1-[4-(trifluoromethyl)phenyl]ethyl]pyridine-2-carboxamide (16.6 g, 77.1% yield) as a yellow oil. ¹H NMR (400 MHz, DMSO-d6): δ (ppm) 9.16 (d, J=8.5 Hz, 1H), 8.69 (dt, J=4.7, 1.4 Hz, 1H), 8.06-7.97 (m, 2H), 7.70 (d, J=8.4 Hz, 2H), 7.70-7.59 (m, 3H), 5.21 (dt, J=8.5, 5.6 Hz, 1H), 4.07-3.93 (m, 2H), 0.80 (s, 9H), −0.04 (d, J=15.1 Hz, 6H).

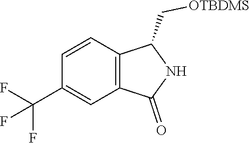 [SMILES: CC(C)(C)[Si](C)(C)OC[C@@H]1NC(=O)c2cc(C(F)(F)F)ccc21]

### (3R)-3-{[(tert-butyldimethylsilyl)oxy]methyl}-6-(trifluoromethyl)-2,3-dihydro-1H-isoindol-1-one

An autoclave was charged with N-[(1R)-2-[tert-butyl(dimethyl)silyl]oxy-1-[4-(trifluoromethyl)phenyl]ethyl]pyridine-2-carboxamide (16.60 g, 39.1 mmol), 2,2-dimethylpropanoic acid (7.99 g, 78.2 mmol), isopropyl (N{E})-N-isopropoxycarbonyliminocarbamate (15 mL, 78.2 mmol), silver carbonate (21.72 g, 78.2 mmol), cobalt(2+) acetate hydrate (1:2:4) (1.95 g, 7.82 mmol) and 2,2,2-trifluoroethanol (120 mL). The reaction mixture was stirred at 100° C. for 16 hours then cooled down to ambient temperature and filtered over dicalite. The filtrate was concentrated under reduced pressure. The crude residue was dissolved in IPrOAc (200 mL) and saturated NaHCO₃ solution (200 mL) was added. The layers were separated and the organic layer was washed with saturated NaHCO₃ solution (200 mL). The combined organic layers were washed with saturated NaCl solution (200 mL), dried over MgSO₄ and concentrated under reduced pressure. The crude residue was purified by flash purification over silica gel column to afford (3R)-3-{[(tert-butyldimethylsilyl)oxy]methyl}-6-(trifluoromethyl)-2,3-dihydro-1H-isoindol-1-one (8.6 g, 52.6% yield) as an orange solid. ¹H NMR (400 MHz, DMSO-d6) δ 8.84 (s, 1H), 7.97 (ddd, J=8.0, 1.8, 0.7 Hz, 1H), 7.90-7.82 (m, 2H), 4.74 (t, J=4.6 Hz, 1H), 3.95 (dd, J=10.3, 4.4 Hz, 1H), 3.80 (dd, J=10.3, 4.9 Hz, 1H), 0.72 (s, 9H), −0.01 (s, 3H), −0.06 (s, 3H).

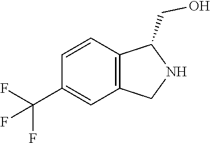 [SMILES: OC[C@@H]1NCc2cc(C(F)(F)F)ccc21]

### [(1R)-5-(trifluoromethyl)isoindolin-1-yl]methanol

To a solution of (3R)-3-[[tert-butyl(dimethyl)silyl]oxymethyl]-6-(trifluoromethyl)isoindolin-1-one (8.66 g, 20.6 mmol) in THF (90 mL) at 0° C. were added lithium aluminium hydride (2M in THF, 51 mL, 0.103 mol) and chloro(trimethyl)silane (7.8 mL, 61.7 mmol). The reaction mixture was stirred at ambient temperature for 16 hours. The reaction mixture was stopped using the Fieser quench to afford [(1R)-5-(trifluoromethyl)isoindolin-1-yl]methanol (4.09 g, 91.6% yield) as a red oil which was used without further purification in the next step. ¹H NMR (400 MHz, DMSO-d6) δ 7.61-7.58 (m, 1H), 7.55-7.52 (m, 2H), 4.76 (dt, J=12.4, 6.1 Hz, 1H), 4.34 (d, J=6.2 Hz, 1H), 4.14 (dt, J=5.0, 1.5 Hz, 2H), 3.53 (dd, J=5.8, 3.1 Hz, 2H), 3.38 (td, J=6.7, 3.0 Hz, 1H).

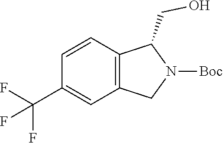 [SMILES: CC(C)(C)OC(=O)N1Cc2cc(C(F)(F)F)ccc2[C@@H]1CO]

### tert-butyl (1R)-1-(hydroxymethyl)-5-(trifluoromethyl)isoindoline-2-carboxylate

To a solution of [(1R)-5-(trifluoromethyl)isoindolin-1-yl]methanol (4.1 g, 18.8 mmol) in DCM (40 mL) under nitrogen were added tert-butoxycarbonyl tert-butyl carbonate (4.9 g, 22.6 mmol) and N-ethyl-N-isopropyl-propan-2-amine (4.9 mL, 28.2 mmol). The reaction mixture was stirred at ambient temperature for 1 hour then concentrated under reduced pressure. The crude residue was dissolved in EtOAc and washed with saturated NH₄Cl solution, dried over sodium sulfate, filtered and concentrated under reduced pressure. The crude residue was purified by flash chromatography over silica gel column (CyHex/EtOAc) to afford tert-butyl (1R)-1-(hydroxymethyl)-5-(trifluoromethyl)isoindoline-2-carboxylate (2.61 g, 43.7% yield) as a red oil. ¹H NMR (400 MHz, DMSO-d6) δ 7.70 (s, 1H), 7.67-7.61 (m, 1H), 7.58 (t, J=6.6 Hz, 1H), 4.95 (d, J=14.3 Hz, 1H), 4.86-4.75 (m, 1H), 4.71 (dd, J=15.4, 3.6 Hz, 1H), 4.54 (t, J=15.8 Hz, 1H), 3.82 (dt, J=11.0, 3.6 Hz, 1H), 3.74 (dt, J=10.2, 4.6 Hz, 1H), 1.46 (s, 9H).

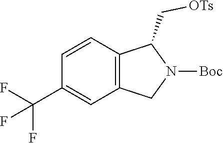 [SMILES: Cc1ccc(S(=O)(=O)OC[C@H]2c3ccc(C(F)(F)F)cc3CN2C(=O)OC(C)(C)C)cc1]

### (tert-butyl (1R)-1-(p-tolylsulfonyloxymethyl)-5-(trifluoromethyl)isoindoline-2-carboxylate

To a solution of tert-butyl (1R)-1-(hydroxymethyl)-5-(trifluoromethyl)isoindoline-2-carboxylate (498 mg, 1.44 mmol) in DCM (5 mL) were added triethylamine (0.52 mL, 3.73 mmol) and DMAP (15 mg, 0.124 mmol). The mixture was cooled down to 0° C. 4-methylbenzenesulfonyl chloride (356 mg, 1.87 mmol) was then added and the reaction mixture was stirred at ambient temperature for 2 hours. Saturated NaHCO₃ solution was added, the layers were separated and the organic layer was washed with saturated NaCl solution, dried over MgSO₄ and concentrated under reduced pressure. The crude residue was purified by flash chromatography over silica gel column (CyHex/EtOAc) to afford (tert-butyl (1R)-1-(p-tolylsulfonyloxymethyl)-5-(trifluoromethyl)isoindoline-2-carboxylate (535 mg, 78.6% yield) as a light purple oil. ¹H NMR (400 MHz, DMSO-d6) δ 7.76-7.70 (m, 1H), 7.64-7.54 (m, 3H), 7.47 (t, J=6.8 Hz, 1H), 7.40 (t, J=7.4 Hz, 2H), 5.21 (s, 1H), 4.67 (d, J=15.3 Hz, 1H), 4.59-4.38 (m, 3H), 2.40 (s, 3H), 1.38 (d, J=25.3 Hz, 10H).

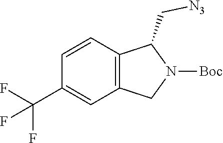 [SMILES: CC(C)(C)OC(=O)N1Cc2cc(C(F)(F)F)ccc2[C@@H]1CN=[N+]=[N-]]

### tert-butyl (1R)-1-(azidomethyl)-5-(trifluoromethyl)isoindoline-2-carboxylate

To a solution of tert-butyl (1R)-1-(p-tolylsulfonyloxymethyl)-5-(trifluoromethyl)isoindoline-2-carboxylate (387 mg, 0.821 mmol) in DMF (20 mL) was added sodium azide (133 mg, 2.05 mmol). The reaction mixture was stirred under argon for 1.5 hour at 80° C. then EtOAc and water were added. The layers were separated and the organic layer was extracted with water. The combined organic layers were washed with saturated NaCl solution, dried over magnesium sulfate, filtered and concentrated under reduced pressure to afford tert-butyl (1R)-1-(azidomethyl)-5-(trifluoromethyl)isoindoline-2-carboxylate (224 mg, 74.9% yield) as a light yellow oil. ¹H NMR (400 MHz, DMSO-d6) δ 7.78 (s, 1H), 7.74-7.68 (m, 1H), 7.63 (d, J=8.2 Hz, 1H), 5.26 (s, 1H), 4.77 (d, J=15.4 Hz, 1H), 4.60 (dd, J=21.8, 15.4 Hz, 1H), 4.06 (ddd, J=49.9, 12.9, 3.9 Hz, 1H), 3.79-3.60 (m, 1H), 1.48 (d, J=3.8 Hz, 9H).

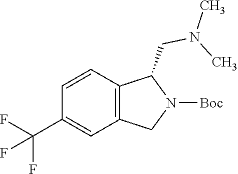 [SMILES: CN(C)C[C@H]1c2ccc(C(F)(F)F)cc2CN1C(=O)OC(C)(C)C]

### (tert-butyl (1R)-1-[(dimethylamino)methyl]-5-(trifluoromethyl)isoindoline-2-carboxylate

To a solution of tert-butyl (1R)-1-(azidomethyl)-5-(trifluoromethyl)isoindoline-2-carboxylate (47 mg, 0.129 mmol) in methanol (1 mL) was added formaldehyde (37% in water, 51 μL, 0.643 mmol). The reaction mixture was degassed with argon and palladium (10% on charcoal, 14 mg, 0.013 mmol) was added. The reaction mixture was stirred under a hydrogen atmosphere (0.3 bar) for 20 h at ambient temperature. The reaction mixture was then degassed with argon, filtered, washed with methanol and concentrated under reduced pressure. The crude residue was taken up in DCM and dried over magnesium sulfate, filtered and concentrated under reduced pressure. The crude residue was purified by flash chromatography over silica gel column (CyHex/EtOAc) to afford (tert-butyl (1R)-1-[(dimethylamino)methyl]-5-(trifluoromethyl)isoindoline-2-carboxylate (32 mg, 67.3% yield) as a colorless oil. ¹H NMR (400 MHz, DMSO-d6) δ 7.71 (s, 1H), 7.63 (d, J=1.9 Hz, 2H), 5.06-4.90 (m, 1H), 4.72 (d, J=15.4 Hz, 1H), 4.54 (t, J=17.0 Hz, 1H), 2.69 (t, J=14.3 Hz, 1H), 2.56-2.42 (m, 1H), 2.18 (d, J=9.5 Hz, 6H), 1.46 (d, J=3.7 Hz, 9H).

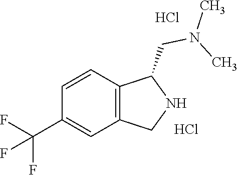 [SMILES: CN(C)C[C@@H]1NCc2cc(C(F)(F)F)ccc21 ; Cl ; Cl]

### N,N-dimethyl-1-[(1R)-5-(trifluoromethyl)isoindolin-1-yl]methanamine dihydrochloride

A solution of tert-butyl (1R)-1-[(dimethylamino)methyl]-5-(trifluoromethyl)isoindoline-2-carboxylate (38 mg, 0.109 mmol) and 4M hydrogen chloride in dioxane (0.60 mL, 2.40 mmol) was stirred at ambient temperature for 1.5 hour. The reaction mixture was concentrated under reduced pressure to afford N,N-dimethyl-1-[(1R)-5-(trifluoromethyl)isoindolin-1-yl]methanamine hydrochloride (34 mg, 99.5% yield) as a purple oil. The crude residue was used in the next step without further purification. ¹H NMR (400 MHz, DMSO-d6) δ 7.91-7.82 (m, 2H), 7.75 (d, J=8.0 Hz, 1H), 5.56 (d, J=9.2 Hz, 1H), 4.74 (d, J=15.1 Hz, 1H), 4.64 (d, J=15.1 Hz, 1H), 3.92-3.69 (m, 2H), 2.96 (s, 6H).

### Example 15: Isoindoline Synthesis J

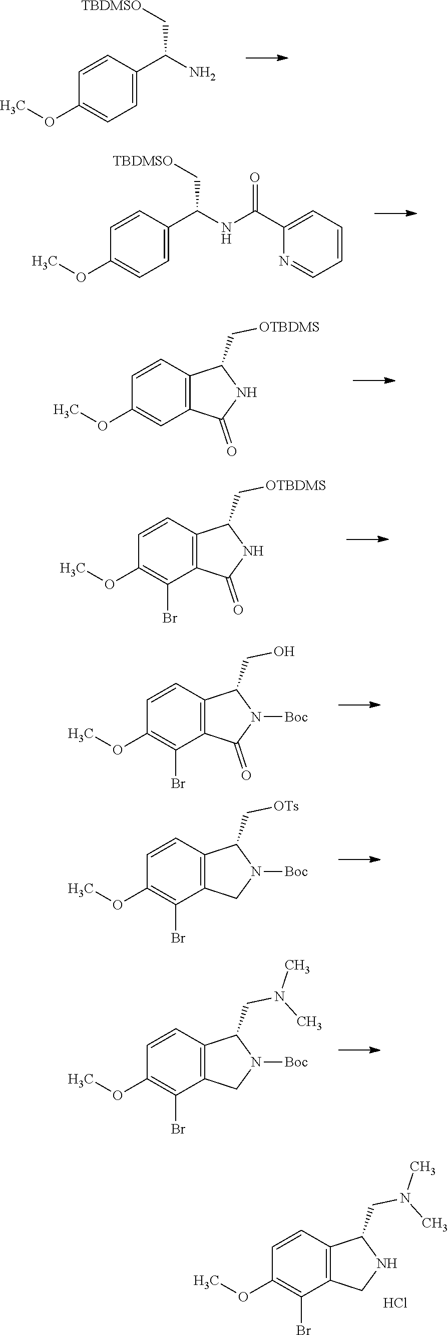

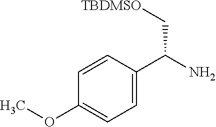 [SMILES: COc1ccc([C@@H](N)CO[Si](C)(C)C(C)(C)C)cc1]

### (1R)-2-[tert-butyl(dimethyl)silyl]oxy-1-(4-methoxyphenyl)ethanamine

To a solution of (2R)-2-amino-2-(4-methoxyphenyl)ethanol (10.0 g, 59.8 mmol) in acetonitrile (100 mL) at ambient temperature under nitrogen were successively added tert-butyl-chloro-dimethyl-silane (13.8 g, 89.7 mmol) and 2,3,4,6,7,8,9,10-octahydropyrimido[1,2-a]azepine (14 mL, 89.7 mmol). The reaction mixture was stirred at ambient temperature for 1 hour then poured into EtOAc (100 mL). The organic layer was washed with saturated NaHCO₃ solution (100 mL), dried over sodium sulfate, filtered and concentrated under reduced pressure to afford (1R)-2-[tert-butyl(dimethyl)silyl]oxy-1-(4-methoxyphenyl)ethanamine (16.8 g, 99.8% yield) as a colorless oil. ¹H NMR (400 MHz, DMSO-d6) δ 7.31-7.16 (m, 2H), 6.91-6.76 (m, 2H), 3.87-3.75 (m, 1H), 3.72 (d, J=1.7 Hz, 2H), 3.56-3.38 (m, 2H), 0.86-0.78 (m, 9H), −0.01-0.10 (m, 6H).

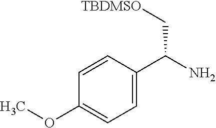 [SMILES: COc1ccc([C@@H](N)CO[Si](C)(C)C(C)(C)C)cc1]

### N-[(1R)-2-[tert-butyl(dimethyl)silyl]oxy-1-(4-methoxyphenyl)ethyl]pyridine-2-carboxamide

To a solution of pyridine-2-carboxylic acid (9.0 g, 71.6 mmol) in DCM (95 mL) at 0° C. under nitrogen were successively added N-ethyl-N-isopropyl-propan-2-amine (36.48 mL, 208.9 mmol) and 2,4,6-tripropyl-1,3,5,2{5},4{5},6{5}-trioxatriphosphinane 2,4,6-trioxide (42.64 mL, 71.6 mmol) The reaction mixture was stirred at 0° C. for 5 minutes and (1R)-2-[tert-butyl(dimethyl)silyl]oxy-1-(4-methoxyphenyl)ethanamine (16.8 g, 59.7 mmol) was added. The reaction mixture was stirred from 0° C. to ambient temperature for 1 hour then saturated NaHCO₃ solution (100 mL) and EtOAc (100 mL) were added. The layers were separated and the aqueous layer was extracted with EtOAc (3×50 mL). The combined organic layers were dried over sodium sulfate, filtered and concentrated under reduced pressure. The crude residue was purified by flash chromatography over silica gel column (CyHex/EtOAc) to afford N-[(1R)-2-[tert-butyl(dimethyl)silyl]oxy-1-(4-methoxyphenyl)ethyl]pyridine-2-carboxamide (20.5 g, 88.9% yield) as a yellow oil. ¹H NMR (400 MHz, DMSO-d6) δ 8.94 (d, J=8.7 Hz, 1H), 8.66 (dt, J 4.8, 1.4 Hz, 1H), 8.07-7.93 (m, 2H), 7.61 (ddd, J=6.8, 4.8, 2.3 Hz, 1H), 7.37-7.27 (m, 2H), 6.95-6.83 (m, 2H), 5.06 (dt, J=8.7, 5.7 Hz, 1H), 3.96-3.82 (m, 2H), 3.72 (s, 3H), 0.81 (s, 9H), -0.04 (d, J=9.2 Hz, 6H).

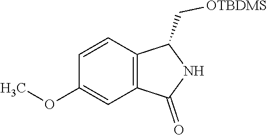 [SMILES: COc1ccc2c(c1)C(=O)N[C@H]2CO[Si](C)(C)C(C)(C)C]

### (3R)-3-[[tert-butyl(dimethyl)silyl]oxymethyl]-6-methoxy-isoindolin-1-one

In an autoclave were charged 2,2-dimethylpropanoic acid (39.57 g, 0.387 mol), isopropyl (N{E})-N-isopropoxycarbonyliminocarbamate (76 mL, 0.387 mol), silver carbonate (107.60 g, 0.387 mol), cobalt(2+) acetate hydrate (1:2:4) (9.65 g, 38.7 mmol), N-[(1S)-1-[4-(trifluoromethyl)phenyl]ethyl]pyridine-2-carboxamide (57.0 g, 0.194 mol) and 2,2,2-trifluoroethanol (330 mL). The reaction mixture was stirred at 110° C. for 24 hours then filtered over Celite and washed with EtOAc. The filtrate was concentrated under reduced pressure and the crude residue was purified by flash chromatography over silica gel column (CyHex/EtOAc) to afford (5 g, 24.4% yield) as yellow oil. ¹H NMR (400 MHz, DMSO-d6) δ 8.54 (s, 1H), 7.50 (d, J=7.9 Hz, 1H), 7.16-7.09 (m, 2H), 4.50 (t, J=5.5 Hz, 1H), 3.81 (s, 3H), 3.79-3.75 (m, 1H), 3.68 (dd, J=10.1, 5.7 Hz, 1H), 0.80 (s, 9H), −0.00 (d, J=11.8 Hz, 6H).

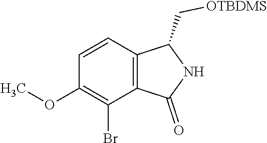 [SMILES: COc1ccc2c(c1Br)C(=O)N[C@H]2CO[Si](C)(C)C(C)(C)C]

### (3R)-7-bromo-3-[[tert-butyl(dimethyl)silyl]oxymethyl]-6-methoxy-isoindolin-1-one

To a solution of (3R)-3-[[tert-butyl(dimethyl)silyl]oxymethyl]-6-methoxy-isoindolin-1-one (8.0 g, 26.02 mmol) in acetonitrile (71 mL) at ambient temperature under nitrogen was added 1-bromopyrrolidine-2,5-dione (5.25 g, 28.62 mmol). The reaction mixture was stirred at ambient temperature for 16 hours then concentrated under reduced pressure. The crude residue was purified by flash chromatography over silica gel column (CyHex/EtOAc) to afford (3R)-7-bromo-3-[[tert-butyl(dimethyl)silyl]oxymethyl]-6-methoxy-isoindolin-1-one (3.4 g, 33.8% yield) as a yellow oil. ¹H NMR (400 MHz, DMSO-d6) δ 8.67 (s, 1H), 7.54 (d, J=8.3 Hz, 1H), 7.32 (d, J=8.3 Hz, 1H), 4.46 (t, J=5.2 Hz, 1H), 3.88 (s, 3H), 3.79 (dd, J=10.1, 4.9 Hz, 1H), 3.71-3.63 (m, 1H), 0.79 (s, 8H), −0.01 (d, J=15.1 Hz, 6H).

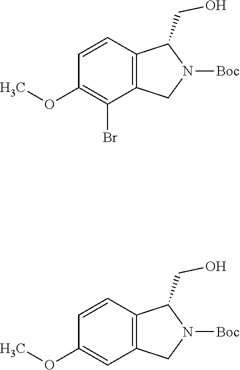 [SMILES: COc1ccc2c(c1Br)CN(C(=O)OC(C)(C)C)[C@H]2CO ; COc1ccc2c(c1)CN(C(=O)OC(C)(C)C)[C@H]2CO]

### tert-butyl (1R)-4-bromo-1-(hydroxymethyl)-5-methoxy-isoindoline-2-carboxylate

### tert-butyl (1R)-1-(hydroxymethyl)-5-methoxy-2,3-dihydro-1H-isoindole-2-carboxylate

To a solution of (3R)-7-bromo-3-[[tert-butyl(dimethyl)silyl]oxymethyl]-6-methoxy-isoindolin-1-one (4.0 g, 10.35 mmol) in THF (50 mL) at 0° C. under nitrogen were successively added chloro(trimethyl)silane (3.94 mL, 31.06 mmol) and lithium aluminium hydride 2M in THF (25.88 mL, 51.77 mmol). The reaction mixture was stirred at reflux for 16 hours then Fieser quench was performed. The crude residue was taken up in THF (50 mL). Di-tert-butyl dicarbonate (2.711 g, 12.42 mmol) and N-ethyl-N-isopropyl-propan-2-amine (5.42 mL, 31.06 mmol) were added. The reaction mixture was stirred at ambient temperature for 1 hour then concentrated under reduced pressure. The crude residue was purified by flash chromatography over silica gel column (DCM/MeOH) to afford a mixture of tert-butyl (1R)-4-bromo-1-(hydroxymethyl)-5-methoxy-isoindoline-2-carboxylate and tert-butyl (1R)-1-(hydroxymethyl)-5-methoxy-2,3-dihydro-1H-isoindole-2-carboxylate (1 g) as a purple foam. ¹H NMR (400 MHz, DMSO) δ 7.31 (t, J=7.8 Hz, 1H), 7.25 (t, J=8.1 Hz, 1H), 7.03 (d, J=8.3 Hz, 1H), 6.88 (s, 1H), 6.83 (dd, J=8.2, 2.4 Hz, 1H), 4.92 (d, J=14.3 Hz, 1H), 4.85-4.64 (m, 2H), 4.63-4.29 (m, 4H), 3.85 (s, 3H), 3.74 (s, 5H), 3.62 (dt, J=10.7, 5.6 Hz, 1H), 1.46 (s, 4H), 1.45 (s, 11H).

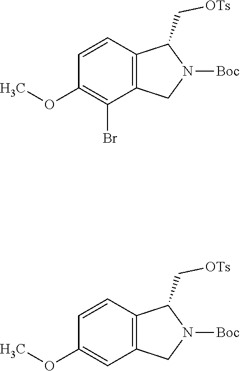 [SMILES: COc1ccc2c(c1Br)CN(C(=O)OC(C)(C)C)[C@H]2COS(=O)(=O)c1ccc(C)cc1 ; COc1ccc2c(c1)CN(C(=O)OC(C)(C)C)[C@H]2COS(=O)(=O)c1ccc(C)cc1]

### tert-butyl (1R)-4-bromo-5-methoxy-1-(p-tolylsulfonyloxymethyl)isoindoline-2-carboxylate

### tert-butyl (1R)-5-methoxy-1-{[(4-methylbenzenesulfonyl)oxy]methyl}-2,3-dihydro-1H-isoindole-2-carboxylate

To a solution of tert-butyl (1R)-4-bromo-1-(hydroxymethyl)-5-methoxy-isoindoline-2-carboxylate (1.0 g, 2.79 mmol) in DCM (10 mL) at 0° C. under nitrogen were successively added DMAP (34 mg, 0.28 mmol), triethylamine (1.17 mL, 8.37 mmol) and 4-methylbenzenesulfonyl chloride (798 mg, 4.19 mmol). The reaction mixture was stirred from 0° C. to ambient temperature for 2 hours then saturated NH₄Cl solution (25 mL) and EtOAc (25 mL) were added. The layers were separated and the aqueous layer was extracted with EtOAc (2×25 mL). The combined organic layers were dried over sodium sulfate, filtered and concentrated under reduced pressure. The crude residue was purified by flash chromatography over silica gel column (CyHex/EtOAc) to afford a mixture of tert-butyl (1R)-4-bromo-5-methoxy-1-(p-tolylsulfonyloxymethyl)isoindoline-2-carboxylate and tert-butyl (1R)-5-methoxy-1-{[(4-methylbenzenesulfonyl)oxy]methyl}-2,3-dihydro-1H-isoindole-2-carboxylate (1 g) as a pink oil. ¹H NMR (400 MHz, DMSO) δ 7.68-7.54 (m, 2.0H), 7.42 (t, J=6.7 Hz, 2.0H), 7.25 (d, J=8.3 Hz, 0.6H), 7.16 (dd, J=8.5, 5.1 Hz, 0.4H), 7.02 (d, J=8.4 Hz, 0.6H), 6.89 (d, J=5.2 Hz, 0.4H), 6.82 (d, J=8.5 Hz, 0.4H), 5.16 (s, 0.6H), 5.02 (s, 0.4H), 4.58-4.44 (m, 1.4H), 4.43-4.30 (m, 1.2H), 4.24 (dd, J=15.9, 6.6 Hz, 0.6H), 3.86 (s, 1.8H), 3.75 (s, 1.0H), 2.41 (s, 2.8H), 1.37 (d, J=23.1 Hz, 11.0H).

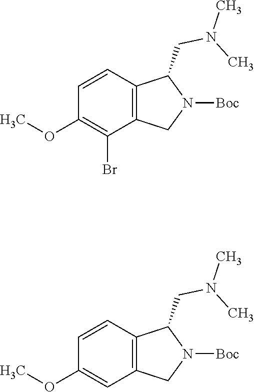

### tert-butyl (1R)-4-bromo-1-[(dimethylamino)methyl]-5-methoxy-isoindoline-2-carboxylate

### tert-butyl (1R)-1-[(dimethylamino)methyl]-5-methoxy-isoindoline-2-carboxylate

A solution of tert-butyl (1R)-4-bromo-5-methoxy-1-(p-tolylsulfonyloxymethyl)isoindoline-2-carboxylate (200 mg, 0.39 mmol) in N-methylmethanamine (2M in THF, 8 mL, 15.61 mmol) was stirred at 70° C. for 16 hours. Saturated NaHCO₃ solution (15 mL) and EtOAc (15 mL) were added. The layers were separated and the aqueous layer was extracted with EtOAc (2×15 mL). The combined organic layers were dried over sodium sulfate, filtered and concentrated under reduced pressure to afford a mixture of tert-butyl (1R)-4-bromo-1-[(dimethylamino)methyl]-5-methoxy-isoindoline-2-carboxylate and tert-butyl (1R)-1-[(dimethylamino)methyl]-5-methoxy-isoindoline-2-carboxylate (150 mg) as an orange oil. ¹H NMR (400 MHz, DMSO) δ ppm 7.38-7.33 (m, 1H), 7.30 (d, J=8.4 Hz, 1H), 7.03 (d, J=8.4 Hz, 1H), 6.88 (s, 1H), 6.82 (dd, J=8.4, 2.5 Hz, 1H), 5.02-4.90 (m, 1H), 4.81 (s, 1H), 4.65-4.49 (m, 2H), 4.41 (q, J=17.1 Hz, 2H), 3.85 (s, 3H), 3.74 (s, 2H), 2.69-2.55 (m, 2H), 2.46-2.29 (m, 1H), 2.21-2.09 (m, 10H), 1.46 (s, 11H).

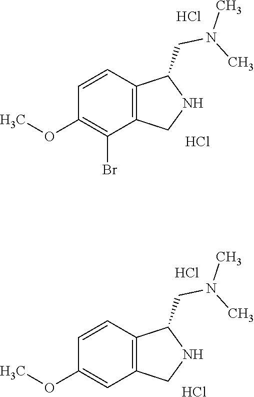

### (1-[(1R)-4-bromo-5-methoxy-isoindolin-1-yl]-N,N-dimethyl-methanamine dihydrochloride

### 1-[(1R)-5-methoxyisoindolin-1-yl]-N,N-dimethyl-methanamine dihydrochloride

A mixture of tert-butyl (1R)-4-bromo-1-[(dimethylamino)methyl]-5-methoxy-isoindoline-2-carboxylate (150 mg, 0.39 mmol) and 4M hydrogen chloride in dioxane (2 mL, 7.79 mmol) was stirred at ambient temperature for 1 hour then concentrated under reduced pressure to afford a mixture of (1-[(1R)-4-bromo-5-methoxy-isoindolin-1-yl]-N,N-dimethyl-methanamine dihydrochloride and 1-[(1R)-5-methoxyisoindolin-1-yl]-N,N-dimethyl-methanamine dihydrochloride (140 mg) as a grey gum which was engaged in the next step without further purification. LCMS (ESI) m/z 285/287 [M+H]⁺ for (1-[(1R)-4-bromo-5-methoxy-isoindolin-1-yl]-N,N-dimethyl-methanamine dihydrochloride and m/z 207 [M+H]⁺ for 1-[(1R)-5-methoxyisoindolin-1-yl]-N,N-dimethyl-methanamine dihydrochloride.

### Example 16: Synthesis of Compounds

Compounds of Formula (I) (e.g., Formula (Ia), Formula (Ib), Formula (Ic), Formula (Id)) were synthesized according to the procedures described below.

### Synthesis of Compound 38

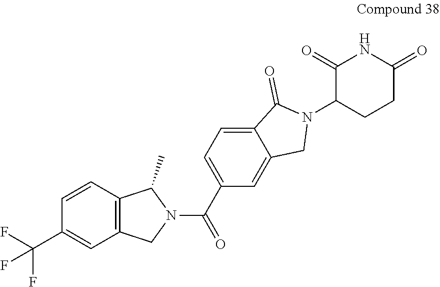 [SMILES: C[C@H]1c2ccc(C(F)(F)F)cc2CN1C(=O)c1ccc2c(c1)CN(C1CCC(=O)NC1=O)C2=O]

### 3-[5-[(1S)-1-methyl-5-(trifluoromethyl)isoindoline-2-carbonyl]-1-oxo-isoindolin-2-yl]piperidine-2,6-dione

Corresponding isoindoline was synthesized following Isoindoline Synthesis A then coupled to CRBN core 1 following Coupling Procedure A to afford 3-[5-[(1S)-1-methyl-5-(trifluoromethyl)isoindoline-2-carbonyl]-1-oxo-isoindolin-2-yl]piperidine-2,6-dione (61 mg, 62% yield) as a white solid. ¹H NMR (500 MHz, DMSO-d₆) δ ppm 11.34-10.71 (m, 1H), 7.97-7.77 (m, 2H), 7.76-7.45 (m, 4H), 5.54 (br d, J=6.4 Hz, 1H), 5.21-5.07 (m, 1H), 5.06-4.32 (m, 4H), 2.99-2.56 (m, 2H), 2.48-1.99 (m, 2H), 1.83-0.79 (m, 3H). LCMS (ESI) m/z 472 [M+H]⁺.

### Synthesis of Compound 31

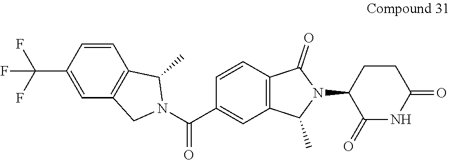 [SMILES: C[C@@H]1c2cc(C(=O)N3Cc4cc(C(F)(F)F)ccc4[C@@H]3C)ccc2C(=O)N1[C@H]1CCC(=O)NC1=O]

### (3S)-3-[(3R)-3-methyl-5-[(1S)-1-methyl-5-(trifluoromethyl)isoindoline-2-carbonyl]-1-oxo-isoindolin-2-yl]piperidine-2,6-dione

Corresponding isoindoline was synthesized following Isoindoline Synthesis A then coupled to CRBN core 2 following Coupling Procedure B to afford (3 S)-3-[(3R)-3-methyl-5-[(1S)-1-methyl-5-(trifluoromethyl)isoindoline-2-carbonyl]-1-oxo-isoindolin-2-yl]piperidine-2,6-dione (90.1 mg, 56% yield) as a white solid. ¹H NMR (500 MHz, DMSO-d₆) δ ppm 10.97 (s, 1H), 7.89 (s, 1H), 7.86-7.74 (m, 1H), 7.72-7.47 (m, 4H), 5.59-5.27 (m, 1H), 5.09-4.95 (m, 1H), 4.93-4.71 (m, 2H), 4.59 (d, J=15.16 Hz, 1H), 2.91-2.77 (m, 1H), 2.75-2.57 (m, 2H), 2.06-1.99 (m, 1H), 1.59 (d, J=6.36 Hz, 2H), 1.52-1.40 (m, 3H), 1.08 (br d, J=6.36 Hz, 1H). LCMS (ESI) m/z 486 [M+H]⁺.

### Synthesis of Compound 21

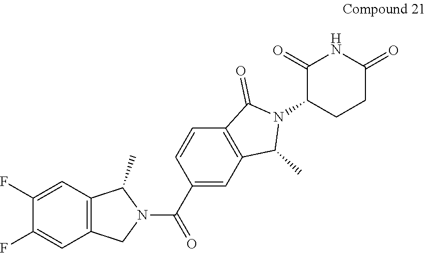 [SMILES: C[C@@H]1c2cc(C(=O)N3Cc4cc(F)c(F)cc4[C@@H]3C)ccc2C(=O)N1[C@H]1CCC(=O)NC1=O]

### (3S)-3-[(3R)-5-[(1S)-5,6-difluoro-1-methyl-2,3-dihydro-1H-isoindole-2-carbonyl]-3-methyl-1-oxo-2,3-dihydro-1H-isoindol-2-yl]piperidine-2,6-dione

Corresponding isoindoline was synthesized following Isoindoline Synthesis A then coupled to CRBN core 2 following Coupling Procedure D. The product was cyclized following Cyclization Procedure to afford (3S)-3-[(3S)-5-[(1S)-5,6-difluoro-1-methyl-2,3-dihydro-1H-isoindole-2-carbonyl]-3-methyl-1-oxo-2,3-dihydro-1H-isoindol-2-yl]piperidine-2,6-dione (39 mg, 66% yield) as a white solid. ¹H NMR (600 MHz, DMSO-d₆) δ ppm 10.97 (s, 1H), 7.91-7.85 (m, 1H), 7.79-7.73 (m, 1H), 7.70-7.64 (m, 1H), 7.54 (br dd, J=9.98, 7.63 Hz, 1H), 7.33 (br dd, J=9.98, 7.78 Hz, 1H), 5.43 (q, J=5.67 Hz, 1H), 4.95-4.87 (m, 1H), 4.83-4.71 (m, 3H), 4.45 (br d, J=14.53 Hz, 1H), 2.88-2.79 (m, 1H), 2.71-2.57 (m, 2H), 2.05-1.97 (m, 1H), 1.55 (d, J=6.46 Hz, 2H), 1.45 (br d, J=6.75 Hz, 3H). LCMS (ESI) m/z 454 [M+H]⁺.

### Synthesis of Compound 26

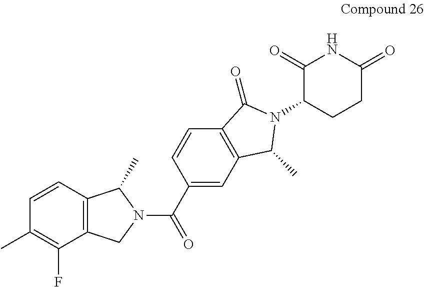 [SMILES: Cc1ccc2c(c1F)CN(C(=O)c1ccc3c(c1)[C@@H](C)N([C@H]1CCC(=O)NC1=O)C3=O)[C@H]2C]

### (3S)-3-[(3R)-5-[(1S)-4-fluoro-1,5-dimethyl-isoindoline-2-carbonyl]-3-methyl-1-oxo-isoindolin-2-yl]piperidine-2,6-dione

Corresponding isoindoline was synthesized following Isoindoline Synthesis D and coupled to CRBN core following Coupling Procedure D. The coupling product was cyclized following Cyclization Procedure to afford ((3S)-3-[(3R)-5-[(1S)-4-fluoro-1,5-dimethyl-isoindoline-2-carbonyl]-3-methyl-1-oxo-isoindolin-2-yl]piperidine-2,6-dione (17 mg, 44% yield) as a white solid. ¹H NMR (600 MHz, DMSO-d₆) δ ppm 10.96 (s, 1H), 7.92-7.86 (m, 1H), 7.76 (d, J=7.48 Hz, 1H), 7.70 (d, J=7.92 Hz, 1H), 7.28-7.22 (m, 1H), 7.16-6.95 (m, 1H), 5.50-5.21 (m, 1H), 5.06-4.91 (m, 1H), 4.87-4.72 (m, 2H), 4.53 (d, J=14.38 Hz, 1H), 2.90-2.77 (m, 1H), 2.73-2.56 (m, 2H), 2.28-2.24 (m, 1H), 2.21 (s, 2H), 2.06-1.98 (m, 1H), 1.54 (d, J=6.46 Hz, 2H), 1.48-1.41 (m, 3H), 1.05-1.02 (m, 1H). LCMS (ESI) m/z 450 [M+H]⁺.

### Synthesis of Compound 27

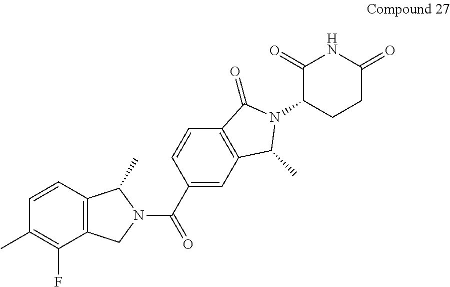 [SMILES: Cc1ccc2c(c1F)CN(C(=O)c1ccc3c(c1)[C@@H](C)N([C@H]1CCC(=O)NC1=O)C3=O)[C@H]2C]

### (3S)-3-[(3R)-5-[(1S)-6-fluoro-1-methyl-isoindoline-2-carbonyl]-3-methyl-1-oxo-isoindolin-2-yl]piperidine-2,6-dione

Corresponding isoindoline was synthesized following Isoindoline Synthesis A and coupled to CRBN core 2 following Coupling Procedure D. The coupling product was cyclized following Cyclization Procedure to afford (3S)-3-[(3R)-5-[(1S)-6-fluoro-1-methyl-isoindoline-2-carbonyl]-3-methyl-1-oxo-isoindolin-2-yl]piperidine-2,6-dione (28 mg, 58% yield) as a white solid. ¹H NMR (600 MHz, DMSO-d₆) δ ppm 10.96 (s, 1H), 7.92-7.83 (m, 1H), 7.80-7.72 (m, 1H), 7.71-7.64 (m, 1H), 7.49-7.23 (m, 2H), 7.19-7.07 (m, 1H), 5.54-5.17 (m, 1H), 5.00-4.68 (m, 3H), 4.45 (d, J=14.23 Hz, 1H), 2.92-2.77 (m, 1H), 2.74-2.55 (m, 2H), 2.06-1.96 (m, 1H), 1.56 (d, J=6.46 Hz, 2H), 1.45 (br d, J=6.60 Hz, 3H), 1.05 (br d, J=6.31 Hz, 1H). LCMS (ESI) m/z 436 [M+H]⁺.

### Synthesis of Compound 36

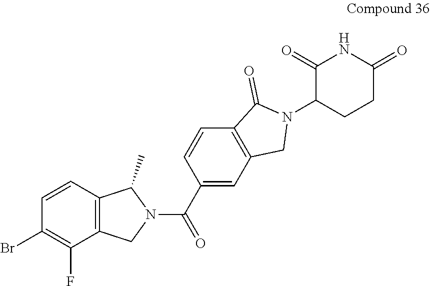 [SMILES: C[C@H]1c2ccc(Br)c(F)c2CN1C(=O)c1ccc2c(c1)CN(C1CCC(=O)NC1=O)C2=O]

### 3-[5-[(1S)-5-bromo-4-fluoro-1-methyl-isoindoline-2-carbonyl]-1-oxo-isoindolin-2-yl]piperidine-2,6-dione

Corresponding isoindoline was synthesized following Isoindoline Synthesis B and coupled to CRBN core 1 following Coupling Procedure A to afford 3-[5-[(1S)-5-bromo-4-fluoro-1-methyl-isoindoline-2-carbonyl]-1-oxo-isoindolin-2-yl]piperidine-2,6-dione (43 mg, 50% yield) as a white solid. ¹H NMR (500 MHz, DMSO-d₆) δ ppm 11.01 (s, 1H), 7.83 (d, J=7.58 Hz, 2H), 7.71 (br d, J=7.58 Hz, 1H), 7.69-7.62 (m, 1H), 7.23 (d, J=8.07 Hz, 1H), 5.57-5.21 (m, 1H), 5.15 (br dd, J=13.20, 3.18 Hz, 1H), 5.10-4.87 (m, 1H), 4.72-4.30 (m, 3H), 3.03-2.84 (m, 1H), 2.70-2.56 (m, 1H), 2.43 (qd, J=13.20, 4.40 Hz, 1H), 2.13-1.97 (m, 1H), 1.56 (d, J=6.36 Hz, 2H), 1.06 (br d, J=6.11 Hz, 1H). LCMS (ESI) m/z 502 [M+H]⁺

### Synthesis of Compound 19

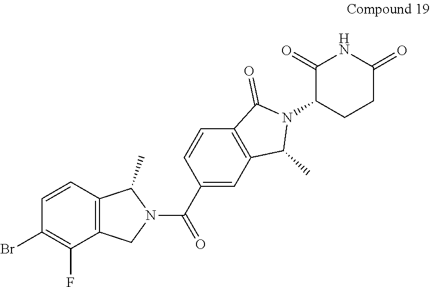 [SMILES: C[C@@H]1c2cc(C(=O)N3Cc4c(ccc(Br)c4F)[C@@H]3C)ccc2C(=O)N1[C@H]1CCC(=O)NC1=O]

### (3S)-3-[(3R)-5-[(1S)-5-bromo-4-fluoro-1-methyl-isoindoline-2-carbonyl]-3-methyl-1-oxo-isoindolin-2-yl]piperidine-2,6-dione

Corresponding isoindoline was synthesized following Isoindoline Synthesis B and coupled to CRBN core 2 following Coupling Procedure B to afford (3 S)-3-[(3R)-5-[(1S)-5-bromo-4-fluoro-1-methyl-isoindoline-2-carbonyl]-3-methyl-1-oxo-isoindolin-2-yl]piperidine-2,6-dione (40 mg, 77% yield) as a white solid. ¹H NMR (600 MHz, DMSO-d₆) δ ppm 10.96 (s, 1H), 7.89 (s, 1H), 7.77 (d, J=7.48 Hz, 1H), 7.67 (s, 2H), 7.23 (d, J=8.07 Hz, 1H), 5.56-5.23 (m, 1H), 5.17-4.97 (m, 1H), 4.95-4.55 (m, 3H), 2.92-2.57 (m, 3H), 2.05-1.93 (m, 1H), 1.56 (d, J=6.46 Hz, 2H), 1.50-1.38 (m, 3H), 1.05 (br d, J=6.46 Hz, 1H). LCMS (ESI) m/z 514 [M+H]⁺.

### Synthesis of Compound 30

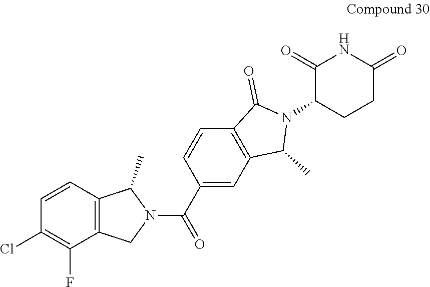 [SMILES: C[C@@H]1c2cc(C(=O)N3Cc4c(ccc(Cl)c4F)[C@@H]3C)ccc2C(=O)N1[C@H]1CCC(=O)NC1=O]

### (3S)-3-[(3R)-5-[(1S)-5-chloro-4-fluoro-1-methyl-isoindoline-2-carbonyl]-3-methyl-1-oxo-isoindolin-2-yl]piperidine-2,6-dione

Corresponding isoindoline was synthesized following Isoindoline Synthesis C and coupled to CRBN core 2 following Coupling Procedure B to afford (3 S)-3-[(3R)-5-[(1S)-5-chloro-4-fluoro-1-methyl-isoindoline-2-carbonyl]-3-methyl-1-oxo-isoindolin-2-yl]piperidine-2,6-dione (25 mg, 53.4% yield) as a white solid. ¹H NMR (500 MHz, DMSO-d₆) δ ppm 10.98 (s, 1H), 7.71-7.62 (m, 3H), 7.51-7.50 (m, 2H), 5.50 (q, J=5.79 Hz, 1H), 4.92-4.70 (m, 3H), 4.51 (d, J=15.16 Hz, 1H), 2.90-2.80 (m, 1H), 2.72-2.58 (m, 2H), 2.05-2.00 (m, 1H), 1.61 (d, J=6.60 Hz, 3H), 1.53-1.42 (m, 3H). LCMS (ESI) m/z 470 [M+H]⁺.

### Synthesis of Compound 13

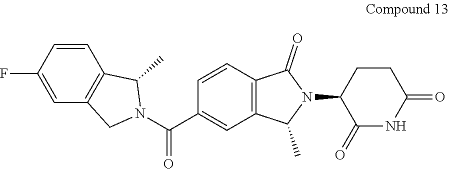 [SMILES: C[C@@H]1c2cc(C(=O)N3Cc4cc(F)ccc4[C@@H]3C)ccc2C(=O)N1[C@H]1CCC(=O)NC1=O]

### (3S)-3-[(3R)-5-[(1S)-5-fluoro-1-methyl-isoindoline-2-carbonyl]-3-methyl-1-oxo-isoindolin-2-yl]piperidine-2,6-dione

Corresponding isoindoline was synthesized following Isoindoline Synthesis A and coupled to CRBN core 2 following Coupling Procedure D. The coupling product was cyclized following Cyclization Procedure to afford (3S)-3-[(3R)-5-[(1S)-5-fluoro-1-methyl-isoindoline-2-carbonyl]-3-methyl-1-oxo-isoindolin-2-yl]piperidine-2,6-dione (19 mg, 61.8% yield) as a white solid. ¹H NMR (600 MHz, DMSO-d₆) δ ppm 10.96 (br s, 1H), 7.88-7.87 (m, 1H), 7.76 (d, J=7.8 Hz, 1H), 7.69-7.67 (m, 1H), 7.42-7.08 (m, 3H), 5.42 (q, J=5.8 Hz, 1H), 4.96-4.47 (m, 4H), 2.84-2.81 (m, 1H), 2.68-2.60 (m, 2H), 2.03-2.00 (m, 1H), 1.55-1.43 (m, 5H), 1.03 (d, J=6.3 Hz, 1H). LCMS (ESI) m/z 436 [M+H]⁺.

### Synthesis of Compound 9

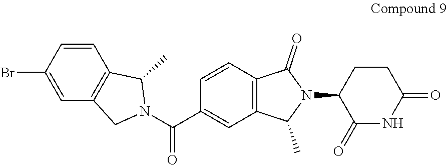 [SMILES: C[C@@H]1c2cc(C(=O)N3Cc4cc(Br)ccc4[C@@H]3C)ccc2C(=O)N1[C@H]1CCC(=O)NC1=O]

### (3S)-3-[(3R)-5-[(1S)-5-bromo-1-methyl-isoindoline-2-carbonyl]-3-methyl-1-oxo-isoindolin-2-yl]piperidine-2,6-dione

Corresponding isoindoline was synthesized following Isoindoline Synthesis A and coupled to CRBN core 2 following Coupling Procedure B to afford (3 S)-3-[(3R)-5-[(1S)-5-bromo-1-methyl-isoindoline-2-carbonyl]-3-methyl-1-oxo-isoindolin-2-yl]piperidine-2,6-dione (38 mg, 46% yield) as a white solid. ¹H NMR (500 MHz, DMSO-d₆) δ ppm 10.97 (s, 1H), 7.88 (s, 1H), 7.76 (d, J=7.58 Hz, 1H), 7.71-7.64 (m, 1H), 7.53-7.48 (m, 1H), 7.47 (s, 1H), 7.37 (d, J=8.07 Hz, 1H), 5.57-5.13 (m, 1H), 4.95 (br d, J=14.92 Hz, 1H), 4.86-4.43 (m, 3H), 2.90-2.79 (m, 1H), 2.74-2.58 (m, 2H), 2.06-1.98 (m, 1H), 1.54 (d, J=6.36 Hz, 2H), 1.46 (br d, J=6.85 Hz, 3H), 1.03 (d, J=6.36 Hz, 1H). LCMS (ESI) m/z 496 [M+H]⁺.

### Synthesis of Compound 22

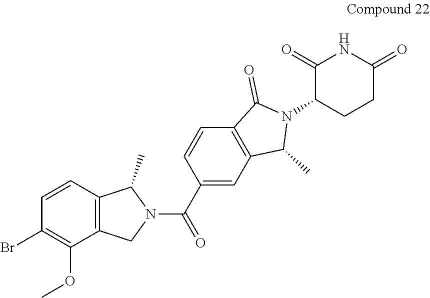 [SMILES: COc1c(Br)ccc2c1CN(C(=O)c1ccc3c(c1)[C@@H](C)N([C@H]1CCC(=O)NC1=O)C3=O)[C@H]2C]

### (3S)-3-[(3R)-5-[(1S)-5-bromo-4-methoxy-1-methyl-isoindoline-2-carbonyl]-3-methyl-1-oxo-isoindolin-2-yl]piperidine-2,6-dione

Corresponding isoindoline was synthesized following Isoindoline Synthesis E then coupled to CRBN core 2 following Coupling Procedure B to afford (3 S)-3-[(3R)-5-[(1S)-5-bromo-4-methoxy-1-methyl-isoindoline-2-carbonyl]-3-methyl-1-oxo-isoindolin-2-yl]piperidine-2,6-dione (52 mg, 62% yield) as a white solid. ¹H NMR (500 MHz, DMSO-d₆) δ ppm 10.97 (s, 1H), 7.90-7.78 (m, 1H), 7.77-7.76 (m, 1H), 7.73-7.71 (m, 1H), 7.59-7.57 (m, 1H), 7.11-6.97 (m, 1H), 5.44-5.21 (m, 1H), 5.13-4.62 (m, 4H), 3.91-3.72 (m, 3H), 2.90-2.82 (m, 1H), 2.71-2.59 (m, 2H), 2.08-1.85 (m, 1H), 1.55-1.34 (m, 5H), 1.04 (d, J=6.4 Hz, 1H). LCMS (ESI) m/z 526 [M+H]⁺.

### Synthesis of Compound 37

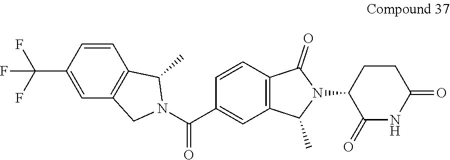 [SMILES: C[C@@H]1c2cc(C(=O)N3Cc4cc(C(F)(F)F)ccc4[C@@H]3C)ccc2C(=O)N1[C@@H]1CCC(=O)NC1=O]

Corresponding isoindoline was synthesized following Isoindoline Synthesis A and coupled to the corresponding carboxylic acid following Coupling Procedure D. The coupling product was cyclized following Cyclization Procedure to afford (3R)-3-[(3R)-3-methyl-5-[(1S)-1-methyl-5-(trifluoromethyl)isoindoline-2-carbonyl]-1-oxo-isoindolin-2-yl]piperidine-2,6-dione (91 mg, 58.3% yield) as a white powder. ¹H NMR (500 MHz, DMSO-d₆) δ ppm 10.94 (s, 1H), 7.90 (s, 1H), 7.85-7.74 (m, 1H), 7.69 (br d, J=7.82 Hz, 2H), 7.67-7.48 (m, 2H), 5.68-5.26 (m, 1H), 5.02 (br d, J=15.65 Hz, 1H), 4.93-4.71 (m, 2H), 4.59 (d, J=14.92 Hz, 1H), 2.88-2.55 (m, 3H), 2.07-1.99 (m, 1H), 1.59 (d, J=6.36 Hz, 2H), 1.53-1.42 (m, 3H), 1.08 (br d, J=6.36 Hz, 1H). LCMS (ESI) m/z 486 [M+H]⁺.

### Synthesis of Compound 25

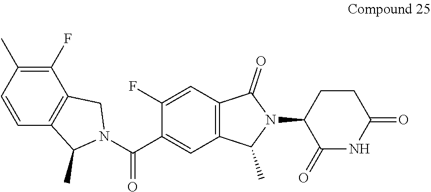 [SMILES: Cc1ccc2c(c1F)CN(C(=O)c1cc3c(cc1F)C(=O)N([C@H]1CCC(=O)NC1=O)[C@@H]3C)[C@H]2C]

Corresponding isoindoline was synthesized following Isoindoline Synthesis D and coupled to CRBN core 3 following Coupling Procedure B to afford (3 S)-3-[(1R)-5-fluoro-6-[(1S)-4-fluoro-1,5-dimethyl-isoindoline-2-carbonyl]-1-methyl-3-oxo-isoindolin-2-yl]piperidine-2,6-dione (14 mg, 84.7% yield) as a white solid. ¹H NMR (500 MHz, DMSO-d₆) δ ppm 10.98 (d, J=2.93 Hz, 1H), 7.94-7.83 (m, 1H), 7.64 (dd, J=17.24, 8.19 Hz, 1H), 7.32-7.21 (m, 1H), 7.19-6.95 (m, 1H), 5.50-5.03 (m, 1H), 5.03-4.48 (m, 4H), 2.91-2.75 (m, 1H), 2.70-2.57 (m, 2H), 2.21 (s, 3H), 2.08-1.95 (m, 1H), 1.55 (d, J=6.36 Hz, 2H), 1.45 (d, J=6.60 Hz, 3H), 1.11 (d, J=6.36 Hz, 1H). LCMS (ESI) m/z 468 [M+H]⁺.

### Synthesis of Compound 20

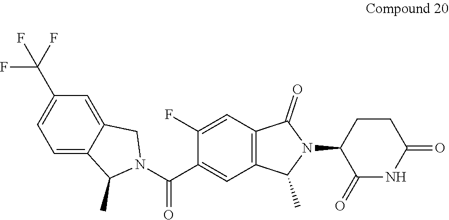 [SMILES: C[C@@H]1c2cc(C(=O)N3Cc4cc(C(F)(F)F)ccc4[C@@H]3C)c(F)cc2C(=O)N1[C@H]1CCC(=O)NC1=O]

### (3S)-3-[(1R)-5-fluoro-1-methyl-6-[(1S)-1-methyl-5-(trifluoromethyl)isoindoline-2-carbonyl]-3-oxo-isoindolin-2-yl]piperidine-2,6-dione

Corresponding isoindoline was synthesized following Isoindoline Synthesis A and coupled to CRBN core 3 following Coupling Procedure B to afford (3S)-3-[(1R)-5-fluoro-1-methyl-6-[(1S)-1-methyl-5-(trifluoromethyl)isoindoline-2-carbonyl]-3-oxo-isoindolin-2-yl]piperidine-2,6-dione (66.2 mg, 69.0% yield) as a white solid. ¹H NMR (600 MHz, DMSO-d₆,) δ ppm 10.98 (s, 1H), 7.91-7.32 (m, 5H), 5.55-4.44 (m, 5H), 2.92-2.78 (m, 1H), 2.71-2.58 (m, 2H), 2.15-1.98 (m, 1H), 1.59 (d, J=6.5 Hz, 2H), 1.49-1.39 (m, 3H), 1.2 (d, J=6.5 Hz, 1H). LCMS (ESI) m/z 504 [M+H]⁺.

### Synthesis of Compound 35

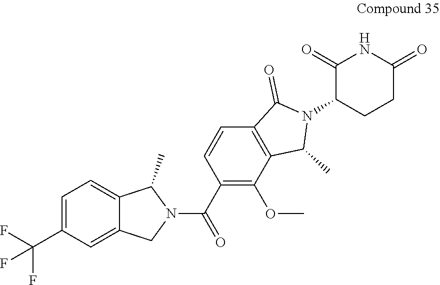 [SMILES: COc1c(C(=O)N2Cc3cc(C(F)(F)F)ccc3[C@@H]2C)ccc2c1[C@@H](C)N([C@H]1CCC(=O)NC1=O)C2=O]

### (3S)-3-[(3R)-4-methoxy-3-methyl-5-[(1S)-1-methyl-5-(trifluoromethyl)isoindoline-2-carbonyl]-1-oxo-isoindolin-2-yl]piperidine-2,6-dione

Corresponding isoindoline was synthesized following Isoindoline Synthesis A and coupled to CRBN core 4 following Coupling Procedure B to afford as a white powder (3S)-3-[(3R)-4-methoxy-3-methyl-5-[(1S)-1-methyl-5-(trifluoromethyl)isoindoline-2-carbonyl]-1-oxo-isoindolin-2-yl]piperidine-2,6-dione (50 mg, 43.0% yield) as a white solid. ¹H NMR (500 MHz, DMSO-d₆) δ ppm 10.98 (s, 1H), 7.71-7.62 (m, 3H), 7.51-7.50 (m, 2H), 5.50 (q, J=5.79 Hz, 1H), 4.92-4.70 (m, 3H), 4.51 (d, J=15.16 Hz, 1H), 3.95-3.81 (m, 3H), 2.90-2.80 (m, 1H), 2.72-2.58 (m, 2H), 2.05-2.00 (m, 1H), 1.61 (d, J=6.60 Hz, 3H), 1.53-1.42 (m, 3H). LCMS (ESI) m/z 516 [M+H]⁺.

### Synthesis of Compound 33

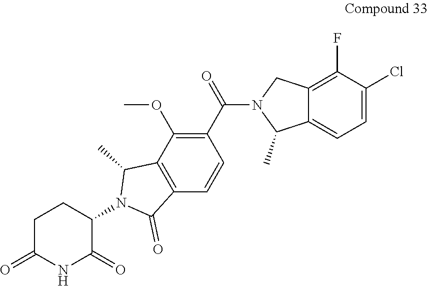 [SMILES: COc1c(C(=O)N2Cc3c(ccc(Cl)c3F)[C@@H]2C)ccc2c1[C@@H](C)N([C@H]1CCC(=O)NC1=O)C2=O]

### (S)-3-((R)-5-((S)-5-chloro-4-fluoro-1-methylisoindoline-2-carbonyl)-4-methoxy-3-methyl-1-oxoisoindolin-2-yl)piperidine-2,6-dione

Corresponding isoindoline was synthesized following Isoindoline Synthesis C and coupled to CRBN core 4 following Coupling Procedure D. The coupling product was cyclized following Cyclization Procedure to afford (3S)-3-[(3R)-5-[(1S)-5-chloro-4-fluoro-1-methyl-isoindoline-2-carbonyl]-4-methoxy-3-methyl-1-oxo-isoindolin-2-yl]piperidine-2,6-dione (45 mg, 64.6% yield). ¹H NMR (500 MHz, DMSO-d₆,) δ ppm 10.98 (s, 1H), 7.62-7.45 (m, 3H), 7.31-7.20 (m, 1H), 5.50-5.41 (m, 1H), 5.20-4.51 (m, 4H), 4.10-3.68 (m, 3H), 3.02-2.81 (m, 1H), 2.80-2.60 (m, 2H), 2.08-1.85 (m, 1H), 1.58 (d, J=6.4 Hz, 2H), 1.48-1.45 (m, 3H), 1.09 (d, J=6.5 Hz, 1H). LCMS (ESI) m/z 500 [M+H]⁺.

### Synthesis of Compound 18

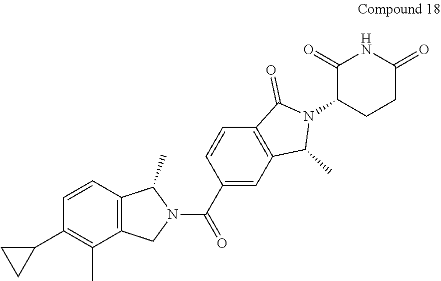 [SMILES: Cc1c(C2CC2)ccc2c1CN(C(=O)c1ccc3c(c1)[C@@H](C)N([C@H]1CCC(=O)NC1=O)C3=O)[C@H]2C]

### (S)-3-((R)-5-((S)-5-cyclopropyl-1,4-dimethylisoindoline-2-carbonyl)-3-methyl-1-oxoisoindolin-2-yl)piperidine-2,6-dione

Corresponding isoindoline was synthesized following isoindoline synthesis F and coupled to CRBN core 2 following Coupling Procedure B to afford (3S)-3-[(3R)-5-[(1S)-5-cyclopropyl-1,4-dimethyl-isoindoline-2-carbonyl]-3-methyl-1-oxo-isoindolin-2-yl]piperidine-2,6-dione (14.1 mg, 17.0% yield) as a white powder. ¹H NMR (600 MHz, DMSO-d₆) δ ppm 11.06-10.88 (m, 1H), 7.92-7.82 (m, 1H), 7.76 (d, J=7.78 Hz, 1H), 7.70 (dd, J=7.78, 0.73 Hz, 1H), 7.09 (d, J=7.78 Hz, 1H), 6.99-6.89 (m, 1H), 5.42 (q, J=6.02 Hz, 1H), 4.97-4.85 (m, 1H), 4.84-4.71 (m, 2H), 4.46 (d, J=14.38 Hz, 1H), 2.91-2.79 (m, 1H), 2.71-2.57 (m, 2H), 2.15 (s, 2H), 2.05-1.97 (m, 1H), 1.99-1.82 (m, 2H), 1.51 (d, J=6.31 Hz, 2H), 1.48-1.41 (m, 3H), 0.95-0.82 (m, 3H), 0.63-0.47 (m, 2H). LCMS (ESI) m/z 472 [M+H]⁺.

### Synthesis of Compound 23

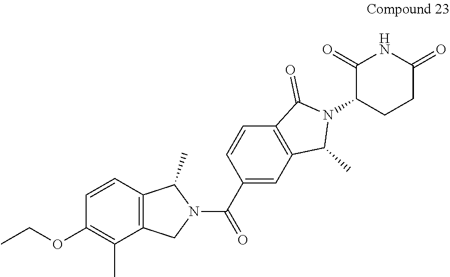 [SMILES: CCOc1ccc2c(c1C)CN(C(=O)c1ccc3c(c1)[C@@H](C)N([C@H]1CCC(=O)NC1=O)C3=O)[C@H]2C]

### (3S)-3-[(3R)-5-[(1S)-5-ethoxy-1,4-dimethyl-isoindoline-2-carbonyl]-3-methyl-1-oxo-isoindolin-2-yl]piperidine-2,6-dione

Corresponding isoindoline was synthesized following isoindoline synthesis G and coupled to CRBN core 2 following Coupling Procedure B to afford (3S)-3-[(3R)-5-[(1S)-5-ethoxy-1,4-dimethyl-isoindoline-2-carbonyl]-3-methyl-1-oxo-isoindolin-2-yl]piperidine-2,6-dione (54 mg, 57% yield) as a white solid. ¹H NMR (500 MHz, DMSO-d₆) δ ppm 10.97 (s, 1H), 7.91-7.83 (m, 1H), 7.77-7.75 (m, 1H), 7.71-7.69 (m, 1H), 7.18-7.02 (m, 1H), 6.91 (d, J=8.6 Hz, 1H), 5.48-5.05 (m, 1H), 4.92-4.42 (m, 4H), 4.09-3.85 (m, 2H), 2.92-2.78 (m, 1H), 2.71-2.60 (m, 2H), 2.13 (s, 1H), 2.11-2.02 (m, 1H), 1.94 (s, 2H), 1.61-1.40 (m, 5H), 1.40-1.31 (m, 3H), 1.00 (d, J=6.4 Hz, 1H). LCMS (ESI) m/z 476 [M+H]⁺.

### Synthesis of Compound 32

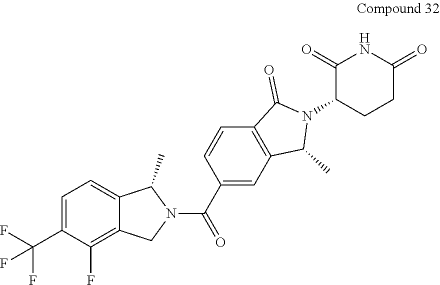 [SMILES: C[C@@H]1c2cc(C(=O)N3Cc4c(ccc(C(F)(F)F)c4F)[C@@H]3C)ccc2C(=O)N1[C@H]1CCC(=O)NC1=O]

### (3S)-3-[(3R)-5-[(1S)-4-fluoro-1-methyl-5-(trifluoromethyl)isoindoline-2-carbonyl]-3-methyl-1-oxo-isoindolin-2-yl]piperidine-2,6-dione

Corresponding isoindoline was synthesized following Isoindoline Synthesis A and coupled to CRBN core 2 following Coupling Procedure B to afford (3 S)-3-[(3R)-5-[(1S)-4-fluoro-1-methyl-5-(trifluoromethyl)isoindoline-2-carbonyl]-3-methyl-1-oxo-isoindolin-2-yl]piperidine-2,6-dione (20 mg, 36.6% yield) as a white solid. ¹H NMR (600 MHz, DMSO-d₆) δ ppm 11.09-10.84 (m, 1H), 7.92-7.86 (m, 1H), 7.81-7.73 (m, 2H), 7.72-7.65 (m, 1H), 7.48 (d, J=7.92 Hz, 1H), 5.59 (br d, J=6.31 Hz, 1H), 5.19-4.98 (m, 1H), 4.97-4.60 (m, 3H), 2.92-2.77 (m, 1H), 2.72-2.57 (m, 2H), 2.06-1.97 (m, 1H), 1.59 (d, J=6.46 Hz, 2H), 1.50-1.35 (m, 3H), 1.09 (br d, J=6.46 Hz, 1H). LCMS (ESI) m/z 504 [M+H]⁺.

### Synthesis of Compound 24

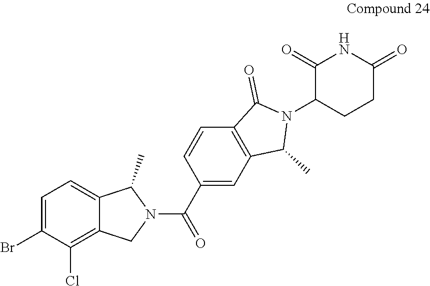 [SMILES: C[C@@H]1c2cc(C(=O)N3Cc4c(ccc(Br)c4Cl)[C@@H]3C)ccc2C(=O)N1C1CCC(=O)NC1=O]

### (3S)-3-[(3R)-5-[(1S)-5-bromo-4-chloro-1-methyl-isoindoline-2-carbonyl]-3-methyl-1-oxo-isoindolin-2-yl]piperidine-2,6-dione

Corresponding isoindoline was synthesized following Isoindoline Synthesis B and coupled to CRBN core 2 following Coupling Procedure B to afford (3 S)-3-[(3R)-5-[(1S)-5-bromo-4-chloro-1-methyl-isoindoline-2-carbonyl]-3-methyl-1-oxo-isoindolin-2-yl]piperidine-2,6-dione (14 mg, 19.7% yield) as a white solid. ¹H NMR (600 MHz, DMSO-d₆) δ ppm 10.97 (s, 1H), 7.98-7.88 (m, 1H), 7.78-7.65 (m, 3H), 7.38-7.08 (m, 1H), 5.57-5.25 (m, 1H), 5.10-4.38 (m, 4H), 2.92-2.81 (m, 1H), 2.70-2.52 (m, 2H), 2.05-2.00 (m, 1H), 1.56 (d, J=6.5 Hz, 2H), 1.58-1.31 (m, 3H), 1.06 (d, J=6.5 Hz, 1H). LCMS (ESI) m/z 530 [M+H]⁺.

### Synthesis of Compound 34

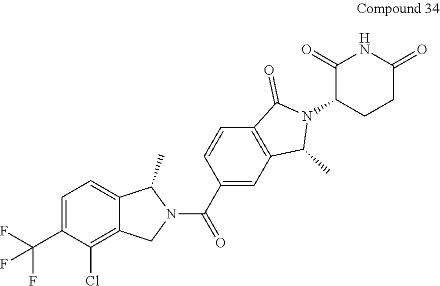 [SMILES: C[C@@H]1c2cc(C(=O)N3Cc4c(ccc(C(F)(F)F)c4Cl)[C@@H]3C)ccc2C(=O)N1[C@H]1CCC(=O)NC1=O]

### (3S)-3-[(3R)-5-[(1S)-4-chloro-1-methyl-5-(trifluoromethyl)isoindoline-2-carbonyl]-3-methyl-1-oxo-isoindolin-2-yl]piperidine-2,6-dione

Corresponding isoindoline was synthesized following Isoindoline Synthesis H and coupled to CRBN core 2 following Coupling Procedure B to afford (3 S)-3-[(3R)-5-[(1S)-4-chloro-1-methyl-5-(trifluoromethyl)isoindoline-2-carbonyl]-3-methyl-1-oxo-isoindolin-2-yl]piperidine-2,6-dione (29 mg, 72.5% yield) as a white solid. ¹H NMR (500 MHz, DMSO-d₆) δ ppm 10.97 (s, 1H), 7.91 (s, 1H), 7.86 (d, J=8.07 Hz, 1H), 7.80-7.75 (m, 1H), 7.75-7.66 (m, 1H), 7.65-7.45 (m, 1H), 5.71-5.37 (m, 1H), 5.15-4.87 (m, 1H), 4.87-4.69 (m, 2H), 4.60 (d, J=14.92 Hz, 1H), 2.97-2.74 (m, 1H), 2.74-2.57 (m, 2H), 2.06-1.98 (m, 1H), 1.60 (d, J=6.60 Hz, 2H), 1.46 (br d, J=6.60 Hz, 3H), 1.11 (br d, J=6.60 Hz, 1H). LCMS (ESI) m/z 520 [M+H]⁺.

### Synthesis of Compound 1

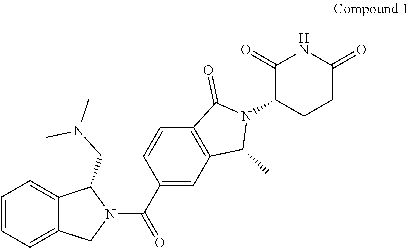 [SMILES: C[C@@H]1c2cc(C(=O)N3Cc4ccccc4[C@@H]3CN(C)C)ccc2C(=O)N1[C@H]1CCC(=O)NC1=O]

### (3S)-3-[(3R)-5-[(1R)-1-[(dimethylamino)methyl]isoindoline-2-carbonyl]-3-methyl-1-oxo-isoindolin-2-yl]piperidine-2,6-dione

Corresponding isoindoline was synthesized following Isoindoline Synthesis D and coupled to CRBN core 2 following Coupling Procedure C to afford a white powder (3S)-3-[(3R)-5-[(1R)-1-[(dimethylamino)methyl]isoindoline-2-carbonyl]-3-methyl-1-oxo-isoindolin-2-yl]piperidine-2,6-dione (12 mg, 70.2% yield). ¹H NMR (500 MHz, DMSO-d₆) δ ppm 10.97 (s, 1H), 7.86 (s, 1H), 7.80-7.72 (m, 1H), 7.67 (br d, J=8.0 Hz, 1H), 7.54-7.08 (m, 4H), 5.58-5.12 (m, 1H), 5.05-4.21 (m, 4H), 2.96-2.56 (m, 4H), 2.35-1.94 (m, 6H), 1.75 (br s, 2H), 1.46 (br d, J=6.6 Hz, 3H). LCMS (ESI) m/z 461 [M+H]⁺.

### Synthesis of Compound 29

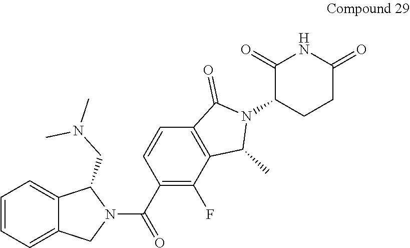 [SMILES: C[C@@H]1c2c(ccc(C(=O)N3Cc4ccccc4[C@@H]3CN(C)C)c2F)C(=O)N1[C@H]1CCC(=O)NC1=O]

### (3S)-3-[(3R)-5-[(1R)-1-[(dimethylamino)methyl]isoindoline-2-carbonyl]-3-methyl-1-oxo-isoindolin-2-yl]piperidine-2,6-dione

Corresponding isoindoline was synthesized following Isoindoline Synthesis D and coupled to CRBN core 5 following coupling procedure D. The coupling product was cyclized following Cyclization Procedure to afford (3S)-3-[(3R)-5-[(1R)-1-[(dimethylamino)methyl]isoindoline-2-carbonyl]-3-methyl-1-oxo-isoindolin-2-yl]piperidine-2,6-dione (12.1 mg, 70.2% yield) as a white powder. ¹H NMR (500 MHz, DMSO-d₆) δ ppm 11.00 (d, J=6.4 Hz, 1H), 7.78-7.55 (m, 2H), 7.52-7.40 (m, 1H), 7.38-7.11 (m, 3H), 5.46-4.35 (m, 5H), 2.96-2.54 (m, 5H), 2.22 (s, 4H), 2.03 (br dd, J=10.1, 5.1 Hz, 1H), 1.77 (s, 2H), 1.57-1.40 (m, 3H). LCMS (ESI) m/z 479 [M+H]⁺.

### Synthesis of Compound 8

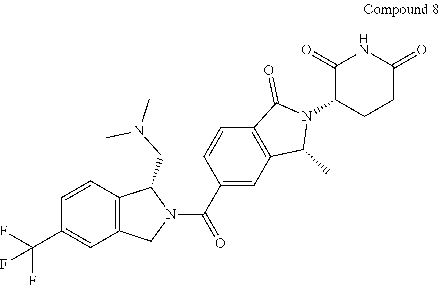 [SMILES: C[C@@H]1c2cc(C(=O)N3Cc4cc(C(F)(F)F)ccc4[C@@H]3CN(C)C)ccc2C(=O)N1[C@H]1CCC(=O)NC1=O]

### (3S)-3-[(3R)-5-[(1R)-1-[(dimethylamino)methyl]-5-(trifluoromethyl)isoindoline-2-carbonyl]-3-methyl-1-oxo-isoindolin-2-yl]piperidine-2,6-dione

Corresponding isoindoline was synthesized following Isoindoline Synthesis I and coupled to CRBN core 2 following Coupling Procedure C to afford (3S)-3-[(3R)-5-[(1R)-1-[(dimethylamino)methyl]-5-(trifluoromethyl)isoindoline-2-carbonyl]-3-methyl-1-oxo-isoindolin-2-yl]piperidine-2,6-dione (10 mg, 19.5% yield) as a white solid. ¹H NMR (600 MHz, DMSO-d₆) δ ppm 11.08-10.83 (m, 1H), 7.95-7.84 (m, 1H), 7.84-7.69 (m, 2H), 7.67 (br s, 2H), 7.63 (br s, 1H), 5.66-5.19 (m, 1H), 5.12-4.89 (m, 1H), 4.88-4.73 (m, 2H), 4.73-4.44 (m, 1H), 2.94-2.77 (m, 2H), 2.73-2.55 (m, 3H), 2.27 (br s, 4H), 2.08-1.96 (m, 1H), 1.77 (s, 2H), 1.46 (br d, J=6.6 Hz, 3H). LCMS (ESI) m/z 529 [M+H]⁺.

### Synthesis of Compound 3

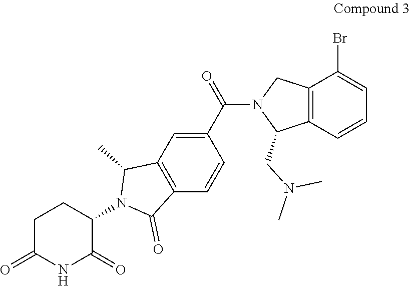 [SMILES: C[C@@H]1c2cc(C(=O)N3Cc4c(Br)cccc4[C@@H]3CN(C)C)ccc2C(=O)N1[C@H]1CCC(=O)NC1=O]

### (3S)-3-[(3R)-5-[(1R)-4-bromo-1-[(dimethylamino)methyl]isoindoline-2-carbonyl]-3-methyl-1-oxo-isoindolin-2-yl]piperidine-2,6-dione

To a solution of (3R)-2-[(3S)-2,6-dioxo-3-piperidyl]-3-methyl-1-oxo-isoindoline-5-carboxylic acid (90%, 50 mg, 0.149 mmol), 1-[(1R)-4-bromoisoindolin-1-yl]-N,N-dimethyl-methanamine dihydrochloride (54 mg, 0.164 mmol) and triethylamine (0.10 mL, 0.744 mmol) in DCM (0.7 mL) was added 2,4,6-tripropyl-1,3,5,2{5},4{5},6{5}-trioxatriphosphinane 2,4,6-trioxide (50% in EtOAc, 0.13 mL, 0.223 mmol). The reaction mixture was stirred under nitrogen for 1 h then EtOAc and saturated NaHCO₃ solution were added. The layers were separated and the aqueous layer was extracted twice with EtOAc. The combined organic layers were dried over sodium sulfate, filtered and concentrated under reduced pressure. The crude residue was purified by reversed phase flash chromatography (water/MeCN) to afford (3S)-3-[(3R)-5-[(1R)-4-bromo-1-[(dimethylamino)methyl]isoindoline-2-carbonyl]-3-methyl-1-oxo-isoindolin-2-yl]piperidine-2,6-dione (45.4 mg, 56.5% yield) as a white solid. ¹H NMR (500 MHz, DMSO-d₆) δ ppm 10.97 (br s, 1H), 7.88 (s, 1H), 7.82-7.73 (m, 1H), 7.70 (br d, J=7.2 Hz, 1H), 7.59-7.32 (m, 2H), 7.28 (t, J=7.4 Hz, 1H), 5.69-5.25 (m, 1H), 4.95-4.73 (m, 3H), 4.42 (br d, J=14.8 Hz, 1H), 2.96-2.71 (m, 1H), 2.73-2.56 (m, 3H), 2.26 (s, 4H), 2.25-2.09 (m, 1H), 2.10-1.95 (m, 1H), 1.76 (s, 2H), 1.46 (br d, J=6.5 Hz, 3H). LCMS (ESI) m/z 539[M+H]⁺.

### Synthesis of Compound 11

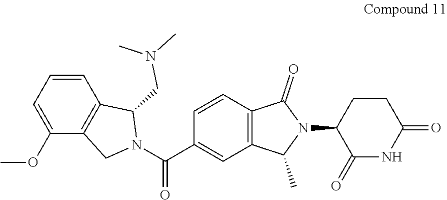 [SMILES: COc1cccc2c1CN(C(=O)c1ccc3c(c1)[C@@H](C)N([C@H]1CCC(=O)NC1=O)C3=O)[C@H]2CN(C)C]

### (3S)-3-[(3R)-5-[(1R)-1-[(dimethylamino)methyl]-4-methoxy-isoindoline-2-carbonyl]-3-methyl-1-oxo-isoindolin-2-yl]piperidine-2,6-dione

Corresponding isoindoline was synthesized following Isoindoline Synthesis D and coupled to CRBN core 2 following Coupling Procedure C to afford (3 S)-3-[(3R)-5-[(1R)-1-[(dimethylamino)methyl]-4-methoxy-isoindoline-2-carbonyl]-3-methyl-1-oxo-isoindolin-2-yl]piperidine-2,6-dione (25 mg, 42.8% yield) as a white solid. ¹H NMR (500 MHz, DMSO-d₆) δ ppm 10.97 (s, 1H), 7.85 (s, 1H), 7.88-7.71 (m, 1H), 7.67 (br d, J=7.8 Hz, 1H), 7.28 (t, J=7.9 Hz, 1H), 7.08-6.81 (m, 2H), 5.48-5.10 (m, 1H), 4.89-4.60 (m, 3H), 4.33 (d, J=14.3 Hz, 1H), 3.87-3.70 (m, 3H), 2.89-2.59 (m, 4H), 2.24 (s, 4H), 2.19-2.01 (m, 2H), 1.74 (s, 2H), 1.9-1.7 (m, 3H). LCMS (ESI) m/z 491 [M+H]⁺.

### Synthesis of Compound 14

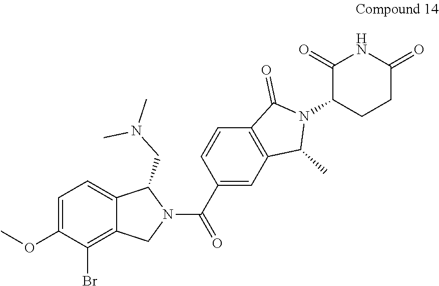 [SMILES: COc1ccc2c(c1Br)CN(C(=O)c1ccc3c(c1)[C@@H](C)N([C@H]1CCC(=O)NC1=O)C3=O)[C@H]2CN(C)C]

### (3S)-3-[(3R)-5-[(1R)-4-bromo-1-[(dimethylamino)methyl]-5-methoxy-isoindoline-2-carbonyl]-3-methyl-1-oxo-isoindolin-2-yl]piperidine-2,6-dione

Corresponding isoindoline was synthesized following Isoindoline Synthesis J and coupled to CRBN core 2 following Coupling Procedure C to afford (3S)-3-[(3R)-5-[(1R)-4-bromo-1-[(dimethylamino)methyl]-5-methoxy-isoindoline-2-carbonyl]-3-methyl-1-oxo-isoindolin-2-yl]piperidine-2,6-dione (77 mg, 45.4% yield) as a white powder. ¹H NMR (500 MHz, DMSO-d₆) δ ppm 11.15-10.74 (m, 1H), 7.87 (s, 1H), 7.81-7.73 (m, 1H), 7.73-7.62 (m, 1H), 7.46-7.25 (m, 1H), 7.13-6.94 (m, 1H), 5.71-5.10 (m, 1H), 5.01-4.25 (m, 4H), 3.98-3.68 (m, 3H), 2.96-2.56 (m, 4H), 1.76 (s, 8H), 1.46 (br d, J=6.4 Hz, 3H). LCMS (ESI) m/z 569 [M+H]⁺.

### Synthesis of Compound 28

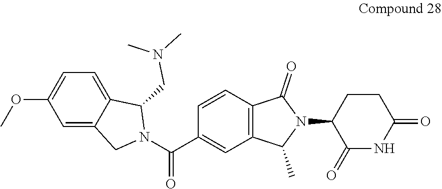 [SMILES: COc1ccc2c(c1)CN(C(=O)c1ccc3c(c1)[C@@H](C)N([C@H]1CCC(=O)NC1=O)C3=O)[C@H]2CN(C)C]

### 3S)-3-[(3R)-5-[(1R)-1-[(dimethylamino)methyl]-5-methoxy-isoindoline-2-carbonyl]-3-methyl-1-oxo-isoindolin-2-yl]piperidine-2,6-dione

Corresponding isoindoline was synthesized following Isoindoline Synthesis J and coupled to CRBN core 2 following Coupling Procedure C to afford 3S)-3-[(3R)-5-[(1R)-1-[(dimethylamino)methyl]-5-methoxy-isoindoline-2-carbonyl]-3-methyl-1-oxo-isoindolin-2-yl]piperidine-2,6-dione (25 mg, 17.1% yield) as a white solid. ¹H NMR (500 MHz, DMSO-d₆) δ ppm 11.00-10.91 (m, 1H), 7.90-7.82 (m, 1H), 7.80-7.61 (m, 2H), 7.38-7.14 (m, 1H), 7.04-6.76 (m, 2H), 5.46-5.06 (m, 1H), 4.97-4.32 (m, 4H), 3.66 (s, 3H), 2.90-2.55 (m, 4H), 2.34-1.65 (m, 8H), 1.55-1.36 (m, 3H). LCMS (ESI) m/z 491 [M+H]⁺.

### Synthesis of Compound 2

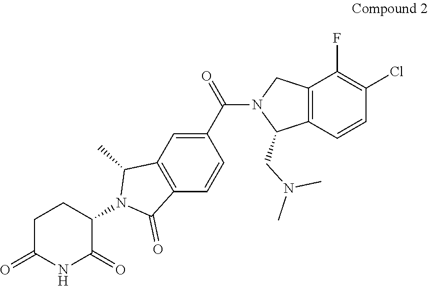 [SMILES: C[C@@H]1c2cc(C(=O)N3Cc4c(ccc(Cl)c4F)[C@@H]3CN(C)C)ccc2C(=O)N1[C@H]1CCC(=O)NC1=O]

### (3S)-3-[(3R)-5-[(1R)-5-chloro-1-[(dimethylamino)methyl]-4-fluoro-isoindoline-2-carbonyl]-3-methyl-1-oxo-isoindolin-2-yl]piperidine-2,6-dione

Corresponding isoindoline was synthesized following Isoindoline Synthesis D and coupled to CRBN core 2 following Coupling Procedure C to afford (3S)-3-[(3R)-5-[(1R)-5-chloro-1-[(dimethylamino)methyl]-4-fluoro-isoindoline-2-carbonyl]-3-methyl-1-oxo-isoindolin-2-yl]piperidine-2,6-dione (61 mg, 68.3% yield) as a white solid. ¹H NMR (600 MHz, DMSO-d₆) δ ppm 11.00-10.90 (m, 1H), 7.86 (br s, 1H), 7.80-7.72 (m, 1H), 7.68 (br d, J=7.48 Hz, 1H), 7.56-7.50 (m, 1H), 7.35-7.17 (m, 1H), 5.55-5.20 (m, 1H), 5.11-4.98 (m, 1H), 4.92-4.71 (m, 2H), 4.62 (br d, J=14.38 Hz, 1H), 2.90-2.75 (m, 2H), 2.71-2.56 (m, 3H), 2.25 (s, 4H), 2.07-1.97 (m, 1H), 1.76 (br s, 2H), 1.50-1.41 (m, 3H). LCMS (ESI) m/z 513 [M+H]⁺.

### Synthesis of Compound 17

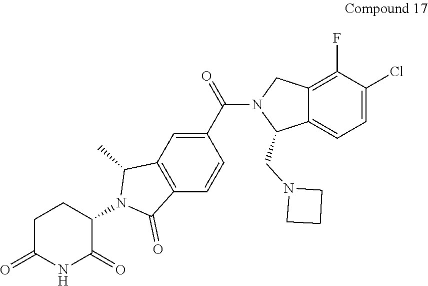 [SMILES: C[C@@H]1c2cc(C(=O)N3Cc4c(ccc(Cl)c4F)[C@@H]3CN3CCC3)ccc2C(=O)N1[C@H]1CCC(=O)NC1=O]

### (3S)-3-[(3R)-5-[(1R)-1-(azetidin-1-ylmethyl)-5-chloro-4-fluoro-isoindoline-2-carbonyl]-3-methyl-1-oxo-isoindolin-2-yl]piperidine-2,6-dione

Corresponding isoindoline was synthesized following Isoindoline Synthesis D, using corresponding secondary amine, then coupled to CRBN core 2 following Coupling Procedure C to afford (3S)-3-[(3R)-5-[(1R)-1-(azetidin-1-ylmethyl)-5-chloro-4-fluoro-isoindoline-2-carbonyl]-3-methyl-1-oxo-isoindolin-2-yl]piperidine-2,6-dione (32 mg, 40.9% yield) as a white solid. ¹H NMR (500 MHz, DMSO-d₆) δ ppm 10.97 (s, 1H), 7.92-7.82 (m, 1H), 7.78 (d, J=7.8 Hz, 1H), 7.67 (dd, J=7.8, 1.0 Hz, 1H), 7.54 (t, J=7.5 Hz, 1H), 7.39-7.16 (m, 1H), 5.47-5.10 (m, 1H), 5.09-4.93 (m, 1H), 4.89-4.55 (m, 3H), 3.20 (q, J=6.4 Hz, 1H), 3.12-2.97 (m, 2H), 2.97-2.73 (m, 2H), 2.71-2.57 (m, 3H), 2.43-2.00 (m, 2H), 1.99-1.71 (m, 2H), 1.45 (d, J=6.8 Hz, 3H). LCMS (ESI) m/z 525 [M+H]⁺.

### Synthesis of Compound 15

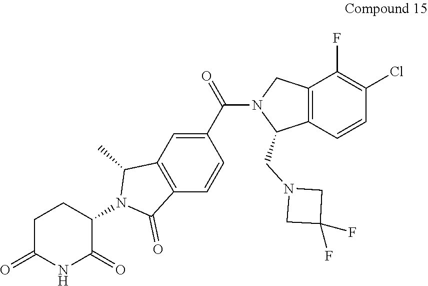 [SMILES: C[C@@H]1c2cc(C(=O)N3Cc4c(ccc(Cl)c4F)[C@@H]3CN3CC(F)(F)C3)ccc2C(=O)N1[C@H]1CCC(=O)NC1=O]

### (3S)-3-[(3R)-5-[(1R)-5-chloro-1-[(3,3-difluoroazetidin-1-yl)methyl]-4-fluoro-isoindoline-2-carbonyl]-3-methyl-1-oxo-isoindolin-2-yl]piperidine-2,6-dione

Corresponding isoindoline was synthesized following Isoindoline Synthesis D, using corresponding difluoroazetidine, then coupled to CRBN core 2 following Coupling Procedure C to afford (3S)-3-[(3R)-5-[(1R)-5-chloro-1-[(3,3-difluoroazetidin-1-yl)methyl]-4-fluoro-isoindoline-2-carbonyl]-3-methyl-1-oxo-isoindolin-2-yl]piperidine-2,6-dione (32 mg, 33.5% yield) as a white solid. ¹H NMR (600 MHz, DMSO-d₆) δ ppm 10.96 (s, 1H), 7.95-7.83 (m, 1H), 7.77 (d, J=7.9 Hz, 1H), 7.70 (d, J=8.1 Hz, 1H), 7.59-7.50 (m, 1H), 7.32 (d, J=8.2 Hz, 1H), 5.47 (br s, 1H), 5.14-4.49 (m, 4H), 3.77-3.47 (m, 3H), 3.30-2.97 (m, 3H), 2.92-2.78 (m, 1H), 2.72-2.56 (m, 2H), 2.06-1.95 (m, 1H), 1.53-1.36 (m, 3H). LCMS (ESI) m/z 561 [M+H]⁺.

### Synthesis of Compound 4

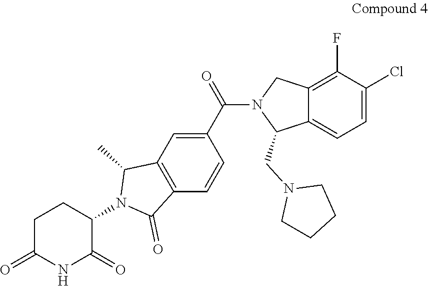 [SMILES: C[C@@H]1c2cc(C(=O)N3Cc4c(ccc(Cl)c4F)[C@@H]3CN3CCCC3)ccc2C(=O)N1[C@H]1CCC(=O)NC1=O]

### (3S)-3-[(3R)-5-[(1R)-5-chloro-4-fluoro-1-(pyrrolidin-1-ylmethyl)isoindoline-2-carbonyl]-3-methyl-1-oxo-isoindolin-2-yl]piperidine-2,6-dione

Corresponding isoindoline was synthesized following Isoindoline Synthesis D, using corresponding pyrolidine, then coupled to CRBN core 2 following Coupling Procedure C to afford (3S)-3-[(3R)-5-[(1R)-5-chloro-4-fluoro-1-(pyrrolidin-1-ylmethyl)isoindoline-2-carbonyl]-3-methyl-1-oxo-isoindolin-2-yl]piperidine-2,6-dione (28 mg, 50.4% yield) as a white solid. ¹H NMR (600 MHz, DMSO-d₆) δ ppm 10.98-10.94 (m, 1H), 7.92-7.82 (m, 1H), 7.76 (br s, 1H), 7.71-7.62 (m, 1H), 7.58-7.49 (m, 1H), 7.38-7.12 (m, 1H), 5.63-5.18 (m, 1H), 5.13-4.45 (m, 4H), 3.02-2.90 (m, 1H), 2.89-2.54 (m, 5H), 2.23-2.09 (m, 1H), 2.06-1.98 (m, 1H), 1.94-1.84 (m, 1H), 1.76-1.62 (m, 3H), 1.54-1.38 (m, 5H). LCMS (ESI) m/z 539 [M+H]⁺.

### Synthesis of Compound 6

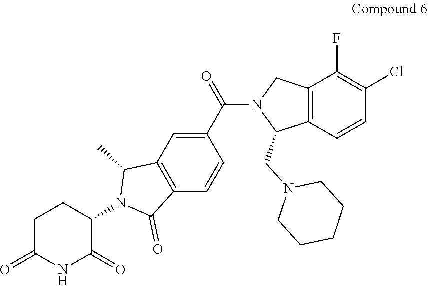 [SMILES: C[C@@H]1c2cc(C(=O)N3Cc4c(ccc(Cl)c4F)[C@@H]3CN3CCCCC3)ccc2C(=O)N1[C@H]1CCC(=O)NC1=O]

### (3S)-3-[(3R)-5-[(1R)-5-chloro-4-fluoro-1-(1-piperidylmethyl)isoindoline-2-carbonyl]-3-methyl-1-oxo-isoindolin-2-yl]piperidine-2,6-dione

Corresponding isoindoline was synthesized following Isoindoline Synthesis D, using corresponding piperidine, then coupled to CRBN core 2 following Coupling Procedure C to afford (3S)-3-[(3R)-5-[(1R)-5-chloro-4-fluoro-1-(1-piperidylmethyl)isoindoline-2-carbonyl]-3-methyl-1-oxo-isoindolin-2-yl]piperidine-2,6-dione (31 mg, 56.7% yield) as a white solid. ¹H NMR (500 MHz, DMSO-d₆) δ ppm 10.97 (s, 1H), 7.98-7.80 (m, 1H), 7.80-7.74 (m, 1H), 7.68 (m, J=7.4 Hz, 1H), 7.54 (t, J=7.6 Hz, 1H), 7.43-7.20 (m, 1H), 5.60-5.18 (m, 1H), 5.13-4.90 (m, 1H), 4.89-4.54 (m, 3H), 2.90-2.53 (m, 5H), 2.41-1.70 (m, 5H), 1.60-1.47 (m, 2H), 1.45 (d, J=6.8 Hz, 3H), 1.42-1.15 (m, 4H). LCMS (ESI) m/z 553 [M+H]⁺.

### Synthesis of Compound 12

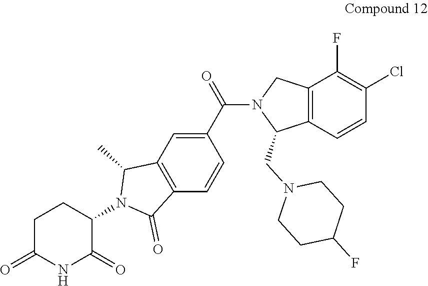 [SMILES: C[C@@H]1c2cc(C(=O)N3Cc4c(ccc(Cl)c4F)[C@@H]3CN3CCC(F)CC3)ccc2C(=O)N1[C@H]1CCC(=O)NC1=O]

### (3S)-3-[(3R)-5-[(1R)-5-chloro-4-fluoro-1-[(4-fluoro-1-piperidyl)methyl]isoindoline-2-carbonyl]-3-methyl-1-oxo-isoindolin-2-yl]piperidine-2,6-dione

Corresponding isoindoline was synthesized following Isoindoline Synthesis D, using corresponding piperidine, then coupled to CRBN core 2 following Coupling Procedure C to afford (3S)-3-[(3R)-5-[(1R)-5-chloro-4-fluoro-1-[(4-fluoro-1-piperidyl)methyl]isoindoline-2-carbonyl]-3-methyl-1-oxo-isoindolin-2-yl]piperidine-2,6-dione (61 mg, 60.5% yield) as a white solid. ¹H NMR (600 MHz, DMSO-d₆) δ ppm 10.96 (s, 1H), 8.01-7.81 (m, 1H), 7.80-7.73 (m, 1H), 7.68 (br d, J=7.3 Hz, 1H), 7.53 (t, J=7.5 Hz, 1H), 7.47-7.06 (m, 1H), 5.50 (br dd, J=3.3, 1.7 Hz, 1H), 4.79 (br d, J=4.1 Hz, 5H), 3.00-2.42 (m, 6H), 2.36-2.11 (m, 2H), 2.08-1.25 (m, 9H). LCMS (ESI) m/z 571 [M+H]⁺.

### Synthesis of Compound 10

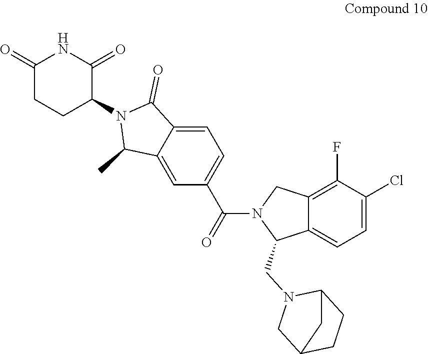 [SMILES: C[C@@H]1c2cc(C(=O)N3Cc4c(ccc(Cl)c4F)[C@@H]3CN3CC4CCC3C4)ccc2C(=O)N1[C@H]1CCC(=O)NC1=O]

### (3S)-3-[(3R)-5-[(1R)-1-(2-azabicyclo[2.2.1]heptan-2-ylmethyl)-5-chloro-4-fluoro-isoindoline-2-carbonyl]-3-methyl-1-oxo-isoindolin-2-yl]piperidine-2,6-dione

Corresponding isoindoline was synthesized following Isoindoline Synthesis D, using corresponding secondary amine, then coupled to CRBN core 2 following Coupling Procedure C to afford (3S)-3-[(3R)-5-[(1R)-1-(2-azabicyclo[2.2.1]heptan-2-ylmethyl)-5-chloro-4-fluoro-isoindoline-2-carbonyl]-3-methyl-1-oxo-isoindolin-2-yl]piperidine-2,6-dione (34 mg, 62.0% yield) as a white solid. ¹H NMR (600 MHz, DMSO-d₆) δ ppm 11.45-10.64 (m, 1H), 7.94-7.81 (m, 1H), 7.79-7.60 (m, 2H), 7.52 (t, J=7.2 Hz, 1H), 7.40-7.10 (m, 1H), 5.38 (br dd, J=3.5, 2.3 Hz, 1H), 5.05-4.45 (m, 4H), 3.14-2.77 (m, 3H), 2.75-2.53 (m, 3H), 2.47-2.18 (m, 2H), 2.18-1.95 (m, 2H), 1.87-1.47 (m, 2H), 1.44 (dd, J=6.5, 2.1 Hz, 3H), 1.41-1.24 (m, 2H), 1.19-0.74 (m, 2H). LCMS (ESI) m/z 565 [M+H]⁺.

### Synthesis of Compound 7

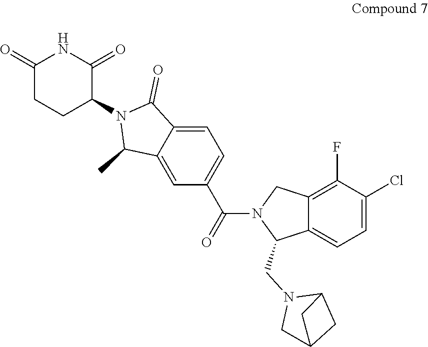 [SMILES: C[C@@H]1c2cc(C(=O)N3Cc4c(ccc(Cl)c4F)[C@@H]3CN3CC4CC3C4)ccc2C(=O)N1[C@H]1CCC(=O)NC1=O]

### (3S)-3-[(3R)-5-[(1R)-1-(2-azabicyclo[2.1.1]hexan-2-ylmethyl)-5-chloro-4-fluoro-isoindoline-2-carbonyl]-3-methyl-1-oxo-isoindolin-2-yl]piperidine-2,6-dione

Corresponding isoindoline was synthesized following Isoindoline Synthesis D, using corresponding secondary amine, then coupled to CRBN core 2 following Coupling Procedure C to afford (3S)-3-[(3R)-5-[(1R)-1-(2-azabicyclo[2.1.1]hexan-2-ylmethyl)-5-chloro-4-fluoro-isoindoline-2-carbonyl]-3-methyl-1-oxo-isoindolin-2-yl]piperidine-2,6-dione (31.1 mg, 37.0% yield) as a white solid. ¹H NMR (600 MHz, DMSO-d₆) δ ppm 11.11-10.74 (m, 1H), 7.95-7.82 (m, 1H), 7.81-7.72 (m, 1H), 7.71-7.63 (m, 1H), 7.53 (t, J=7.6 Hz, 1H), 7.48-7.18 (m, 1H), 5.47 (br d, J=5.0 Hz, 1H), 5.12-4.57 (m, 4H), 3.28-2.55 (m, 7H), 2.48-2.39 (m, 1H), 2.29-1.77 (m, 2H), 1.69-1.25 (m, 6H), 1.07-0.86 (m, 1H). LCMS (ESI) m/z 551 [M+H]⁺.

### Synthesis of Compound 16

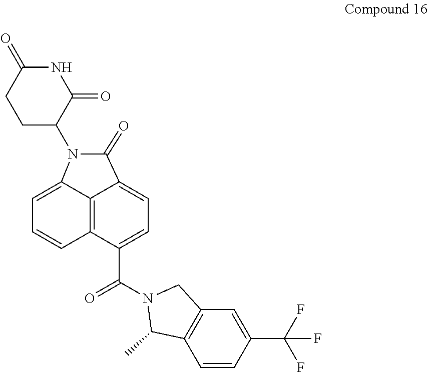 [SMILES: C[C@H]1c2ccc(C(F)(F)F)cc2CN1C(=O)c1ccc2c3c(cccc13)N(C1CCC(=O)NC1=O)C2=O]

### 3-[5-[(1S)-1-methyl-5-(trifluoromethyl)isoindoline-2-carbonyl]-2-oxo-benzo[cd]indol-1-yl]piperidine-2,6-dione

Corresponding isoindoline was synthesized following Isoindoline Synthesis A and coupled to CRBN core 6 following Coupling Procedure B to afford 3-[5-[(1S)-1-methyl-5-(trifluoromethyl)isoindoline-2-carbonyl]-2-oxo-benzo[cd]indol-1-yl]piperidine-2,6-dione (26 mg, 32.9% yield) as a yellow powder. ¹H NMR (600 MHz, DMSO-d₆): δ ppm 11.15 (s, 1H), 8.21-8.10 (m, 1H), 7.93 (d, J=7.0 Hz, 1H), 7.71-7.60 (m, 2H), 7.59-7.51 (m, 3H), 7.31-7.22 (m, 1H), 5.64 (q, J=6.5 Hz, 1H), 5.50-5.40 (m, 1H), 4.67 (br d, J=15.0 Hz, 1H), 4.45 (d, J=15.1 Hz, 1H), 3.01-2.92 (m, 1H), 2.79-2.71 (m, 1H), 2.70-2.62 (m, 1H), 2.21-2.12 (m, 1H), 1.71 (d, J=6.5 Hz, 3H). LCMS (ESI) m/z 508 [M+H]⁺.

### Synthesis of Compound 5

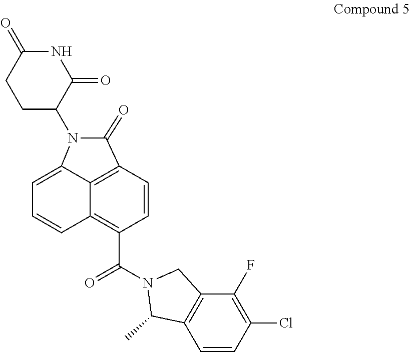 [SMILES: C[C@H]1c2ccc(Cl)c(F)c2CN1C(=O)c1ccc2c3c(cccc13)N(C1CCC(=O)NC1=O)C2=O]

### 3-[5-[(1S)-5-chloro-4-fluoro-1-methyl-isoindoline-2-carbonyl]-2-oxo-benzo[cd]indol-1-yl]piperidine-2,6-dione

Corresponding isoindoline was synthesized following Isoindoline Synthesis C and coupled to the corresponding carboxylic acid following Coupling Procedure B to afford 3-[5-[(1S)-5-chloro-4-fluoro-1-methyl-isoindoline-2-carbonyl]-2-oxo-benzo[cd]indol-1-yl]piperidine-2,6-dione (38 mg, 49.8% yield) as a light yellow powder. ¹H NMR (500 MHz, DMSO-d₆) δ ppm 11.15 (s, 1H), 8.23-8.15 (m, 1H), 7.99-7.94 (m, 1H), 7.64-7.49 (m, 3H), 7.33 (d, J=8.13 Hz, 1H), 7.23 (d, J=6.88 Hz, 1H), 5.65-5.57 (m, 1H), 5.54-5.44 (m, 1H), 4.81-4.71 (m, 1H), 4.52 (d, J=14.88 Hz, 1H), 3.03-2.90 (m, 1H), 2.86-2.72 (m, 1H), 2.71-2.62 (m, 1H), 2.18-2.09 (m, 1H), 1.69 (d, J=6.38 Hz, 3H). LCMS (ESI) m/z 492 [M+H]⁺.

### Example 17: ARNT Degradation

Compounds of Formula (I) were tested for ARNT degradation in a HiBiT system. A HEK293 ARNT-HiBiT Knock-In cell line was generated using CRISPR/Cas9 technology. Briefly, HEK293 cells were electroporated with the Neon system with an sgRNA targeting the C-terminus of the ARNT gene near the stop codon (CCTTTTCAGAATAGAACTAT (SEQ ID NO: 1); chr 1: 150,812,014-150,812,034) and the HiBiT donor DNA template (80 nucleotides homology arms upstream and downstream of the insert site, with a GSGS (SEQ ID NO: 2) linker). A homozygous clone was selected after Sanger sequencing, and genetic edition was further validated using Nano-Glo HiBiT blotting system. HEK293 ARNT-HiBiT KI cells were cultured in DMEM+10% FBS at 37° C. and 5% CO₂. For the assay, cells were seeded at 10,000 cells/well in white 384 plates (Corning #3570) in 22.5 μl of culture medium without phenol red. Test compounds were spotted in 384-well plates by an acoustic dispenser (10 concentrations, semi-log dilution, in 100% DMSO). Before treatment, compounds were diluted 1:100 or 1:1,000 in culture medium without phenol red, and cells were treated with 2.5 μl of diluted compounds 18h after seeding (final top concentration: 3 or 0.3 μM). After 4h of treatment, plates were equilibrated at room temperature for 30 min, and 25 μl of NANO-GLO® HiBiT Lytic reagent were added per well. Luminescence was measured on a plate reader 15 min after incubation with the detection reagent. Data were analyzed using the Genedata Screener Software version 19, and normalization was performed using the DMSO treated wells as Neutral Control and medium only (without cells) wells as Scale Reference. For curve fitting, the smart fit condensing method with a 4-parameters Hill model was used and the half-maximal degradation concentration (DC₅₀) was calculated. Results are reported in Table 2.

TABLE 2

| | | Iso- | Coupl- | |
|---|---|---|---|---|
| | | indoline | ing | HiBiT |
| Cmpd. | | syn- | proce- | DC₅₀ |
| No. | Compound Structure | thesis | dure | (nM) |
| 38 | 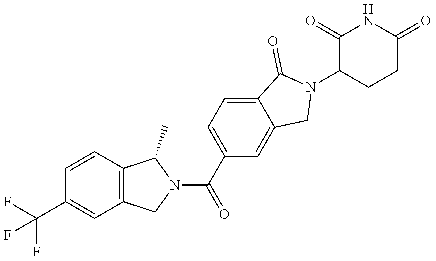 [SMILES: C[C@H]1c2ccc(C(F)(F)F)cc2CN1C(=O)c1ccc2c(c1)CN(C1CCC(=O)NC1=O)C2=O] | A | A | 7.6 |
| 31 | 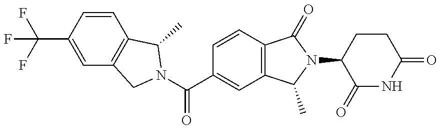 [SMILES: C[C@@H]1c2cc(C(=O)N3Cc4cc(C(F)(F)F)ccc4[C@@H]3C)ccc2C(=O)N1[C@H]1CCC(=O)NC1=O] | A | B | 2.2 |
| 21 | 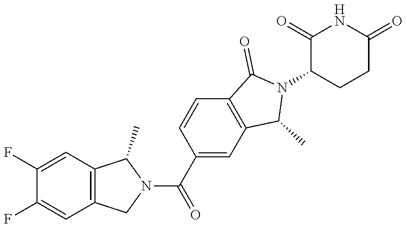 [SMILES: C[C@@H]1c2cc(C(=O)N3Cc4cc(F)c(F)cc4[C@@H]3C)ccc2C(=O)N1[C@H]1CCC(=O)NC1=O] | A | D | 1.3 |
| 26 | 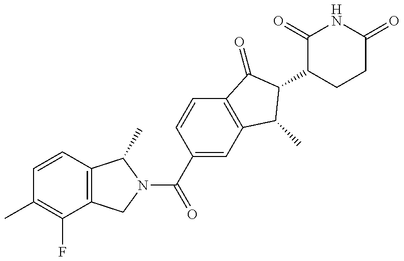 [SMILES: Cc1ccc2c(c1F)CN(C(=O)c1ccc3c(c1)[C@@H](C)[C@@H](C1CCC(=O)NC1=O)C3=O)[C@H]2C] | D | D | 1.7 |
| 27 | 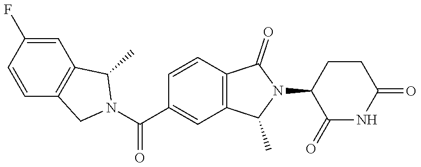 [SMILES: C[C@@H]1c2cc(C(=O)N3Cc4ccc(F)cc4[C@@H]3C)ccc2C(=O)N1[C@H]1CCC(=O)NC1=O] | A | D | 1.7 |
| 36 | 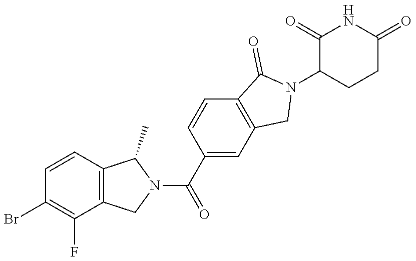 [SMILES: C[C@H]1c2ccc(Br)c(F)c2CN1C(=O)c1ccc2c(c1)CN(C1CCC(=O)NC1=O)C2=O] | B | A | 3.4 |
| 19 | 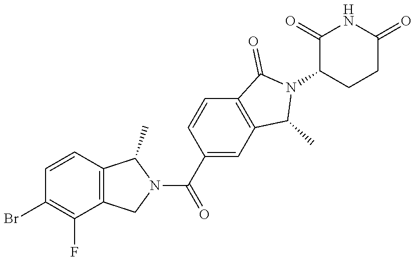 [SMILES: C[C@@H]1c2cc(C(=O)N3Cc4c(ccc(Br)c4F)[C@@H]3C)ccc2C(=O)N1[C@H]1CCC(=O)NC1=O] | B | B | 1.2 |
| 30 | 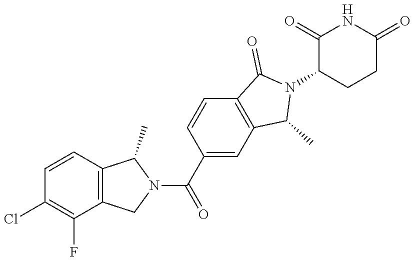 [SMILES: C[C@@H]1c2cc(C(=O)N3Cc4c(ccc(Cl)c4F)[C@@H]3C)ccc2C(=O)N1[C@H]1CCC(=O)NC1=O] | D | B | 1.9 |
| 13 | 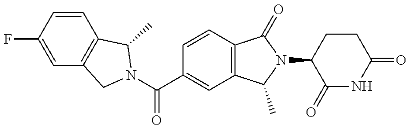 [SMILES: C[C@@H]1c2cc(C(=O)N3Cc4cc(F)ccc4[C@@H]3C)ccc2C(=O)N1[C@H]1CCC(=O)NC1=O] | A | D | 0.78 |
| 9 | 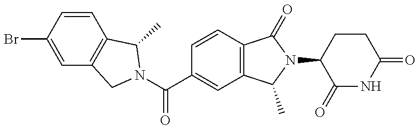 [SMILES: C[C@@H]1c2cc(C(=O)N3Cc4cc(Br)ccc4[C@@H]3C)ccc2C(=O)N1[C@H]1CCC(=O)NC1=O] | A | B | 0.66 |
| 22 | 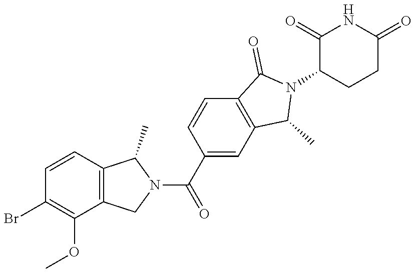 [SMILES: COc1c(Br)ccc2c1CN(C(=O)c1ccc3c(c1)[C@@H](C)N([C@H]1CCC(=O)NC1=O)C3=O)[C@H]2C] | E | B | 1.3 |
| 37 | 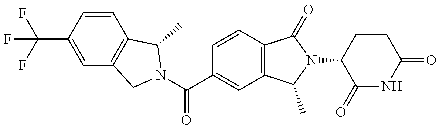 [SMILES: C[C@@H]1c2cc(C(=O)N3Cc4cc(C(F)(F)F)ccc4[C@@H]3C)ccc2C(=O)N1[C@@H]1CCC(=O)NC1=O] | A | D | 6.5 |
| 25 | 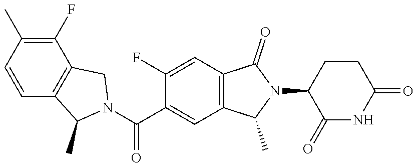 [SMILES: Cc1ccc2c(c1F)CN(C(=O)c1cc3c(cc1F)C(=O)N([C@H]1CCC(=O)NC1=O)[C@@H]3C)[C@H]2C] | D | B | 1.6 |
| 20 | 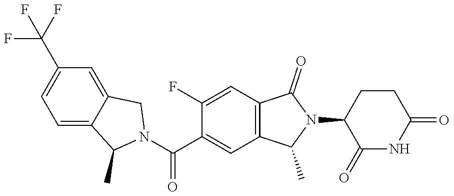 [SMILES: C[C@@H]1c2cc(C(=O)N3Cc4cc(C(F)(F)F)ccc4[C@@H]3C)c(F)cc2C(=O)N1[C@H]1CCC(=O)NC1=O] | A | B | 1.3 |
| 35 | 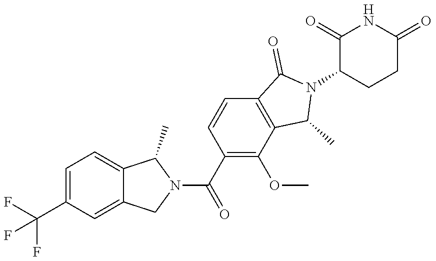 [SMILES: COc1c(C(=O)N2Cc3cc(C(F)(F)F)ccc3[C@@H]2C)ccc2c1[C@@H](C)N([C@H]1CCC(=O)NC1=O)C2=O] | A | B | 3.3 |
| 33 | 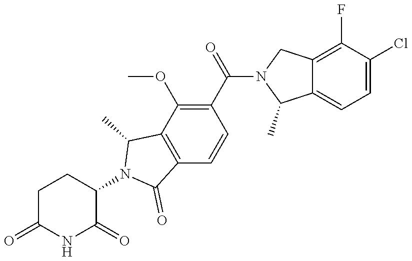 [SMILES: COc1c(C(=O)N2Cc3c(ccc(Cl)c3F)[C@@H]2C)ccc2c1[C@@H](C)N([C@H]1CCC(=O)NC1=O)C2=O] | C | D | 3.0 |
| 18 | 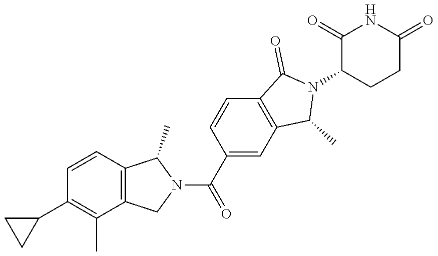 [SMILES: Cc1c(C2CC2)ccc2c1CN(C(=O)c1ccc3c(c1)[C@@H](C)N([C@H]1CCC(=O)NC1=O)C3=O)[C@H]2C] | F | B | 1.2 |
| 23 | 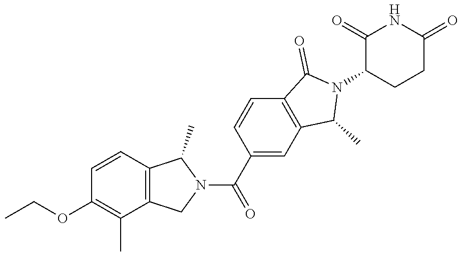 [SMILES: CCOc1ccc2c(c1C)CN(C(=O)c1ccc3c(c1)[C@@H](C)N([C@H]1CCC(=O)NC1=O)C3=O)[C@H]2C] | G | B | 1.3 |
| 32 | 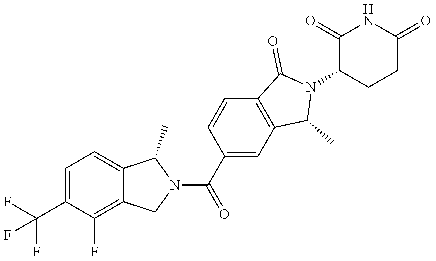 [SMILES: C[C@@H]1c2cc(C(=O)N3Cc4c(ccc(C(F)(F)F)c4F)[C@@H]3C)ccc2C(=O)N1[C@H]1CCC(=O)NC1=O] | A | B | 2.3 |
| 24 | 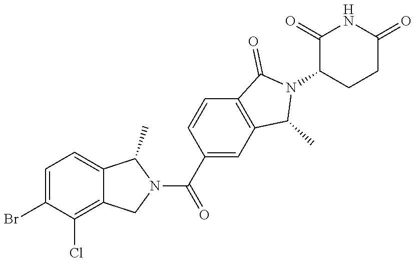 [SMILES: C[C@@H]1c2cc(C(=O)N3Cc4c(ccc(Br)c4Cl)[C@@H]3C)ccc2C(=O)N1[C@H]1CCC(=O)NC1=O] | B | B | 1.3 |
| 34 | 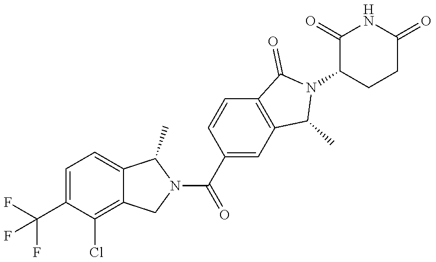 [SMILES: C[C@@H]1c2cc(C(=O)N3Cc4c(ccc(C(F)(F)F)c4Cl)[C@@H]3C)ccc2C(=O)N1[C@H]1CCC(=O)NC1=O] | H | B | 3.1 |
| 1 | 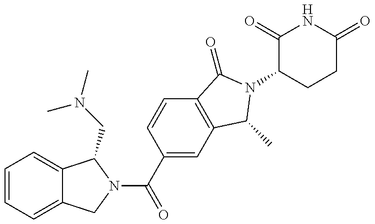 [SMILES: C[C@@H]1c2cc(C(=O)N3Cc4ccccc4[C@@H]3CN(C)C)ccc2C(=O)N1[C@H]1CCC(=O)NC1=O] | D | C | 0.29 |
| 29 | 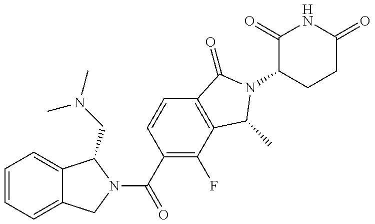 [SMILES: C[C@@H]1c2c(ccc(C(=O)N3Cc4ccccc4[C@@H]3CN(C)C)c2F)C(=O)N1[C@H]1CCC(=O)NC1=O] | D | D | 1.8 |
| 8 | 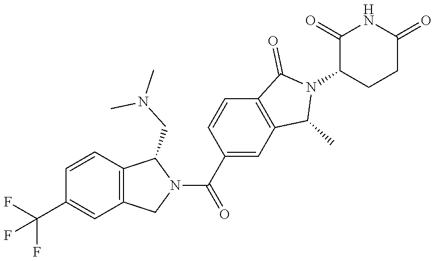 [SMILES: C[C@@H]1c2cc(C(=O)N3Cc4cc(C(F)(F)F)ccc4[C@@H]3CN(C)C)ccc2C(=O)N1[C@H]1CCC(=O)NC1=O] | I | C | 0.61 |
| 3 | 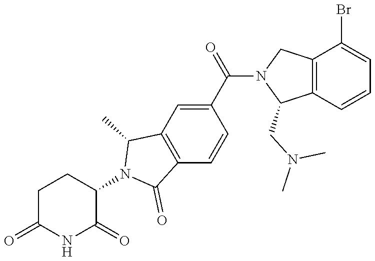 [SMILES: C[C@@H]1c2cc(C(=O)N3Cc4c(Br)cccc4[C@@H]3CN(C)C)ccc2C(=O)N1[C@H]1CCC(=O)NC1=O] | D | C | 0.39 |
| 11 | 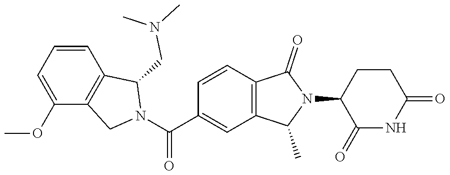 [SMILES: COc1cccc2c1CN(C(=O)c1ccc3c(c1)[C@@H](C)N([C@H]1CCC(=O)NC1=O)C3=O)[C@H]2CN(C)C] | D | C | 0.72 |
| 14 | 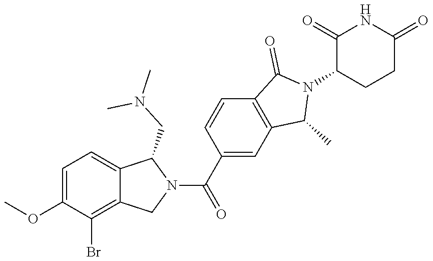 [SMILES: COc1ccc2c(c1Br)CN(C(=O)c1ccc3c(c1)[C@@H](C)N([C@H]1CCC(=O)NC1=O)C3=O)[C@H]2CN(C)C] | J | C | 0.95 |
| 28 | 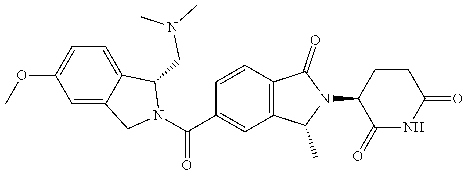 [SMILES: COc1ccc2c(c1)CN(C(=O)c1ccc3c(c1)[C@@H](C)N([C@H]1CCC(=O)NC1=O)C3=O)[C@H]2CN(C)C] | J | C | 1.7 |
| 2 | 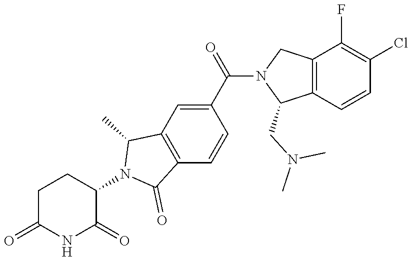 [SMILES: C[C@@H]1c2cc(C(=O)N3Cc4c(ccc(Cl)c4F)[C@@H]3CN(C)C)ccc2C(=O)N1[C@H]1CCC(=O)NC1=O] | D | C | 0.33 |
| 17 | 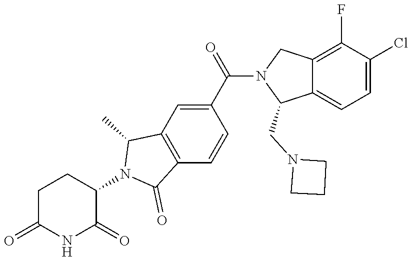 [SMILES: C[C@@H]1c2cc(C(=O)N3Cc4c(ccc(Cl)c4F)[C@@H]3CN3CCC3)ccc2C(=O)N1[C@H]1CCC(=O)NC1=O] | D | C | 1.2 |
| 15 | 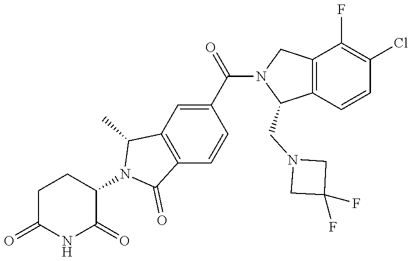 [SMILES: C[C@@H]1c2cc(C(=O)N3Cc4c(ccc(Cl)c4F)[C@@H]3CN3CC(F)(F)C3)ccc2C(=O)N1[C@H]1CCC(=O)NC1=O] | D | C | 1.0 |
| 4 | 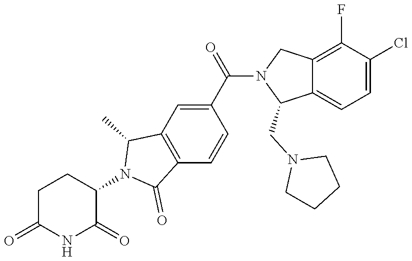 [SMILES: C[C@@H]1c2cc(C(=O)N3Cc4c(ccc(Cl)c4F)[C@@H]3CN3CCCC3)ccc2C(=O)N1[C@H]1CCC(=O)NC1=O] | D | C | 0.42 |
| 6 | 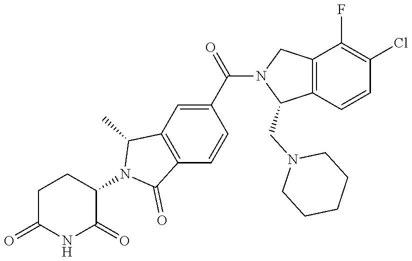 [SMILES: C[C@@H]1c2cc(C(=O)N3Cc4c(ccc(Cl)c4F)[C@@H]3CN3CCCCC3)ccc2C(=O)N1[C@H]1CCC(=O)NC1=O] | D | C | 0.46 |
| 12 | 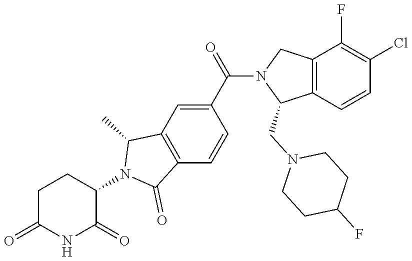 [SMILES: C[C@@H]1c2cc(C(=O)N3Cc4c(ccc(Cl)c4F)[C@@H]3CN3CCC(F)CC3)ccc2C(=O)N1[C@H]1CCC(=O)NC1=O] | D | C | 0.73 |
| 10 | 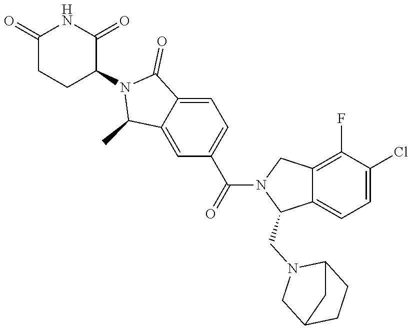 [SMILES: C[C@@H]1c2cc(C(=O)N3Cc4c(ccc(Cl)c4F)[C@@H]3CN3CC4CCC3C4)ccc2C(=O)N1[C@H]1CCC(=O)NC1=O] | D | C | 0.71 |
| 7 | 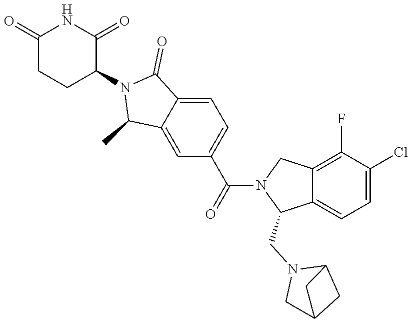 [SMILES: C[C@@H]1c2cc(C(=O)N3Cc4c(ccc(Cl)c4F)[C@@H]3CN3CC4CC3C4)ccc2C(=O)N1[C@H]1CCC(=O)NC1=O] | D | C | 0.48 |
| 16 | 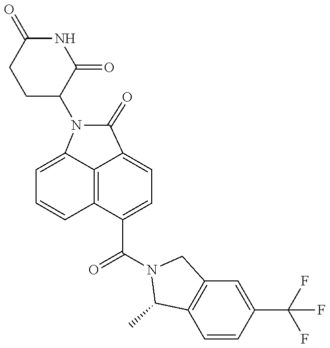 [SMILES: C[C@H]1c2ccc(C(F)(F)F)cc2CN1C(=O)c1ccc2c3c(cccc13)N(C1CCC(=O)NC1=O)C2=O] | A | B | 1.0 |
| 5 | 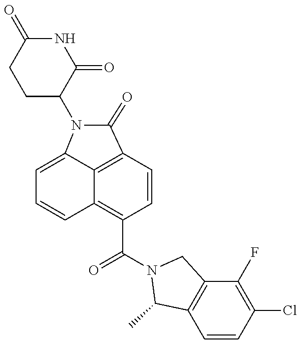 [SMILES: C[C@H]1c2ccc(Cl)c(F)c2CN1C(=O)c1ccc2c3c(cccc13)N(C1CCC(=O)NC1=O)C2=O] | A | B | 0.42 |

A number of references have been cited herein, and their disclosures are incorporated herein by reference in their entireties.

1. A compound of the following structural formula: 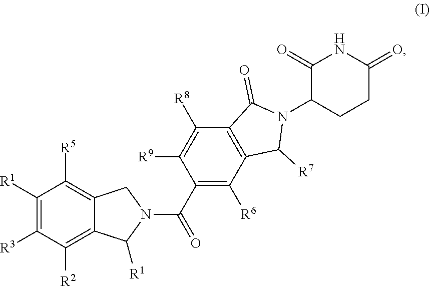
or a pharmaceutically acceptable salt, tautomer, isotopologue, or stereoisomer thereof, wherein:
R¹ is (C₁-C₃)alkyl or —CH₂N(R¹⁰)₂;
each R¹⁰ is independently (C₁-C₃)alkyl or two R¹⁰, together with the nitrogen to which they are attached, form a four- to eight-membered heterocyclyl optionally substituted with (R¹¹)ₓ;
each R¹¹ is independently halo, (C₁-C₃)alkyl, or halo(C₁-C₃)alkyl;
x is 1, 2, 3, 4, or 5;
R² is H, halo, (C₁-C₃)alkyl, halo(C₁-C₃)alkyl, —O—(C₁-C₃)alkyl, or (C₃-C₅)cycloalkyl;
R³ is H, halo, (C₁-C₃)alkyl, halo(C₁-C₃)alkyl, —O—(C₁-C₃)alkyl, or (C₃-C₅)cycloalkyl;
R⁴ is H, halo, (C₁-C₃)alkyl, halo(C₁-C₃)alkyl, —O—(C₁-C₃)alkyl, or (C₃-C₅)cycloalkyl;
R⁵ is H, halo, (C₁-C₃)alkyl, halo(C₁-C₃)alkyl, —O—(C₁-C₃)alkyl, or (C₃-C₅)cycloalkyl;
R⁶ is H, halo, (C₁-C₃)alkyl, halo(C₁-C₃)alkyl, or —O—(C₁-C₃)alkyl; and
R⁷ is H or (C₁-C₃)alkyl; or
R⁶ and R⁷, taken together with their intervening atoms, form a 5-7-membered cycle; and
R⁸ and R⁹ are each independently H or halo.

2-11. (canceled)

12. The compound of claim 1, wherein R¹ is —CH₃, —CH₂N(CH₃)₂,

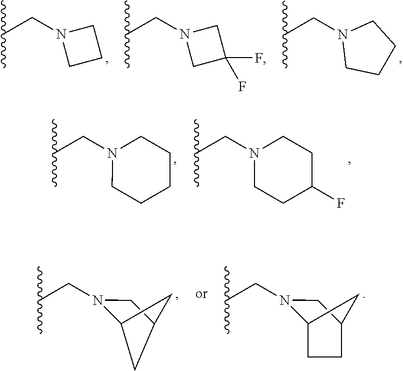

13. (canceled)

14. The compound of claim 1, wherein R² is H or F.

15. (canceled)

16. The compound of claim 1, wherein R³ is H or halo.

17-18. (canceled)

19. The compound of claim 1, wherein R⁴ is H, F, Cl, Br, methyl, trifluoromethyl, methoxy, ethoxy, or cyclopropyl.

20-22. (canceled)

23. The compound of claim 1, wherein R⁵ is H, F, Cl, Br, methyl, or methoxy.

24-26. (canceled)

27. The compound of claim 1, wherein R⁶ is H, F, or methoxy; and R⁷ is H or methyl; or R⁶ and R⁷, taken together with their intervening atoms, form benzene.

28-33. (canceled)

34. The compound of claim 1, wherein R⁸ and R⁹ are each independently H or F.

35-37. (canceled)

38. The compound of claim 1, of the following structural formula: 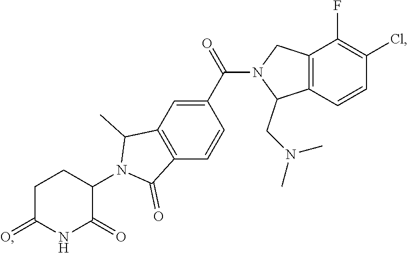 [SMILES: CC1c2cc(C(=O)N3Cc4c(ccc(Cl)c4F)C3CN(C)C)ccc2C(=O)N1C1CCC(=O)NC1=O]
or a pharmaceutically acceptable salt, tautomer, isotopologue, or stereoisomer thereof.

39. The compound of claim 38, of the following structural formula: 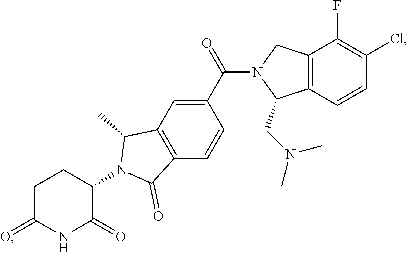 [SMILES: C[C@@H]1c2cc(C(=O)N3Cc4c(ccc(Cl)c4F)[C@@H]3CN(C)C)ccc2C(=O)N1[C@H]1CCC(=O)NC1=O]
or a pharmaceutically acceptable salt thereof.

40. The compound of claim 1, of the following structural formula: 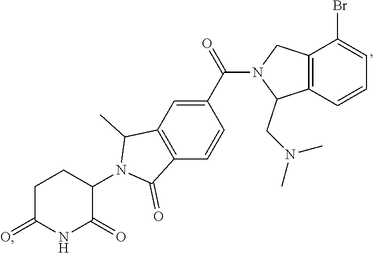 [SMILES: CC1c2cc(C(=O)N3Cc4c(Br)cccc4C3CN(C)C)ccc2C(=O)N1C1CCC(=O)NC1=O]
or a pharmaceutically acceptable salt, tautomer, isotopologue, or stereoisomer thereof.

41. The compound of claim 40, of the following structural formula: 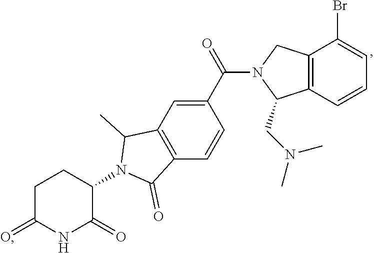 [SMILES: CC1c2cc(C(=O)N3Cc4c(Br)cccc4[C@@H]3CN(C)C)ccc2C(=O)N1[C@H]1CCC(=O)NC1=O]
or a pharmaceutically acceptable salt thereof.

42. The compound of claim 1, of the following structural formula: 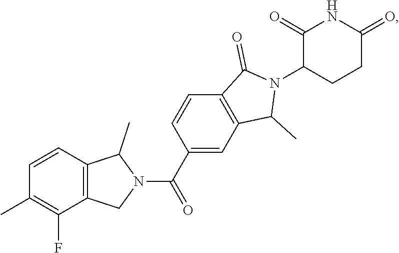 [SMILES: Cc1ccc2c(c1F)CN(C(=O)c1ccc3c(c1)C(C)N(C1CCC(=O)NC1=O)C3=O)C2C]
or a pharmaceutically acceptable salt, tautomer, isotopologue, or stereoisomer thereof.

43. The compound of claim 42, of the following structural formula: 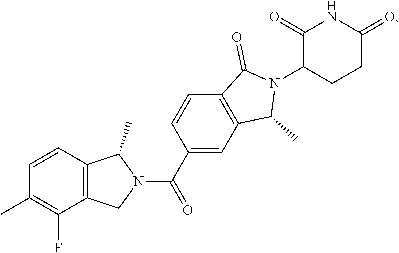 [SMILES: Cc1ccc2c(c1F)CN(C(=O)c1ccc3c(c1)[C@@H](C)N(C1CCC(=O)NC1=O)C3=O)[C@H]2C]
or a pharmaceutically acceptable salt thereof.

44. The compound of claim 1, of the following structural formula: 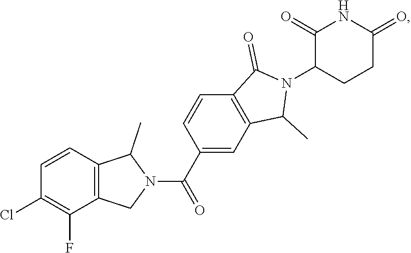 [SMILES: CC1c2ccc(Cl)c(F)c2CN1C(=O)c1ccc2c(c1)C(C)N(C1CCC(=O)NC1=O)C2=O]
or a pharmaceutically acceptable salt, tautomer, isotopologue, or stereoisomer thereof.

45. The compound of claim 44, of the following structural formula: 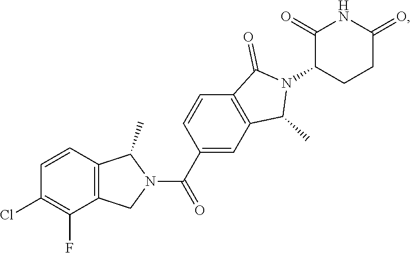 [SMILES: C[C@@H]1c2cc(C(=O)N3Cc4c(ccc(Cl)c4F)[C@@H]3C)ccc2C(=O)N1[C@H]1CCC(=O)NC1=O]
or a pharmaceutically acceptable salt thereof.

46. The compound of claim 1, of the following structural formula: 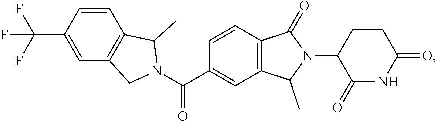 [SMILES: CC1c2ccc(C(F)(F)F)cc2CN1C(=O)c1ccc2c(c1)C(C)N(C1CCC(=O)NC1=O)C2=O]
or a pharmaceutically acceptable salt, tautomer, isotopologue, or stereoisomer thereof.

47. The compound of claim 46, of the following structural formula: 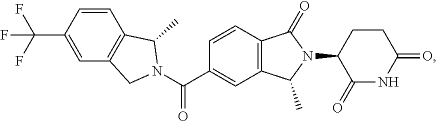 [SMILES: C[C@@H]1c2cc(C(=O)N3Cc4cc(C(F)(F)F)ccc4[C@@H]3C)ccc2C(=O)N1[C@H]1CCC(=O)NC1=O]
or a pharmaceutically acceptable salt thereof.

48. The compound of claim 1, of the following structural formula: 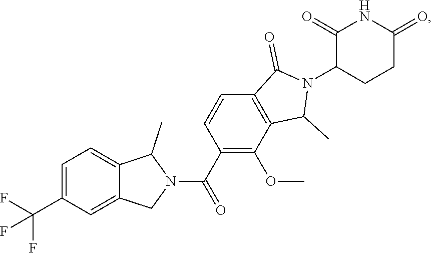 [SMILES: COc1c(C(=O)N2Cc3cc(C(F)(F)F)ccc3C2C)ccc2c1C(C)N(C1CCC(=O)NC1=O)C2=O]
or a pharmaceutically acceptable salt, tautomer, isotopologue, or stereoisomer thereof.

49. The compound of claim 48, of the following structural formula: 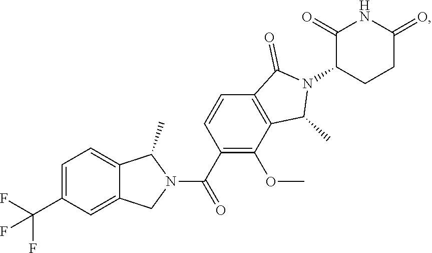 [SMILES: COc1c(C(=O)N2Cc3cc(C(F)(F)F)ccc3[C@@H]2C)ccc2c1[C@@H](C)N([C@H]1CCC(=O)NC1=O)C2=O]
or a pharmaceutically acceptable salt thereof.

50. The compound of claim 1, of the following structural formula: 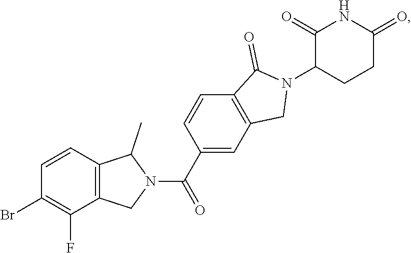 [SMILES: CC1c2ccc(Br)c(F)c2CN1C(=O)c1ccc2c(c1)CN(C1CCC(=O)NC1=O)C2=O]
or a pharmaceutically acceptable salt, tautomer, isotopologue, or stereoisomer thereof.

51. The compound of claim 50, of the following structural formula: 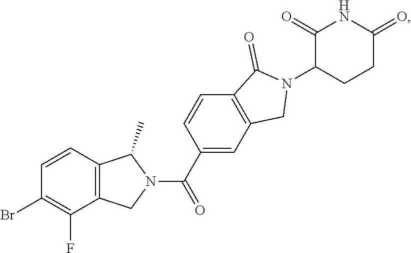 [SMILES: C[C@H]1c2ccc(Br)c(F)c2CN1C(=O)c1ccc2c(c1)CN(C1CCC(=O)NC1=O)C2=O]
or a pharmaceutically acceptable salt thereof.

52. The compound of claim 1, of the following structural formula: 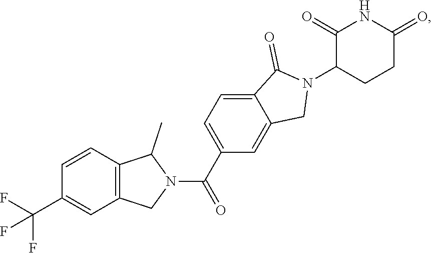 [SMILES: CC1c2ccc(C(F)(F)F)cc2CN1C(=O)c1ccc2c(c1)CN(C1CCC(=O)NC1=O)C2=O]
or a pharmaceutically acceptable salt, tautomer, isotopologue, or stereoisomer thereof.

53. The compound of claim 52, of the following structural formula: 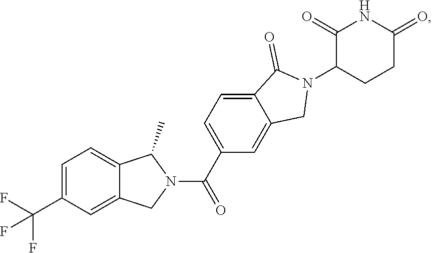 [SMILES: C[C@H]1c2ccc(C(F)(F)F)cc2CN1C(=O)c1ccc2c(c1)CN(C1CCC(=O)NC1=O)C2=O]
or a pharmaceutically acceptable salt thereof.

54. A pharmaceutical composition comprising a compound or pharmaceutically acceptable salt, tautomer, isotopologue, or stereoisomer thereof of claim 1 and a pharmaceutically acceptable excipient.

55. A method of modulating aryl hydrocarbon receptor nuclear translocator (ARNT) activity in a cell, comprising contacting the cell with a compound or pharmaceutically acceptable salt, tautomer, isotopologue, or stereoisomer thereof of claim 1.

56. A method of modulating aryl hydrocarbon receptor nuclear translocator (ARNT) activity in a subject in need thereof, comprising administering to the subject a therapeutically effective amount of a compound or pharmaceutically acceptable salt, tautomer, isotopologue, or stereoisomer thereof of claim 1.

57. A method of treating cancer in a subject in need thereof, comprising administering to the subject a therapeutically effective amount of a compound or pharmaceutically acceptable salt, tautomer, isotopologue, or stereoisomer thereof of claim 1.

58-61. (canceled)
